(19) United States
(12) Patent Application Publication
(10) Pub. No.: US 2026/0049118 A1
(43) Pub. Date: Feb. 19, 2026

(54) SINGLE-CHAIN CHIMERIC POLYPEPTIDES AND USES THEREOF

(71) Applicant: HCW Biologics, Inc., Miramar, FL (US)

(72) Inventor: Hing C. Wong, Miramar, FL (US)

(73) Assignee: HCW Biologics, Inc., Miramar, FL (US)

(21) Appl. No.: 19/304,242

(22) Filed: Aug. 19, 2025

Related U.S. Application Data
(60) Provisional application No. 63/684,684, filed on Aug. 19, 2024.
(60) Provisional application No. 63/744,786, filed on Jan. 13, 2025.

(51) Int. Cl. C07K 14/725; A61K 38/00; C07K 14/54; C07K 14/71; C12N 5/0783

(52) U.S. Cl. CPC C07K 14/7051; C07K 14/54; C07K 14/5418; C07K 14/5443; C07K 14/71; C12N 5/0636; C12N 5/0646; A61K 38/00; C07K 2319/00

(57) ABSTRACT

Provided herein are single-chain chimeric polypeptides that include: (i) a first target-binding domain; (ii) a soluble T cell receptor β-chain constant region (TRβC) domain; and (iii) a second target-binding domain. Also provided here are methods of making and using these single-chain chimeric polypeptides and nucleic acids encoding these single-chain chimeric polypeptides.

### CROSS-REFERENCE TO RELATED APPLICATIONS

This application claims priority to: U.S. Provisional Patent Application No. 63/684,684, filed Aug. 19, 2024; and U.S. Provisional Patent Application No. 63/744,786, filed Jan. 13, 2025, each of which is herein incorporated by reference in its entirety.

### SEQUENCE LISTING

This application contains a Sequence Listing that has been submitted electronically as an XML file named “47039-0039001_SL.XML.” The XML file, created on Aug. 19, 2025, is 156,118 bytes in size. The material in the XML file is hereby incorporated by reference in its entirety.

### TECHNICAL FIELD

The present disclosure relates to the field of biotechnology, and more specifically, to target-binding molecules.

### BACKGROUND

The T cell receptor (TCR) is expressed by the majority of mature T cell lymphomas (and ˜30% of T cell acute lymphoblastic leukemias). The TCR comprises a heterodimeric protein complex of two chains, TCRα and TCRβ. An ancestral duplication of the human β-chain constant gene results in the expression of one of two highly homologous chains, T cell receptor β-chain constant (TRβC) domains 1 and 2, in a mutually exclusive manner, following TCR locus rearrangement.

Human TRβC1 and TRβC2 isoforms differ by only four amino acid mutations in the extracellular domain. Otherwise, the sequence is identical between the two isoforms and the folded structure remains largely unchanged. While healthy T cells randomly express either TRβC1 or TRβC2, clonally expanded mature T cell lymphomas express one of them exclusively.

### SUMMARY

The present invention is based on the discovery that T-cell receptor β-chain constant region domains 1 or 2 (TRβC1 or TRβC2) can be used as a scaffold for chimeric polypeptides including a target-binding domain. Based on this discovery provided herein are single-chain chimeric polypeptides that include: (i) a first target-binding domain; (ii) a soluble T cell receptor β-chain constant region (TRβC) domain; and (iii) a second target-binding domain. Also provided herein are compositions that include any of the single-chain chimeric polypeptides described herein, nucleic acids that encode any of the single-chain chimeric polypeptides described herein, and cells that include any of the nucleic acids that encode any of the single-chain chimeric polypeptides described herein. Also provided herein are methods of stimulating an immune cell and methods of treating a subject in need thereof that include the use of any of the single-chain chimeric polypeptides described herein. Also provided herein are methods of producing any of the single-chain chimeric polypeptides described herein. Additional uses of any of the single-chain chimeric polypeptides are described herein.

Accordingly, provided herein is a single-chain chimeric polypeptides comprising: (i) a first target-binding domain; (ii) a soluble T cell receptor β-chain constant region (TRβC) domain; and (iii) a second target-binding domain.

In some embodiments, the first target-binding domain and the soluble TRβC domain directly abut each other. In some embodiments, the single-chain chimeric polypeptide further comprises a linker sequence between the first target-binding domain and the soluble TRβC domain. In some embodiments, the soluble TRβC domain and the second target-binding domain directly abut each other. In some embodiments, the single-chain chimeric polypeptide further comprises a linker sequence between the soluble TRβC domain and the second target-binding domain.

In some embodiments, the first target-binding domain and the second target-binding domain bind specifically to the same antigen. In some embodiments, the first target-binding domain and the second target-binding domain bind specifically to the same epitope. In some embodiments, the first target-binding domain and the second target-binding domain comprise the same amino acid sequence. In some embodiments, the first target-binding domain and the second target-binding domain bind specifically to different antigens. In some embodiments, one or both of the first target-binding domain and the second target-binding domain is an antigen-binding domain. In some embodiments, one or both of the first target-binding domain and the second target-binding domain bind to a target selected from the group consisting of: tissue factor (CD142), CD16a, CD28, CD3, CD33, CD20, CD19, CD22, CD123, IL-1R, IL-1, VEGF, IL-6R, IL-4, IL-10, PDL-1, TIGIT, PD-1, LCA-3, TIM3, CTLA4, MICA, MICB, IL-6, IL-8, TNFα, CD26, CD26a, CD36, ULBP2, CD30, CD40, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein, HLA-DR, DLL4, TYRO3, AXL, MER, CD122, CD155, PDGFDD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand for ICOS, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28. In some embodiments, one or both of the first target-binding domain and the second target-binding domain is a soluble interleukin, a soluble cytokine protein, or a soluble cell surface protein. In some embodiments, the soluble interleukin, soluble cytokine protein, or soluble cell surface protein is selected from the group consisting of: IL-1, IL-2, IL-3, IL-4, IL-7, IL-8, IL-10, IL-12, IL-15, IL-17, IL-18, IL-21, PDGF-D, SCF, FLT3L, MICA, MICB, and a ULP16-binding protein. In some embodiments, the soluble interleukin, soluble cytokine protein, or soluble cell surface protein is selected from the group consisting of: IL-7, IL-15, and IL-21. In some embodiments, one or both of the first target-binding domain and the second target-binding domain is a soluble interleukin receptor, a soluble cytokine receptor, or a soluble cell surface receptor. In some embodiments, the soluble interleukin receptor, the soluble cytokine receptor, or the soluble cell surface receptor is a soluble TGF-β receptor II (TGF-βRII), a soluble TGF-βRIII, a soluble NKG2D, a soluble NKp30, a soluble NKp44, a soluble NKp46, a soluble DNAM-1, a scMHCI, a scMHCII, a scTCR, a soluble CD155, or a soluble CD28. In some embodiments, the soluble interleukin receptor, the soluble cytokine receptor, or the soluble cell surface receptor is a soluble TGF-βRII.

In some embodiments, the first target-binding domain is IL-7 and the second target-binding domain is a soluble TGF-βRII. In some embodiments, the first target-binding domain is a soluble TGF-βRII and the second target-binding domain is a soluble TGF-βRII. In some embodiments, the first target-binding domain is IL-7 and the second target-binding domain is a soluble IL-21. In some embodiments, the first target-binding domain comprises a sequence that is at least 80% identical to SEQ ID NO: 4. In some embodiments, the second target-binding domain comprises a sequence that is at least 80% identical to SEQ ID NO: 5. In some embodiments, one or both of the first target-binding domain and the second target-binding domain comprises a first sequence that is at least 80% identical to SEQ ID NO: 7 and a second sequence that is at least 80% identical to SEQ ID NO: 7, wherein the first and second sequence are separated by a linker. In some embodiments, one or both of the first target-binding domain and the second target-binding domain comprises a sequence that is at least 80% identical to SEQ ID NO: 9 or 21. In some embodiments, one or both of the first target-binding domain and the second target-binding domain comprises a sequence that is at least 80% identical to wildtype IL-15.

In some embodiments, the single-chain chimeric polypeptide further comprises one or more additional target-binding domains at its N- and/or C-terminus.

In some embodiments, the soluble TRβC domain is a soluble human TRβC domain. In some embodiments, the soluble TRβC domain does not bind to an MHC protein with or without a loaded antigen. In some embodiments, the soluble TRβC domain does not stimulate an immune response in a mammal. In some embodiments, the soluble TRβC domain comprises or consists of a sequence from a wildtype soluble human TRβC. In some embodiments, the soluble TRβC domain comprises a sequence that is at least 80% identical to SEQ ID NO: 1. In some embodiments, the soluble human TRβC domain comprises a sequence that is at least 80% identical to SEQ ID NO: 3.

Also provided herein is a composition comprising any of the single-chain chimeric polypeptides discussed above. In some embodiments, the composition is a pharmaceutical composition. In some embodiments, a kit comprising at least one dose of any of the compositions discussed above is provided herein.

Also provided herein is a method of stimulating an immune cell, the method comprising: contacting an immune cell with an effective amount of any of the single-chain chimeric polypeptides discussed above or any of the compositions discussed above.

Also provided herein is a method of inducing or increasing proliferation of an immune cell, the method comprising: contacting an immune cell with an effective amount of any of the single-chain chimeric polypeptides discussed above or any of the compositions discussed above.

Also provided herein is a method of inducing differentiation of an immune cell into a memory or memory-like immune cell, the method comprising: contacting an immune cell with an effective amount of any of the single-chain chimeric polypeptides discussed above or any of the compositions discussed above. In some embodiments, the immune cell is contacted in vitro. In some embodiments, the immune cell is contacted in vivo. In some embodiments, the immune cell is selected from the group consisting of: an immature thymocyte, a peripheral blood lymphocyte, a naïve T cell, a pluripotent Th cell precursor, a lymphoid progenitor cell, a Treg cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a senescent CD8⁺ T cell, a mast cell, a macrophage, a neutrophil, a dendritic cell, a basophil, an eosinophil, and a natural killer cell. In some embodiments, the CD8⁺ T cell is a progenitor exhausted CD8⁺ T (Tpex) cell, an effector memory CD8⁺ T (Tem) cell, a central memory CD8⁺ T (Tcm) cell, a memory CD8⁺ stem (Tscm) cell, a virtual memory CD8⁺ T (Tvm) cell, an antigen-experienced CD8⁺ T cell, a terminally-exhausted CD8⁺ T (Tex) cell, or a senescent CD8⁺ cell. In some embodiments, the immune cell has previously been genetically modified to express a chimeric antigen receptor or a recombinant T-cell receptor. In some embodiments, the method further comprises administering the immune cell to a subject in need thereof.

Also provided herein is a method of killing a cancer cell, an infected cell, or a senescent cell in a subject in need thereof, the method comprising administering to the subject a therapeutically effective amount of any of the single-chain chimeric polypeptides discussed above or any of the compositions discussed above.

Also provided herein is a method of treating a subject in need thereof, the method comprising administering to the subject a therapeutically effective amount of any of the single-chain chimeric polypeptides discussed above or any of the compositions discussed above.

In some embodiments, the subject has been identified or diagnosed as having a cancer, an aging-related disease or condition, or an infectious disease. In some embodiments, the cancer is selected from the group consisting of: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma. In some embodiments, the aging-related disease or condition is selected from the group consisting of: a senescence-associated skin condition, atopic dermatitis, wrinkles, senile lentigo, age spots, photoaged skin, radiation-induced dermatitis, radiation-induced skin fibrosis/injury, cutaneous wound healing, Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, and renal dysfunction. In some embodiments, the infectious disease is infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus.

Also provided herein is a nucleic acid encoding any of the single-chain chimeric polypeptides discussed above. Some embodiments include a vector comprising the nucleic acid discussed above. In some embodiments, the vector is an expression vector. Some embodiments include a cell comprising the nucleic acid or the vector discussed above.

Also provided herein is a method of producing a single-chain chimeric polypeptide, the method comprising: culturing the cell in a culture medium under conditions sufficient to result in the production of the single-chain chimeric polypeptide; and recovering the single-chain chimeric polypeptide from the cell and/or the culture medium. Some embodiments comprise single-chain chimeric polypeptide produced by the discussed above. A “single-chain polypeptide” as used herein refers to a single protein chain.

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally-occurring human proteins. In some examples, a chimeric polypeptide can include a domain that is a synthetic sequence (e.g., a scFv) and a domain that is derived from a naturally-occurring protein (e.g., a naturally-occurring human protein). In some embodiments, a chimeric polypeptide can include at least two different domains that are synthetic sequences (e.g., two different scFvs).

A “T cell receptor β-chain constant region domain” refers to a polypeptide having at least 70% identity (e.g., at least 75% identity, at least 80% identity, at least 85% identity, at least 90% identity, at least 95% identity, at least 99% identity, or 100% identical) to a segment of a constant region of a wildtype mammalian T cell receptor β-chain (e.g., a wildtype human T cell receptor β-chain). Difference species have 1 to 5 different genes encoding the T cell receptor β-chain constant region domain. Human and mice have genes encoding the TRβC1 or TRβC2 isoforms. Non-limiting examples of T cell receptor β-chain constant region domains are described herein.

The term “soluble interleukin protein” is used herein to refer to a mature and secreted interleukin protein or a biologically active fragment thereof. In some examples, a soluble interleukin protein can include a sequence that is at least 70% identical, at least 75% identical, at least 80% identical, at least 85% identical, at least 90% identical, at least 95% identical, at least 99% identical, or 100% identical to a wildtype mature and secreted mammalian interleukin protein (e.g., a wildtype human interleukin protein) and retains its biological activity. Non-limiting examples of soluble interleukin proteins are described herein.

The term “soluble cytokine protein” is used herein to refer to a mature and secreted cytokine protein or a biologically active fragment thereof. In some examples, a soluble cytokine protein can include a sequence that is at least 70% identical, at least 75% identical, at least 80% identical, at least 85% identical, at least 90% identical, at least 95% identical, at least 99% identical, or 100% identical to a wildtype mature and secreted mammalian cytokine protein (e.g., a wildtype human cytokine protein) and retains its biological activity. Non-limiting examples of soluble cytokine proteins are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a V_{H}-V_{L} or a V_{L}-V_{H}. Another example of an antigen-binding domain is an antigen-binding domain formed by a V_{H}-V_{L} dimer or V_{L}-V_{H} dimer. Additional examples of an antibody are described herein. Additional examples of an antibody are known in the art.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D})). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO-OCTET®). Additional methods for determining the affinity for an antigen-binding domain and its corresponding antigen or epitope are known in the art.

The term “pair of affinity domains” is two different protein domain(s) that bind specifically to each other with a K_{D} of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

An “immune effector cell” refers to a cell of the immune system of a mammal that is capable, directly or indirectly, of recognizing and/or causing cytostasis or cell death of a pathogenic cell (e.g., a cancer cell) in the mammal. Non-limiting examples of immune effector cells include macrophages, T-lymphocytes (e.g., cytotoxic T-lymphocytes and T-helper cells), natural killer cells, neutrophils, monocytes, and eosinophils. Additional examples of immune effector cells are known in the art.

The term “treatment” means to ameliorate at least one symptom of a disorder. In some examples, the disorder being treated is cancer and to ameliorate at least one symptom of cancer includes reducing aberrant proliferation, gene expression, signaling, translation, and/or secretion of factors. Generally, the methods of treatment include administering a therapeutically effective amount of composition that reduces at least one symptom of a disorder to a subject who is in need of, or who has been determined to be in need of such treatment.

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human); or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine or primate animals) may be employed.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a schematic of an exemplary single-chain chimeric polypeptide: (i) a first target-binding domain; (ii) a soluble T cell receptor (TCR) β-chain constant region (TRβC) domain; and (iii) a second target-binding domain.

FIG. 2 is a schematic diagram of an exemplary single-chain chimeric polypeptide: (i) a first target-binding domain; (ii) a soluble T cell receptor (TCR) β-chain constant region (TRβC) domain; and (iii) a second target-binding domain.

FIG. 3 is a schematic diagram of an exemplary IL-7/TRβC1/TGFβRII single-chain chimeric polypeptide.

FIG. 4 is a schematic diagram of an exemplary IL-7/TRβC1/TGFβRII single-chain chimeric polypeptide.

FIG. 5 is a schematic diagram of an exemplary TGFβRII/hTRβC1/TGFβRII single-chain chimeric polypeptide.

FIG. 6 is a schematic diagram of an exemplary TGFβRII/hTRβC1/TGFβRII single-chain chimeric polypeptide.

FIG. 7 is a schematic diagram of an exemplary IL-7/hTRβC1/IL-21 single-chain chimeric polypeptide.

FIG. 8 is a schematic diagram of an exemplary IL-7/hTRβC1/IL-21 single-chain chimeric polypeptide.

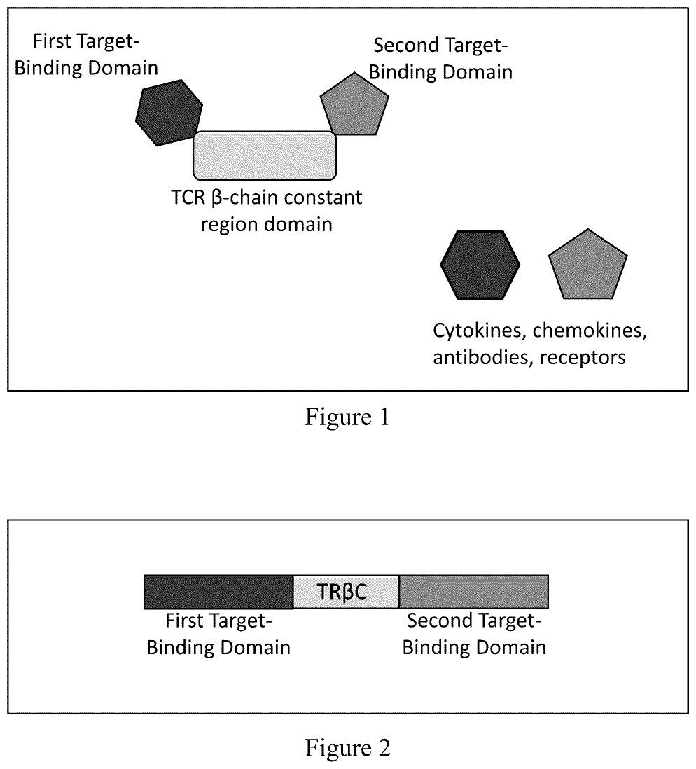

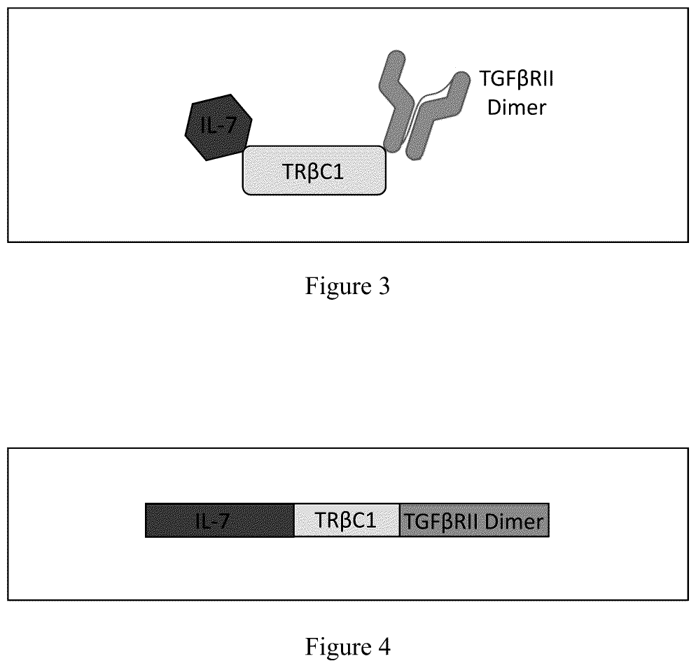

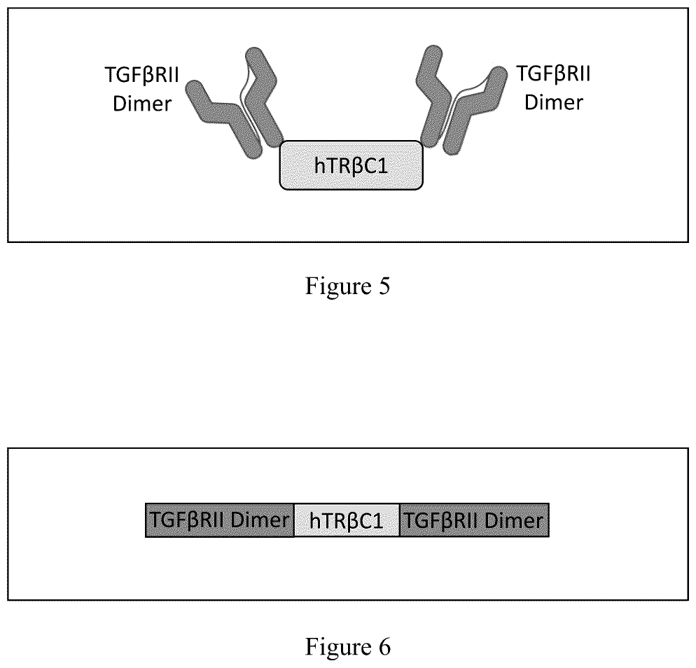

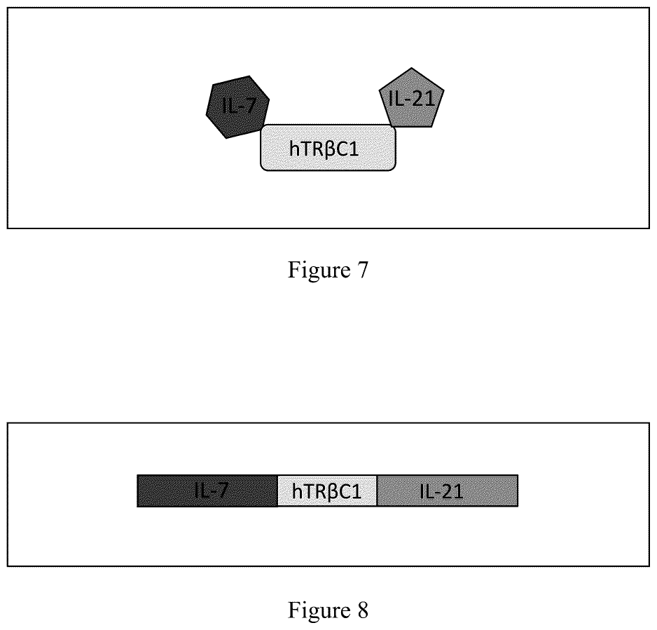

### DETAILED DESCRIPTION

Provided herein are single-chain chimeric polypeptides that include: (i) a first target-binding domain (e.g., any of the exemplary target-binding domains described herein or known in the art), (ii) a soluble T cell receptor β-chain constant region (TRβC) domain (e.g., any of the exemplary soluble TRβC domains described herein or known in the art), and (iii) a second target-binding domain (e.g., any of the exemplary target-binding domains described herein or known in the art).

Also provided herein are compositions that include any of the single-chain chimeric polypeptides described herein, nucleic acids that encode any of the single-chain chimeric polypeptides described herein, and cells that include any of the nucleic acids that encode any of the single-chain chimeric polypeptides described herein. Also provided herein are methods of stimulating an immune cell and methods of treating a subject in need thereof that include the use of any of the single-chain chimeric polypeptides described herein. Also provided herein are methods of producing any of the single-chain chimeric polypeptides described herein.

In some examples of any of the single-chain chimeric polypeptides described herein, the single-chain chimeric polypeptide can have a total length of about 50 amino acids to about 3000 amino acids, about 50 amino acids to about 2500 amino acids, about 50 amino acids to about 2000 amino acids, about 50 amino acids to about 1500 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 480 amino acids, about 50 amino acids to about 460 amino acids, about 50 amino acids to about 440 amino acids, about 50 amino acids to about 420 amino acids, about 50 amino acids to about 400 amino acids, about 50 amino acids to about 380 amino acids, about 50 amino acids to about 360 amino acids, about 50 amino acids to about 340 amino acids, about 50 amino acids to about 320 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 150 amino acids, about 50 amino acids to about 100 amino acids, about 100 amino acids to about 3000 amino acids, about 100 amino acids to about 2500 amino acids, about 100 amino acids to about 2000 amino acids, about 100 amino acids to about 1500 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 480 amino acids, about 100 amino acids to about 460 amino acids, about 100 amino acids to about 440 amino acids, about 100 amino acids to about 420 amino acids, about 100 amino acids to about 400 amino acids, about 100 amino acids to about 380 amino acids, about 100 amino acids to about 360 amino acids, about 100 amino acids to about 340 amino acids, about 100 amino acids to about 320 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 150 amino acids, about 150 amino acids to about 3000 amino acids, about 150 amino acids to about 2500 amino acids, about 150 amino acids to about 2000 amino acids, about 150 amino acids to about 1500 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 480 amino acids, about 150 amino acids to about 460 amino acids, about 150 amino acids to about 440 amino acids, about 150 amino acids to about 420 amino acids, about 150 amino acids to about 400 amino acids, about 150 amino acids to about 380 amino acids, about 150 amino acids to about 360 amino acids, about 150 amino acids to about 340 amino acids, about 150 amino acids to about 320 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 200 amino acids to about 3000 amino acids, about 200 amino acids to about 2500 amino acids, about 200 amino acids to about 2000 amino acids, about 200 amino acids to about 1500 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 480 amino acids, about 200 amino acids to about 460 amino acids, about 200 amino acids to about 440 amino acids, about 200 amino acids to about 420 amino acids, about 200 amino acids to about 400 amino acids, about 200 amino acids to about 380 amino acids, about 200 amino acids to about 360 amino acids, about 200 amino acids to about 340 amino acids, about 200 amino acids to about 320 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 3000 amino acids, about 220 amino acids to about 2500 amino acids, about 220 amino acids to about 2000 amino acids, about 220 amino acids to about 1500 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 480 amino acids, about 220 amino acids to about 460 amino acids, about 220 amino acids to about 440 amino acids, about 220 amino acids to about 420 amino acids, about 220 amino acids to about 400 amino acids, about 220 amino acids to about 380 amino acids, about 220 amino acids to about 360 amino acids, about 220 amino acids to about 340 amino acids, about 220 amino acids to about 320 amino acids, about 220 amino acids to about 300 amino acids, about 220 amino acids to about 280 amino acids, about 220 amino acids to about 260 amino acids, about 220 amino acids to about 240 amino acids, about 240 amino acids to about 3000 amino acids, about 240 amino acids to about 2500 amino acids, about 240 amino acids to about 2000 amino acids, about 240 amino acids to about 1500 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 480 amino acids, about 240 amino acids to about 460 amino acids, about 240 amino acids to about 440 amino acids, about 240 amino acids to about 420 amino acids, about 240 amino acids to about 400 amino acids, about 240 amino acids to about 380 amino acids, about 240 amino acids to about 360 amino acids, about 240 amino acids to about 340 amino acids, about 240 amino acids to about 320 amino acids, about 240 amino acids to about 300 amino acids, about 240 amino acids to about 280 amino acids, about 240 amino acids to about 260 amino acids, about 260 amino acids to about 3000 amino acids, about 260 amino acids to about 2500 amino acids, about 260 amino acids to about 2000 amino acids, about 260 amino acids to about 1500 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 480 amino acids, about 260 amino acids to about 460 amino acids, about 260 amino acids to about 440 amino acids, about 260 amino acids to about 420 amino acids, about 260 amino acids to about 400 amino acids, about 260 amino acids to about 380 amino acids, about 260 amino acids to about 360 amino acids, about 260 amino acids to about 340 amino acids, about 260 amino acids to about 320 amino acids, about 260 amino acids to about 300 amino acids, about 260 amino acids to about 280 amino acids, about 280 amino acids to about 3000 amino acids, about 280 amino acids to about 2500 amino acids, about 280 amino acids to about 2000 amino acids, about 280 amino acids to about 1500 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 480 amino acids, about 280 amino acids to about 460 amino acids, about 280 amino acids to about 440 amino acids, about 280 amino acids to about 420 amino acids, about 280 amino acids to about 400 amino acids, about 280 amino acids to about 380 amino acids, about 280 amino acids to about 360 amino acids, about 280 amino acids to about 340 amino acids, about 280 amino acids to about 320 amino acids, about 280 amino acids to about 300 amino acids, about 300 amino acids to about 3000 amino acids, about 300 amino acids to about 2500 amino acids, about 300 amino acids to about 2000 amino acids, about 300 amino acids to about 1500 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 480 amino acids, about 300 amino acids to about 460 amino acids, about 300 amino acids to about 440 amino acids, about 300 amino acids to about 420 amino acids, about 300 amino acids to about 400 amino acids, about 300 amino acids to about 380 amino acids, about 300 amino acids to about 360 amino acids, about 300 amino acids to about 340 amino acids, about 300 amino acids to about 320 amino acids, about 320 amino acids to about 3000 amino acids, about 320 amino acids to about 2500 amino acids, about 320 amino acids to about 2000 amino acids, about 320 amino acids to about 1500 amino acids, about 320 amino acids to about 1000 amino acids, about 320 amino acids to about 950 amino acids, about 320 amino acids to about 900 amino acids, about 320 amino acids to about 850 amino acids, about 320 amino acids to about 800 amino acids, about 320 amino acids to about 750 amino acids, about 320 amino acids to about 700 amino acids, about 320 amino acids to about 650 amino acids, about 320 amino acids to about 600 amino acids, about 320 amino acids to about 550 amino acids, about 320 amino acids to about 500 amino acids, about 320 amino acids to about 480 amino acids, about 320 amino acids to about 460 amino acids, about 320 amino acids to about 440 amino acids, about 320 amino acids to about 420 amino acids, about 320 amino acids to about 400 amino acids, about 320 amino acids to about 380 amino acids, about 320 amino acids to about 360 amino acids, about 320 amino acids to about 340 amino acids, about 340 amino acids to about 3000 amino acids, about 340 amino acids to about 2500 amino acids, about 340 amino acids to about 2000 amino acids, about 340 amino acids to about 1500 amino acids, about 340 amino acids to about 1000 amino acids, about 340 amino acids to about 950 amino acids, about 340 amino acids to about 900 amino acids, about 340 amino acids to about 850 amino acids, about 340 amino acids to about 800 amino acids, about 340 amino acids to about 750 amino acids, about 340 amino acids to about 700 amino acids, about 340 amino acids to about 650 amino acids, about 340 amino acids to about 600 amino acids, about 340 amino acids to about 550 amino acids, about 340 amino acids to about 500 amino acids, about 340 amino acids to about 480 amino acids, about 340 amino acids to about 460 amino acids, about 340 amino acids to about 440 amino acids, about 340 amino acids to about 420 amino acids, about 340 amino acids to about 400 amino acids, about 340 amino acids to about 380 amino acids, about 340 amino acids to about 360 amino acids, about 360 amino acids to about 3000 amino acids, about 360 amino acids to about 2500 amino acids, about 360 amino acids to about 2000 amino acids, about 360 amino acids to about 1500 amino acids, about 360 amino acids to about 1000 amino acids, about 360 amino acids to about 950 amino acids, about 360 amino acids to about 900 amino acids, about 360 amino acids to about 850 amino acids, about 360 amino acids to about 800 amino acids, about 360 amino acids to about 750 amino acids, about 360 amino acids to about 700 amino acids, about 360 amino acids to about 650 amino acids, about 360 amino acids to about 600 amino acids, about 360 amino acids to about 550 amino acids, about 360 amino acids to about 500 amino acids, about 360 amino acids to about 480 amino acids, about 360 amino acids to about 460 amino acids, about 360 amino acids to about 440 amino acids, about 360 amino acids to about 420 amino acids, about 360 amino acids to about 400 amino acids, about 360 amino acids to about 380 amino acids, about 380 amino acids to about 3000 amino acids, about 380 amino acids to about 2500 amino acids, about 380 amino acids to about 2000 amino acids, about 380 amino acids to about 1500 amino acids, about 380 amino acids to about 1000 amino acids, about 380 amino acids to about 950 amino acids, about 380 amino acids to about 900 amino acids, about 380 amino acids to about 850 amino acids, about 380 amino acids to about 800 amino acids, about 380 amino acids to about 750 amino acids, about 380 amino acids to about 700 amino acids, about 380 amino acids to about 650 amino acids, about 380 amino acids to about 600 amino acids, about 380 amino acids to about 550 amino acids, about 380 amino acids to about 500 amino acids, about 380 amino acids to about 480 amino acids, about 380 amino acids to about 460 amino acids, about 380 amino acids to about 440 amino acids, about 380 amino acids to about 420 amino acids, about 380 amino acids to about 400 amino acids, about 400 amino acids to about 3000 amino acids, about 400 amino acids to about 2500 amino acids, about 400 amino acids to about 2000 amino acids, about 400 amino acids to about 1500 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 480 amino acids, about 400 amino acids to about 460 amino acids, about 400 amino acids to about 440 amino acids, about 400 amino acids to about 420 amino acids, about 420 amino acids to about 3000 amino acids, about 420 amino acids to about 2500 amino acids, about 420 amino acids to about 2000 amino acids, about 420 amino acids to about 1500 amino acids, about 420 amino acids to about 1000 amino acids, about 420 amino acids to about 950 amino acids, about 420 amino acids to about 900 amino acids, about 420 amino acids to about 850 amino acids, about 420 amino acids to about 800 amino acids, about 420 amino acids to about 750 amino acids, about 420 amino acids to about 700 amino acids, about 420 amino acids to about 650 amino acids, about 420 amino acids to about 600 amino acids, about 420 amino acids to about 550 amino acids, about 420 amino acids to about 500 amino acids, about 420 amino acids to about 480 amino acids, about 420 amino acids to about 460 amino acids, about 420 amino acids to about 440 amino acids, about 440 amino acids to about 3000 amino acids, about 440 amino acids to about 2500 amino acids, about 440 amino acids to about 2000 amino acids, about 440 amino acids to about 1500 amino acids, about 440 amino acids to about 1000 amino acids, about 440 amino acids to about 950 amino acids, about 440 amino acids to about 900 amino acids, about 440 amino acids to about 850 amino acids, about 440 amino acids to about 800 amino acids, about 440 amino acids to about 750 amino acids, about 440 amino acids to about 700 amino acids, about 440 amino acids to about 650 amino acids, about 440 amino acids to about 600 amino acids, about 440 amino acids to about 550 amino acids, about 440 amino acids to about 500 amino acids, about 440 amino acids to about 480 amino acids, about 440 amino acids to about 460 amino acids, about 460 amino acids to about 3000 amino acids, about 460 amino acids to about 2500 amino acids, about 460 amino acids to about 2000 amino acids, about 460 amino acids to about 1500 amino acids, about 460 amino acids to about 1000 amino acids, about 460 amino acids to about 950 amino acids, about 460 amino acids to about 900 amino acids, about 460 amino acids to about 850 amino acids, about 460 amino acids to about 800 amino acids, about 460 amino acids to about 750 amino acids, about 460 amino acids to about 700 amino acids, about 460 amino acids to about 650 amino acids, about 460 amino acids to about 600 amino acids, about 460 amino acids to about 550 amino acids, about 460 amino acids to about 500 amino acids, about 460 amino acids to about 480 amino acids, about 480 amino acids to about 3000 amino acids, about 480 amino acids to about 2500 amino acids, about 480 amino acids to about 2000 amino acids, about 480 amino acids to about 1500 amino acids, about 480 amino acids to about 1000 amino acids, about 480 amino acids to about 950 amino acids, about 480 amino acids to about 900 amino acids, about 480 amino acids to about 850 amino acids, about 480 amino acids to about 800 amino acids, about 480 amino acids to about 750 amino acids, about 480 amino acids to about 700 amino acids, about 480 amino acids to about 650 amino acids, about 480 amino acids to about 600 amino acids, about 480 amino acids to about 550 amino acids, about 480 amino acids to about 500 amino acids, about 500 amino acids to about 3000 amino acids, about 500 amino acids to about 2500 amino acids, about 500 amino acids to about 2000 amino acids, about 500 amino acids to about 1500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 3000 amino acids, about 550 amino acids to about 2500 amino acids, about 550 amino acids to about 2000 amino acids, about 550 amino acids to about 1500 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 3000 amino acids, about 600 amino acids to about 2500 amino acids, about 600 amino acids to about 2000 amino acids, about 600 amino acids to about 1500 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 3000 amino acids, about 650 amino acids to about 2500 amino acids, about 650 amino acids to about 2000 amino acids, about 650 amino acids to about 1500 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 3000 amino acids, about 700 amino acids to about 2500 amino acids, about 700 amino acids to about 2000 amino acids, about 700 amino acids to about 1500 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 3000 amino acids, about 750 amino acids to about 2500 amino acids, about 750 amino acids to about 2000 amino acids, about 750 amino acids to about 1500 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 3000 amino acids, about 800 amino acids to about 2500 amino acids, about 800 amino acids to about 2000 amino acids, about 800 amino acids to about 1500 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 3000 amino acids, about 850 amino acids to about 2500 amino acids, about 850 amino acids to about 2000 amino acids, about 850 amino acids to about 1500 amino acids, about 850 amino acids to about 1000 amino acids, about 850 amino acids to about 950 amino acids, about 850 amino acids to about 900 amino acids, about 900 amino acids to about 3000 amino acids, about 900 amino acids to about 2500 amino acids, about 900 amino acids to about 2000 amino acids, about 900 amino acids to about 1500 amino acids, about 900 amino acids to about 1000 amino acids, about 900 amino acids to about 950 amino acids, about 950 amino acids to about 3000 amino acids, about 950 amino acids to about 2500 amino acids, about 950 amino acids to about 2000 amino acids, about 950 amino acids to about 1500 amino acids, about 950 amino acids to about 1000 amino acids, about 1000 amino acids to about 3000 amino acids, about 1000 amino acids to about 2500 amino acids, about 1000 amino acids to about 2000 amino acids, about 1000 amino acids to about 1500 amino acids, about 1500 amino acids to about 3000 amino acids, about 1500 amino acids to about 2500 amino acids, about 1500 amino acids to about 2000 amino acids, about 2000 amino acids to about 3000 amino acids, about 2000 amino acids to about 2500 amino acids, or about 2500 amino acids to about 3000 amino acids. Diagrams of an exemplary single-chain chimeric polypeptide provided herein are depicted in FIG. 1.

In some embodiments of any of the single-chain chimeric polypeptides described herein, the first target-binding domain (e.g., any of the exemplary target-binding domains described herein or known in the art) and the soluble TRβC domain (e.g., any of the exemplary soluble TRβC domains described herein) directly abut each other. In some embodiments of any of the single-chain chimeric polypeptides described herein, the single-chain chimeric polypeptide further comprises a linker sequence (e.g., any of the exemplary linker sequences described herein or known in the art) between the first target-binding domain (e.g., any of the exemplary target-binding domains described herein or known in the art) and the soluble TRβC domain (e.g., any of the exemplary soluble TRβC domains described herein). In some embodiments of any of the single-chain chimeric polypeptides described herein, the soluble TRβC domain (e.g., any of the exemplary soluble TRβC domains described herein) and the second target-binding domain (e.g., any of the exemplary target-binding domains described herein or known in the art) directly abut each other. In some embodiments of any of the single-chain chimeric polypeptides described herein, the single-chain chimeric polypeptide further comprises a linker sequence (e.g., any of the exemplary linker sequences described herein or known in the art) between the soluble TRβC domain (e.g., any of the exemplary soluble TRβC domains described herein) and the second target-binding domain (e.g., any of the exemplary target-binding domains described herein or known in the art).

In some embodiments of any of the single-chain chimeric polypeptides described herein, the first target-binding domain (e.g., any of the exemplary target-binding domains described herein or known in the art) and the second target-binding domain (e.g., any of the exemplary target-binding domains described herein or known in the art) directly abut each other. In some embodiments of any of the single-chain chimeric polypeptides described herein, the single-chain chimeric polypeptide further comprises a linker sequence (e.g., any of the exemplary linker sequences described herein or known in the art) between the first target-binding domain (e.g., any of the exemplary target-binding domains described herein or known in the art) and the second target-binding domain (e.g., any of the exemplary target-binding domains described herein or known in the art). In some embodiments of any of the single-chain chimeric polypeptides described herein, the second target-binding domain (e.g., any of the exemplary target-binding domains described herein or known in the art) and the soluble TRβC domain (e.g., any of the exemplary soluble TRβC domains described herein) directly abut each other. In some embodiments of any of the single-chain chimeric polypeptides described herein, the single-chain chimeric polypeptide further comprises a linker sequence (e.g., any of the exemplary linker sequences described herein or known in the art) between the second target-binding domain (e.g., any of the exemplary target-binding domains described herein or known in the art) and the soluble TRβC domain (e.g., any of the exemplary soluble TRβC domains described herein or known in the art).

Non-limiting aspects of these chimeric polypeptides, nucleic acids, vectors, cells, and methods are described below, and can be used in any combination without limitation. Additional aspects of these chimeric polypeptides, nucleic acids, vectors, cells, and methods are known in the art.

### T Cell Receptor β-Chain Constant Region (TRβC)

The T-cell receptor (TCR) is a heterodimer of two chains, an α-chain and a β-chain or a γ-chain and a δ-chain. An individual T cell can express either an αβ heterodimer or a γδ heterodimer as its receptor. The TCR is expressed with cluster of differentiation 3 (CD3) polypeptides and forms a complex comprising the TCR and six CD3 polypeptides (the TCR-CD3 complex). Three dimers of CD3 proteins, δε and γε heterodimers and ζζ homodimers, are present in the TCR-CD3 complex. TCR-CD3 complexes can be found on the surface of T cells where they recognize self or foreign antigens presented by major histocompatibility complex (MHC) molecules and can be involved in immune synapse formation, inducing intracellular signaling, and initiating target cell killing. The TCR is involved in antigen recognition, and the CD3 polypeptides contain intracellular signaling domains that couple the TCR-CD3 complex to the downstream signaling machinery.

TCR chains include an extracellular region, a transmembrane region, and a shorter cytoplasmic tail. The extracellular regions of TCR chains contain a variable (V) region and a constant (C) region. TCR diversity can be generated by somatic V(D)J recombination in which variable (V) regions and joining (J) regions or variable (V) regions, joining (J) regions, and diversity (D) regions can be combined during the production of a TCR chain (e.g., V, D and J for β and δ chains; V and J for α and γ chains). Cells in a clonal T cell population express the same unique TCR.

Difference species have 1 to 5 different genes encoding the T cell receptor β-chain constant region domain. Human and mice have genes encoding the TRβC1 (TRβC1 gene) or TRβC2 (TRβC2 gene) isoforms. Human TRβC1 and TRβC2 differ by only four amino acid mutations in the extracellular domain. The surrounding sequence is identical between the two isoforms and the folded structure remains largely unchanged. Each TCR (and therefore each T cell) selects a TCR β-chain constant region encoded by either TRβC1 or TRβC2 for expression in a mutually exclusive manner. Hence, a population of normal T cells will comprise a mixture of individual cells, some expressing TRβC1 and others expressing TRβC2, whereas the entire population of a T cell cancer will express either TRβC1 or TRβC2.

### Soluble T Cell Receptor β-Chain Constant Region (TRβC) Domains

In some embodiments of any of the single-chain chimeric polypeptides, compositions, or methods described herein, the soluble TRβC domain can be a soluble human TRβC domain. In some embodiments, the soluble TRβC domain can be a soluble human TRβC1 domain. In some embodiments, the soluble TRβC domain can be a soluble human TRβC2 domain. Non-limiting examples of soluble TRβC domains are shown below.

In some embodiments, a soluble T cell receptor β-chain constant region (TRβC) domain can comprise or consist of a sequence that is at least 80%, 85%, 90%, 92%, 95%, 96%, 98%, or 100% identical to SEQ ID NO: 1 or 3. In some embodiments, a soluble T cell receptor β-chain constant region (TRβC) domain is encoded by a nucleic acid that is at least 80%, 85%, 90%, 92%, 95%, 96%, 98%, or 100% identical to SEQ ID NO: 2.

Exemplary Soluble Human TRβC1 Domain
(SEQ ID NO: 1)
EDLNKVFPPEVAVFEPSEAEISHTQKATLVCLATGFFPDHVELSWWV
NGKEVHSGVSTDPQPLKEQPALNDSRYCLSSRLRVSATFWQNPRNHF
RAKPVTQIVSAEAWGRAD
Exemplary Nucleic Acid Encoding Soluble Human
TRβC1 Domain
(SEQ ID NO: 2)
GAGGACCTGAACAAGGTGTTCCCACCCGAGGTCGCTGTGTTTGAGCC
ATCAGAAGCAGAGATCTCCCACACCCAAAAGGCCACACTGGTGTGCC
TGGCCACAGGCTTCTTCCCCGACCACGTGGAGCTGAGCTGGTGGGTG
AATGGGAAGGAGGTGCACAGTGGGGTCAGCACGGACCCGCAGCCCCT
CAAGGAGCAGCCCGCCCTCAATGACTCCAGATACTGCCTGAGCAGCC
GCCTGAGGGTCTCGGCCACCTTCTGGCAGAACCCCCGCAACCACTTC
CGCTGTCAAGTCCAGTTCTACGGGCTCTCGGAGAATGACGAGTGGAC
CCAGGATAGGGCCAAACCCGTCACCCAGATCGTCAGCGCCGAGGCCT
GGGGTAGAGCAGAC
Exemplary Soluble Human TRβC2 Domain
(SEQ ID NO: 3)
EDLKNVFPPEVAVFEPSEAEISHTQKATLVCLATGFYPDHVELSWWV
NGKEVHSGVSTDPQPLKEQPALNDSRYCLSSRLRVSATFWQNPRNHF
RCQVQFYGLSENDEWTQDRAKPVTQIVSAEAWGRAD

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some embodiments, the soluble TRβC domain can comprise or consist of a soluble wildtype human TRβC2 domain (or any sequence therefrom).

In some embodiments, the soluble TRβC domain does not bind to an MHC protein with or without a loaded antigen. In some embodiments, the soluble TRβC domain does not stimulate an immune response in a mammal.

In some embodiments, the soluble TRβC domain can have a total length of about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 60 amino acids, about 30 amino acids to about 50 amino acids, about 30 amino acids to about 40 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 60 amino acids, about 40 amino acids to about 50 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 60 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, acids, about 135 amino acids to about 155 amino acids, about 135 amino acids to about 150 amino acids, about 135 amino acids to about 145 amino acids, about 135 amino acids to about 140 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, acids, about 140 amino acids to about 155 amino acids, about 140 amino acids to about 150 amino acids, about 140 amino acids to about 145 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, acids, about 145 amino acids to about 155 amino acids, about 145 amino acids to about 150 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, acids, about 150 amino acids to about 155 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 200 amino acids, about 180 amino acids to about 195 amino acids, about 180 amino acids to about 190 amino acids, about 180 amino acids to about 185 amino acids, about 185 amino acids to about 200 amino acids, about 185 amino acids to about 195 amino acids, about 185 amino acids to about 190 amino acids, about 190 amino acids to about 200 amino acids, about 190 amino acids to about 195 amino acids, or about 195 amino acids to about 200 amino acids.

### Target-Binding Domains

In some embodiments of any of the single-chain chimeric polypeptides described herein, the first target-binding domain, the second target-binding domain, and/or the additional one or more target-binding domains can be an antigen-binding domain (e.g., any of the exemplary antigen-binding domains described herein or known in the art), a soluble interleukin or cytokine protein (e.g., any of the exemplary soluble interleukin proteins or soluble cytokine proteins described herein), and a soluble interleukin or cytokine receptor (e.g., any of the exemplary soluble interleukin receptors or soluble cytokine receptors described herein).

In some embodiments of any of the single-chain chimeric polypeptides described herein, one or more of the first target-binding domain (e.g., any of the exemplary first target-binding domains described herein or known in the art), the second target-binding domain (e.g., any of the exemplary second target-binding domains described herein or known in the art), and the one or more additional target-binding domains can each, independently, bind specifically to a target selected from the group of: bind specifically to a target selected from the group consisting of: tissue factor (CD142), CD16a, CD28, CD3 (e.g., one or more of CD3α, CD3β, CD3δ, CD3ε, and CD3γ), CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein (e.g., ULBP1, ULBP2, ULBP3, ULBP4, ULBP5, and ULBP6), HLA-DR, DLL4, TYRO3, AXL, MER, CD122, CD155, PDGF-DD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand for ICOS, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of any of the single-chain chimeric polypeptides described herein, the first target-binding domain, the second target-binding domain, and/or the one or more additional target-binding domains can each independently have a total number of amino acids of about 5 amino acids to about 1000 amino acids, about 5 amino acids to about 950 amino acids, about 5 amino acids to about 900 amino acids, about 5 amino acids to about 850 amino acids, about 5 amino acids to about 800 amino acids, about 5 amino acids to about 750 amino acids, about 5 amino acids to about 700 amino acids, about 5 amino acids to about 650 amino acids, about 5 amino acids to about 600 amino acids, about 5 amino acids to about 550 amino acids, about 5 amino acids to about 500 amino acids, about 5 amino acids to about 450 amino acids, about 5 amino acids to about 400 amino acids, about 5 amino acids to about 350 amino acids, about 5 amino acids to about 300 amino acids, about 5 amino acids to about 280 amino acids, about 5 amino acids to about 260 amino acids, about 5 amino acids to about 240 amino acids, about 5 amino acids to about 220 amino acids, about 5 amino acids to about 200 amino acids, about 5 amino acids to about 195 amino acids, about 5 amino acids to about 190 amino acids, about 5 amino acids to about 185 amino acids, about 5 amino acids to about 180 amino acids, about 5 amino acids to about 175 amino acids, about 5 amino acids to about 170 amino acids, about 5 amino acids to about 165 amino acids, about 5 amino acids to about 160 amino acids, about 5 amino acids to about 155 amino acids, about 5 amino acids to about 150 amino acids, about 5 amino acids to about 145 amino acids, about 5 amino acids to about 140 amino acids, about 5 amino acids to about 135 amino acids, about 5 amino acids to about 130 amino acids, about 5 amino acids to about 125 amino acids, about 5 amino acids to about 120 amino acids, about 5 amino acids to about 115 amino acids, about 5 amino acids to about 110 amino acids, about 5 amino acids to about 105 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 1000 amino acids, about 10 amino acids to about 950 amino acids, about 10 amino acids to about 900 amino acids, about 10 amino acids to about 850 amino acids, about 10 amino acids to about 800 amino acids, about 10 amino acids to about 750 amino acids, about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 280 amino acids, about 10 amino acids to about 260 amino acids, about 10 amino acids to about 240 amino acids, about 10 amino acids to about 220 amino acids, about 10 amino acids to about 200 amino acids, about 10 amino acids to about 195 amino acids, about 10 amino acids to about 190 amino acids, about 10 amino acids to about 185 amino acids, about 10 amino acids to about 180 amino acids, about 10 amino acids to about 175 amino acids, about 10 amino acids to about 170 amino acids, about 10 amino acids to about 165 amino acids, about 10 amino acids to about 160 amino acids, about 10 amino acids to about 155 amino acids, about 10 amino acids to about 150 amino acids, about 10 amino acids to about 145 amino acids, about 10 amino acids to about 140 amino acids, about 10 amino acids to about 135 amino acids, about 10 amino acids to about 130 amino acids, about 10 amino acids to about 125 amino acids, about 10 amino acids to about 120 amino acids, about 10 amino acids to about 115 amino acids, about 10 amino acids to about 110 amino acids, about 10 amino acids to about 105 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 1000 amino acids, about 15 amino acids to about 950 amino acids, about 15 amino acids to about 900 amino acids, about 15 amino acids to about 850 amino acids, about 15 amino acids to about 800 amino acids, about 15 amino acids to about 750 amino acids, about 15 amino acids to about 700 amino acids, about 15 amino acids to about 650 amino acids, about 15 amino acids to about 600 amino acids, about 15 amino acids to about 550 amino acids, about 15 amino acids to about 500 amino acids, about 15 amino acids to about 450 amino acids, about 15 amino acids to about 400 amino acids, about 15 amino acids to about 350 amino acids, about 15 amino acids to about 300 amino acids, about 15 amino acids to about 280 amino acids, about 15 amino acids to about 260 amino acids, about 15 amino acids to about 240 amino acids, about 15 amino acids to about 220 amino acids, about 15 amino acids to about 200 amino acids, about 15 amino acids to about 195 amino acids, about 15 amino acids to about 190 amino acids, about 15 amino acids to about 185 amino acids, about 15 amino acids to about 180 amino acids, about 15 amino acids to about 175 amino acids, about 15 amino acids to about 170 amino acids, about 15 amino acids to about 165 amino acids, about 15 amino acids to about 160 amino acids, about 15 amino acids to about 155 amino acids, about 15 amino acids to about 150 amino acids, about 15 amino acids to about 145 amino acids, about 15 amino acids to about 140 amino acids, about 15 amino acids to about 135 amino acids, about 15 amino acids to about 130 amino acids, about 15 amino acids to about 125 amino acids, about 15 amino acids to about 120 amino acids, about 15 amino acids to about 115 amino acids, about 15 amino acids to about 110 amino acids, about 15 amino acids to about 105 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 280 amino acids, about 20 amino acids to about 260 amino acids, about 20 amino acids to about 240 amino acids, about 20 amino acids to about 220 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 280 amino acids, about 25 amino acids to about 260 amino acids, about 25 amino acids to about 240 amino acids, about 25 amino acids to about 220 amino acids, about 25 amino acids to about 200 amino acids, about 25 amino acids to about 195 amino acids, about 25 amino acids to about 190 amino acids, about 25 amino acids to about 185 amino acids, about 25 amino acids to about 180 amino acids, about 25 amino acids to about 175 amino acids, about 25 amino acids to about 170 amino acids, about 25 amino acids to about 165 amino acids, about 25 amino acids to about 160 amino acids, about 25 amino acids to about 155 amino acids, about 25 amino acids to about 150 amino acids, about 25 amino acids to about 145 amino acids, about 25 amino acids to about 140 amino acids, about 25 amino acids to about 135 amino acids, about 25 amino acids to about 130 amino acids, about 25 amino acids to about 125 amino acids, about 25 amino acids to about 120 amino acids, about 25 amino acids to about 115 amino acids, about 25 amino acids to about 110 amino acids, about 25 amino acids to about 105 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 280 amino acids, about 30 amino acids to about 260 amino acids, about 30 amino acids to about 240 amino acids, about 30 amino acids to about 220 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 280 amino acids, about 35 amino acids to about 260 amino acids, about 35 amino acids to about 240 amino acids, about 35 amino acids to about 220 amino acids, about 35 amino acids to about 200 amino acids, about 35 amino acids to about 195 amino acids, about 35 amino acids to about 190 amino acids, about 35 amino acids to about 185 amino acids, about 35 amino acids to about 180 amino acids, about 35 amino acids to about 175 amino acids, about 35 amino acids to about 170 amino acids, about 35 amino acids to about 165 amino acids, about 35 amino acids to about 160 amino acids, about 35 amino acids to about 155 amino acids, about 35 amino acids to about 150 amino acids, about 35 amino acids to about 145 amino acids, about 35 amino acids to about 140 amino acids, about 35 amino acids to about 135 amino acids, about 35 amino acids to about 130 amino acids, about 35 amino acids to about 125 amino acids, about 35 amino acids to about 120 amino acids, about 35 amino acids to about 115 amino acids, about 35 amino acids to about 110 amino acids, about 35 amino acids to about 105 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 280 amino acids, about 40 amino acids to about 260 amino acids, about 40 amino acids to about 240 amino acids, about 40 amino acids to about 220 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 280 amino acids, about 45 amino acids to about 260 amino acids, about 45 amino acids to about 240 amino acids, about 45 amino acids to about 220 amino acids, about 45 amino acids to about 200 amino acids, about 45 amino acids to about 195 amino acids, about 45 amino acids to about 190 amino acids, about 45 amino acids to about 185 amino acids, about 45 amino acids to about 180 amino acids, about 45 amino acids to about 175 amino acids, about 45 amino acids to about 170 amino acids, about 45 amino acids to about 165 amino acids, about 45 amino acids to about 160 amino acids, about 45 amino acids to about 155 amino acids, about 45 amino acids to about 150 amino acids, about 45 amino acids to about 145 amino acids, about 45 amino acids to about 140 amino acids, about 45 amino acids to about 135 amino acids, about 45 amino acids to about 130 amino acids, about 45 amino acids to about 125 amino acids, about 45 amino acids to about 120 amino acids, about 45 amino acids to about 115 amino acids, about 45 amino acids to about 110 amino acids, about 45 amino acids to about 105 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 1000 amino acids, about 55 amino acids to about 950 amino acids, about 55 amino acids to about 900 amino acids, about 55 amino acids to about 850 amino acids, about 55 amino acids to about 800 amino acids, about 55 amino acids to about 750 amino acids, about 55 amino acids to about 700 amino acids, about 55 amino acids to about 650 amino acids, about 55 amino acids to about 600 amino acids, about 55 amino acids to about 550 amino acids, about 55 amino acids to about 500 amino acids, about 55 amino acids to about 450 amino acids, about 55 amino acids to about 400 amino acids, about 55 amino acids to about 350 amino acids, about 55 amino acids to about 300 amino acids, about 55 amino acids to about 280 amino acids, about 55 amino acids to about 260 amino acids, about 55 amino acids to about 240 amino acids, about 55 amino acids to about 220 amino acids, about 55 amino acids to about 200 amino acids, about 55 amino acids to about 195 amino acids, about 55 amino acids to about 190 amino acids, about 55 amino acids to about 185 amino acids, about 55 amino acids to about 180 amino acids, about 55 amino acids to about 175 amino acids, about 55 amino acids to about 170 amino acids, about 55 amino acids to about 165 amino acids, about 55 amino acids to about 160 amino acids, about 55 amino acids to about 155 amino acids, about 55 amino acids to about 150 amino acids, about 55 amino acids to about 145 amino acids, about 55 amino acids to about 140 amino acids, about 55 amino acids to about 135 amino acids, about 55 amino acids to about 130 amino acids, about 55 amino acids to about 125 amino acids, about 55 amino acids to about 120 amino acids, about 55 amino acids to about 115 amino acids, about 55 amino acids to about 110 amino acids, about 55 amino acids to about 105 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 280 amino acids, about 60 amino acids to about 260 amino acids, about 60 amino acids to about 240 amino acids, about 60 amino acids to about 220 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 1000 amino acids, about 65 amino acids to about 950 amino acids, about 65 amino acids to about 900 amino acids, about 65 amino acids to about 850 amino acids, about 65 amino acids to about 800 amino acids, about 65 amino acids to about 750 amino acids, about 65 amino acids to about 700 amino acids, about 65 amino acids to about 650 amino acids, about 65 amino acids to about 600 amino acids, about 65 amino acids to about 550 amino acids, about 65 amino acids to about 500 amino acids, about 65 amino acids to about 450 amino acids, about 65 amino acids to about 400 amino acids, about 65 amino acids to about 350 amino acids, about 65 amino acids to about 300 amino acids, about 65 amino acids to about 280 amino acids, about 65 amino acids to about 260 amino acids, about 65 amino acids to about 240 amino acids, about 65 amino acids to about 220 amino acids, about 65 amino acids to about 200 amino acids, about 65 amino acids to about 195 amino acids, about 65 amino acids to about 190 amino acids, about 65 amino acids to about 185 amino acids, about 65 amino acids to about 180 amino acids, about 65 amino acids to about 175 amino acids, about 65 amino acids to about 170 amino acids, about 65 amino acids to about 165 amino acids, about 65 amino acids to about 160 amino acids, about 65 amino acids to about 155 amino acids, about 65 amino acids to about 150 amino acids, about 65 amino acids to about 145 amino acids, about 65 amino acids to about 140 amino acids, about 65 amino acids to about 135 amino acids, about 65 amino acids to about 130 amino acids, about 65 amino acids to about 125 amino acids, about 65 amino acids to about 120 amino acids, about 65 amino acids to about 115 amino acids, about 65 amino acids to about 110 amino acids, about 65 amino acids to about 105 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 280 amino acids, about 70 amino acids to about 260 amino acids, about 70 amino acids to about 240 amino acids, about 70 amino acids to about 220 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 1000 amino acids, about 75 amino acids to about 950 amino acids, about 75 amino acids to about 900 amino acids, about 75 amino acids to about 850 amino acids, about 75 amino acids to about 800 amino acids, about 75 amino acids to about 750 amino acids, about 75 amino acids to about 700 amino acids, about 75 amino acids to about 650 amino acids, about 75 amino acids to about 600 amino acids, about 75 amino acids to about 550 amino acids, about 75 amino acids to about 500 amino acids, about 75 amino acids to about 450 amino acids, about 75 amino acids to about 400 amino acids, about 75 amino acids to about 350 amino acids, about 75 amino acids to about 300 amino acids, about 75 amino acids to about 280 amino acids, about 75 amino acids to about 260 amino acids, about 75 amino acids to about 240 amino acids, about 75 amino acids to about 220 amino acids, about 75 amino acids to about 200 amino acids, about 75 amino acids to about 195 amino acids, about 75 amino acids to about 190 amino acids, about 75 amino acids to about 185 amino acids, about 75 amino acids to about 180 amino acids, about 75 amino acids to about 175 amino acids, about 75 amino acids to about 170 amino acids, about 75 amino acids to about 165 amino acids, about 75 amino acids to about 160 amino acids, about 75 amino acids to about 155 amino acids, about 75 amino acids to about 150 amino acids, about 75 amino acids to about 145 amino acids, about 75 amino acids to about 140 amino acids, about 75 amino acids to about 135 amino acids, about 75 amino acids to about 130 amino acids, about 75 amino acids to about 125 amino acids, about 75 amino acids to about 120 amino acids, about 75 amino acids to about 115 amino acids, about 75 amino acids to about 110 amino acids, about 75 amino acids to about 105 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 280 amino acids, about 80 amino acids to about 260 amino acids, about 80 amino acids to about 240 amino acids, about 80 amino acids to about 220 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 80 amino acids to about 85 amino acids, about 85 amino acids to about 1000 amino acids, about 85 amino acids to about 950 amino acids, about 85 amino acids to about 900 amino acids, about 85 amino acids to about 850 amino acids, about 85 amino acids to about 800 amino acids, about 85 amino acids to about 750 amino acids, about 85 amino acids to about 700 amino acids, about 85 amino acids to about 650 amino acids, about 85 amino acids to about 600 amino acids, about 85 amino acids to about 550 amino acids, about 85 amino acids to about 500 amino acids, about 85 amino acids to about 450 amino acids, about 85 amino acids to about 400 amino acids, about 85 amino acids to about 350 amino acids, about 85 amino acids to about 300 amino acids, about 85 amino acids to about 280 amino acids, about 85 amino acids to about 260 amino acids, about 85 amino acids to about 240 amino acids, about 85 amino acids to about 220 amino acids, about 85 amino acids to about 200 amino acids, about 85 amino acids to about 195 amino acids, about 85 amino acids to about 190 amino acids, about 85 amino acids to about 185 amino acids, about 85 amino acids to about 180 amino acids, about 85 amino acids to about 175 amino acids, about 85 amino acids to about 170 amino acids, about 85 amino acids to about 165 amino acids, about 85 amino acids to about 160 amino acids, about 85 amino acids to about 155 amino acids, about 85 amino acids to about 150 amino acids, about 85 amino acids to about 145 amino acids, about 85 amino acids to about 140 amino acids, about 85 amino acids to about 135 amino acids, about 85 amino acids to about 130 amino acids, about 85 amino acids to about 125 amino acids, about 85 amino acids to about 120 amino acids, about 85 amino acids to about 115 amino acids, about 85 amino acids to about 110 amino acids, about 85 amino acids to about 105 amino acids, about 85 amino acids to about 100 amino acids, about 85 amino acids to about 95 amino acids, about 85 amino acids to about 90 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 280 amino acids, about 90 amino acids to about 260 amino acids, about 90 amino acids to about 240 amino acids, about 90 amino acids to about 220 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 90 amino acids to about 95 amino acids, about 95 amino acids to about 1000 amino acids, about 95 amino acids to about 950 amino acids, about 95 amino acids to about 900 amino acids, about 95 amino acids to about 850 amino acids, about 95 amino acids to about 800 amino acids, about 95 amino acids to about 750 amino acids, about 95 amino acids to about 700 amino acids, about 95 amino acids to about 650 amino acids, about 95 amino acids to about 600 amino acids, about 95 amino acids to about 550 amino acids, about 95 amino acids to about 500 amino acids, about 95 amino acids to about 450 amino acids, about 95 amino acids to about 400 amino acids, about 95 amino acids to about 350 amino acids, about 95 amino acids to about 300 amino acids, about 95 amino acids to about 280 amino acids, about 95 amino acids to about 260 amino acids, about 95 amino acids to about 240 amino acids, about 95 amino acids to about 220 amino acids, about 95 amino acids to about 200 amino acids, about 95 amino acids to about 195 amino acids, about 95 amino acids to about 190 amino acids, about 95 amino acids to about 185 amino acids, about 95 amino acids to about 180 amino acids, about 95 amino acids to about 175 amino acids, about 95 amino acids to about 170 amino acids, about 95 amino acids to about 165 amino acids, about 95 amino acids to about 160 amino acids, about 95 amino acids to about 155 amino acids, about 95 amino acids to about 150 amino acids, about 95 amino acids to about 145 amino acids, about 95 amino acids to about 140 amino acids, about 95 amino acids to about 135 amino acids, about 95 amino acids to about 130 amino acids, about 95 amino acids to about 125 amino acids, about 95 amino acids to about 120 amino acids, about 95 amino acids to about 115 amino acids, about 95 amino acids to about 110 amino acids, about 95 amino acids to about 105 amino acids, about 95 amino acids to about 100 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 100 amino acids to about 105 amino acids, about 105 amino acids to about 1000 amino acids, about 105 amino acids to about 950 amino acids, about 105 amino acids to about 900 amino acids, about 105 amino acids to about 850 amino acids, about 105 amino acids to about 800 amino acids, about 105 amino acids to about 750 amino acids, about 105 amino acids to about 700 amino acids, about 105 amino acids to about 650 amino acids, about 105 amino acids to about 600 amino acids, about 105 amino acids to about 550 amino acids, about 105 amino acids to about 500 amino acids, about 105 amino acids to about 450 amino acids, about 105 amino acids to about 400 amino acids, about 105 amino acids to about 350 amino acids, about 105 amino acids to about 300 amino acids, about 105 amino acids to about 280 amino acids, about 105 amino acids to about 260 amino acids, about 105 amino acids to about 240 amino acids, about 105 amino acids to about 220 amino acids, about 105 amino acids to about 200 amino acids, about 105 amino acids to about 195 amino acids, about 105 amino acids to about 190 amino acids, about 105 amino acids to about 185 amino acids, about 105 amino acids to about 180 amino acids, about 105 amino acids to about 175 amino acids, about 105 amino acids to about 170 amino acids, about 105 amino acids to about 165 amino acids, about 105 amino acids to about 160 amino acids, about 105 amino acids to about 155 amino acids, about 105 amino acids to about 150 amino acids, about 105 amino acids to about 145 amino acids, about 105 amino acids to about 140 amino acids, about 105 amino acids to about 135 amino acids, about 105 amino acids to about 130 amino acids, about 105 amino acids to about 125 amino acids, about 105 amino acids to about 120 amino acids, about 105 amino acids to about 115 amino acids, about 105 amino acids to about 110 amino acids, about 110 amino acids to about 1000 amino acids, about 110 amino acids to about 950 amino acids, about 110 amino acids to about 900 amino acids, about 110 amino acids to about 850 amino acids, about 110 amino acids to about 800 amino acids, about 110 amino acids to about 750 amino acids, about 110 amino acids to about 700 amino acids, about 110 amino acids to about 650 amino acids, about 110 amino acids to about 600 amino acids, about 110 amino acids to about 550 amino acids, about 110 amino acids to about 500 amino acids, about 110 amino acids to about 450 amino acids, about 110 amino acids to about 400 amino acids, about 110 amino acids to about 350 amino acids, about 110 amino acids to about 300 amino acids, about 110 amino acids to about 280 amino acids, about 110 amino acids to about 260 amino acids, about 110 amino acids to about 240 amino acids, about 110 amino acids to about 220 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 1000 amino acids, about 115 amino acids to about 950 amino acids, about 115 amino acids to about 900 amino acids, about 115 amino acids to about 850 amino acids, about 115 amino acids to about 800 amino acids, about 115 amino acids to about 750 amino acids, about 115 amino acids to about 700 amino acids, about 115 amino acids to about 650 amino acids, about 115 amino acids to about 600 amino acids, about 115 amino acids to about 550 amino acids, about 115 amino acids to about 500 amino acids, about 115 amino acids to about 450 amino acids, about 115 amino acids to about 400 amino acids, about 115 amino acids to about 350 amino acids, about 115 amino acids to about 300 amino acids, about 115 amino acids to about 280 amino acids, about 115 amino acids to about 260 amino acids, about 115 amino acids to about 240 amino acids, about 115 amino acids to about 220 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 1000 amino acids, about 120 amino acids to about 950 amino acids, about 120 amino acids to about 900 amino acids, about 120 amino acids to about 850 amino acids, about 120 amino acids to about 800 amino acids, about 120 amino acids to about 750 amino acids, about 120 amino acids to about 700 amino acids, about 120 amino acids to about 650 amino acids, about 120 amino acids to about 600 amino acids, about 120 amino acids to about 550 amino acids, about 120 amino acids to about 500 amino acids, about 120 amino acids to about 450 amino acids, about 120 amino acids to about 400 amino acids, about 120 amino acids to about 350 amino acids, about 120 amino acids to about 300 amino acids, about 120 amino acids to about 280 amino acids, about 120 amino acids to about 260 amino acids, about 120 amino acids to about 240 amino acids, about 120 amino acids to about 220 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 1000 amino acids, about 125 amino acids to about 950 amino acids, about 125 amino acids to about 900 amino acids, about 125 amino acids to about 850 amino acids, about 125 amino acids to about 800 amino acids, about 125 amino acids to about 750 amino acids, about 125 amino acids to about 700 amino acids, about 125 amino acids to about 650 amino acids, about 125 amino acids to about 600 amino acids, about 125 amino acids to about 550 amino acids, about 125 amino acids to about 500 amino acids, about 125 amino acids to about 450 amino acids, about 125 amino acids to about 400 amino acids, about 125 amino acids to about 350 amino acids, about 125 amino acids to about 300 amino acids, about 125 amino acids to about 280 amino acids, about 125 amino acids to about 260 amino acids, about 125 amino acids to about 240 amino acids, about 125 amino acids to about 220 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 1000 amino acids, about 130 amino acids to about 950 amino acids, about 130 amino acids to about 900 amino acids, about 130 amino acids to about 850 amino acids, about 130 amino acids to about 800 amino acids, about 130 amino acids to about 750 amino acids, about 130 amino acids to about 700 amino acids, about 130 amino acids to about 650 amino acids, about 130 amino acids to about 600 amino acids, about 130 amino acids to about 550 amino acids, about 130 amino acids to about 500 amino acids, about 130 amino acids to about 450 amino acids, about 130 amino acids to about 400 amino acids, about 130 amino acids to about 350 amino acids, about 130 amino acids to about 300 amino acids, about 130 amino acids to about 280 amino acids, about 130 amino acids to about 260 amino acids, about 130 amino acids to about 240 amino acids, about 130 amino acids to about 220 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 1000 amino acids, about 135 amino acids to about 950 amino acids, about 135 amino acids to about 900 amino acids, about 135 amino acids to about 850 amino acids, about 135 amino acids to about 800 amino acids, about 135 amino acids to about 750 amino acids, about 135 amino acids to about 700 amino acids, about 135 amino acids to about 650 amino acids, about 135 amino acids to about 600 amino acids, about 135 amino acids to about 550 amino acids, about 135 amino acids to about 500 amino acids, about 135 amino acids to about 450 amino acids, about 135 amino acids to about 400 amino acids, about 135 amino acids to about 350 amino acids, about 135 amino acids to about 300 amino acids, about 135 amino acids to about 280 amino acids, about 135 amino acids to about 260 amino acids, about 135 amino acids to about 240 amino acids, about 135 amino acids to about 220 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 1000 amino acids, about 140 amino acids to about 950 amino acids, about 140 amino acids to about 900 amino acids, about 140 amino acids to about 850 amino acids, about 140 amino acids to about 800 amino acids, about 140 amino acids to about 750 amino acids, about 140 amino acids to about 700 amino acids, about 140 amino acids to about 650 amino acids, about 140 amino acids to about 600 amino acids, about 140 amino acids to about 550 amino acids, about 140 amino acids to about 500 amino acids, about 140 amino acids to about 450 amino acids, about 140 amino acids to about 400 amino acids, about 140 amino acids to about 350 amino acids, about 140 amino acids to about 300 amino acids, about 140 amino acids to about 280 amino acids, about 140 amino acids to about 260 amino acids, about 140 amino acids to about 240 amino acids, about 140 amino acids to about 220 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 1000 amino acids, about 145 amino acids to about 950 amino acids, about 145 amino acids to about 900 amino acids, about 145 amino acids to about 850 amino acids, about 145 amino acids to about 800 amino acids, about 145 amino acids to about 750 amino acids, about 145 amino acids to about 700 amino acids, about 145 amino acids to about 650 amino acids, about 145 amino acids to about 600 amino acids, about 145 amino acids to about 550 amino acids, about 145 amino acids to about 500 amino acids, about 145 amino acids to about 450 amino acids, about 145 amino acids to about 400 amino acids, about 145 amino acids to about 350 amino acids, about 145 amino acids to about 300 amino acids, about 145 amino acids to about 280 amino acids, about 145 amino acids to about 260 amino acids, about 145 amino acids to about 240 amino acids, about 145 amino acids to about 220 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 1000 amino acids, about 155 amino acids to about 950 amino acids, about 155 amino acids to about 900 amino acids, about 155 amino acids to about 850 amino acids, about 155 amino acids to about 800 amino acids, about 155 amino acids to about 750 amino acids, about 155 amino acids to about 700 amino acids, about 155 amino acids to about 650 amino acids, about 155 amino acids to about 600 amino acids, about 155 amino acids to about 550 amino acids, about 155 amino acids to about 500 amino acids, about 155 amino acids to about 450 amino acids, about 155 amino acids to about 400 amino acids, about 155 amino acids to about 350 amino acids, about 155 amino acids to about 300 amino acids, about 155 amino acids to about 280 amino acids, about 155 amino acids to about 260 amino acids, about 155 amino acids to about 240 amino acids, about 155 amino acids to about 220 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 1000 amino acids, about 160 amino acids to about 950 amino acids, about 160 amino acids to about 900 amino acids, about 160 amino acids to about 850 amino acids, about 160 amino acids to about 800 amino acids, about 160 amino acids to about 750 amino acids, about 160 amino acids to about 700 amino acids, about 160 amino acids to about 650 amino acids, about 160 amino acids to about 600 amino acids, about 160 amino acids to about 550 amino acids, about 160 amino acids to about 500 amino acids, about 160 amino acids to about 450 amino acids, about 160 amino acids to about 400 amino acids, about 160 amino acids to about 350 amino acids, about 160 amino acids to about 300 amino acids, about 160 amino acids to about 280 amino acids, about 160 amino acids to about 260 amino acids, about 160 amino acids to about 240 amino acids, about 160 amino acids to about 220 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 1000 amino acids, about 165 amino acids to about 950 amino acids, about 165 amino acids to about 900 amino acids, about 165 amino acids to about 850 amino acids, about 165 amino acids to about 800 amino acids, about 165 amino acids to about 750 amino acids, about 165 amino acids to about 700 amino acids, about 165 amino acids to about 650 amino acids, about 165 amino acids to about 600 amino acids, about 165 amino acids to about 550 amino acids, about 165 amino acids to about 500 amino acids, about 165 amino acids to about 450 amino acids, about 165 amino acids to about 400 amino acids, about 165 amino acids to about 350 amino acids, about 165 amino acids to about 300 amino acids, about 165 amino acids to about 280 amino acids, about 165 amino acids to about 260 amino acids, about 165 amino acids to about 240 amino acids, about 165 amino acids to about 220 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 1000 amino acids, about 170 amino acids to about 950 amino acids, about 170 amino acids to about 900 amino acids, about 170 amino acids to about 850 amino acids, about 170 amino acids to about 800 amino acids, about 170 amino acids to about 750 amino acids, about 170 amino acids to about 700 amino acids, about 170 amino acids to about 650 amino acids, about 170 amino acids to about 600 amino acids, about 170 amino acids to about 550 amino acids, about 170 amino acids to about 500 amino acids, about 170 amino acids to about 450 amino acids, about 170 amino acids to about 400 amino acids, about 170 amino acids to about 350 amino acids, about 170 amino acids to about 300 amino acids, about 170 amino acids to about 280 amino acids, about 170 amino acids to about 260 amino acids, about 170 amino acids to about 240 amino acids, about 170 amino acids to about 220 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 1000 amino acids, about 175 amino acids to about 950 amino acids, about 175 amino acids to about 900 amino acids, about 175 amino acids to about 850 amino acids, about 175 amino acids to about 800 amino acids, about 175 amino acids to about 750 amino acids, about 175 amino acids to about 700 amino acids, about 175 amino acids to about 650 amino acids, about 175 amino acids to about 600 amino acids, about 175 amino acids to about 550 amino acids, about 175 amino acids to about 500 amino acids, about 175 amino acids to about 450 amino acids, about 175 amino acids to about 400 amino acids, about 175 amino acids to about 350 amino acids, about 175 amino acids to about 300 amino acids, about 175 amino acids to about 280 amino acids, about 175 amino acids to about 260 amino acids, about 175 amino acids to about 240 amino acids, about 175 amino acids to about 220 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 1000 amino acids, about 180 amino acids to about 950 amino acids, about 180 amino acids to about 900 amino acids, about 180 amino acids to about 850 amino acids, about 180 amino acids to about 800 amino acids, about 180 amino acids to about 750 amino acids, about 180 amino acids to about 700 amino acids, about 180 amino acids to about 650 amino acids, about 180 amino acids to about 600 amino acids, about 180 amino acids to about 550 amino acids, about 180 amino acids to about 500 amino acids, about 180 amino acids to about 450 amino acids, about 180 amino acids to about 400 amino acids, about 180 amino acids to about 350 amino acids, about 180 amino acids to about 300 amino acids, about 180 amino acids to about 280 amino acids, about 180 amino acids to about 260 amino acids, about 180 amino acids to about 240 amino acids, about 180 amino acids to about 220 amino acids, about 180 amino acids to about 200 amino acids, about 180 amino acids to about 195 amino acids, about 180 amino acids to about 190 amino acids, about 180 amino acids to about 185 amino acids, about 185 amino acids to about 1000 amino acids, about 185 amino acids to about 950 amino acids, about 185 amino acids to about 900 amino acids, about 185 amino acids to about 850 amino acids, about 185 amino acids to about 800 amino acids, about 185 amino acids to about 750 amino acids, about 185 amino acids to about 700 amino acids, about 185 amino acids to about 650 amino acids, about 185 amino acids to about 600 amino acids, about 185 amino acids to about 550 amino acids, about 185 amino acids to about 500 amino acids, about 185 amino acids to about 450 amino acids, about 185 amino acids to about 400 amino acids, about 185 amino acids to about 350 amino acids, about 185 amino acids to about 300 amino acids, about 185 amino acids to about 280 amino acids, about 185 amino acids to about 260 amino acids, about 185 amino acids to about 240 amino acids, about 185 amino acids to about 220 amino acids, about 185 amino acids to about 200 amino acids, about 185 amino acids to about 195 amino acids, about 185 amino acids to about 190 amino acids, about 190 amino acids to about 1000 amino acids, about 190 amino acids to about 950 amino acids, about 190 amino acids to about 900 amino acids, about 190 amino acids to about 850 amino acids, about 190 amino acids to about 800 amino acids, about 190 amino acids to about 750 amino acids, about 190 amino acids to about 700 amino acids, about 190 amino acids to about 650 amino acids, about 190 amino acids to about 600 amino acids, about 190 amino acids to about 550 amino acids, about 190 amino acids to about 500 amino acids, about 190 amino acids to about 450 amino acids, about 190 amino acids to about 400 amino acids, about 190 amino acids to about 350 amino acids, about 190 amino acids to about 300 amino acids, about 190 amino acids to about 280 amino acids, about 190 amino acids to about 260 amino acids, about 190 amino acids to about 240 amino acids, about 190 amino acids to about 220 amino acids, about 190 amino acids to about 200 amino acids, about 190 amino acids to about 195 amino acids, about 195 amino acids to about 1000 amino acids, about 195 amino acids to about 950 amino acids, about 195 amino acids to about 900 amino acids, about 195 amino acids to about 850 amino acids, about 195 amino acids to about 800 amino acids, about 195 amino acids to about 750 amino acids, about 195 amino acids to about 700 amino acids, about 195 amino acids to about 650 amino acids, about 195 amino acids to about 600 amino acids, about 195 amino acids to about 550 amino acids, about 195 amino acids to about 500 amino acids, about 195 amino acids to about 450 amino acids, about 195 amino acids to about 400 amino acids, about 195 amino acids to about 350 amino acids, about 195 amino acids to about 300 amino acids, about 195 amino acids to about 280 amino acids, about 195 amino acids to about 260 amino acids, about 195 amino acids to about 240 amino acids, about 195 amino acids to about 220 amino acids, about 195 amino acids to about 200 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 450 amino acids, about 220 amino acids to about 400 amino acids, about 220 amino acids to about 350 amino acids, about 220 amino acids to about 300 amino acids, about 220 amino acids to about 280 amino acids, about 220 amino acids to about 260 amino acids, about 220 amino acids to about 240 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 450 amino acids, about 240 amino acids to about 400 amino acids, about 240 amino acids to about 350 amino acids, about 240 amino acids to about 300 amino acids, about 240 amino acids to about 280 amino acids, about 240 amino acids to about 260 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 450 amino acids, about 260 amino acids to about 400 amino acids, about 260 amino acids to about 350 amino acids, about 260 amino acids to about 300 amino acids, about 260 amino acids to about 280 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 450 amino acids, about 280 amino acids to about 400 amino acids, about 280 amino acids to about 350 amino acids, about 280 amino acids to about 300 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1000 amino acids, about 850 amino acids to about 950 amino acids, about 850 amino acids to about 900 amino acids, about 900 amino acids to about 1000 amino acids, about 900 amino acids to about 950 amino acids, or about 950 amino acids to about 1000 amino acids.

Any of the target-binding domains described herein can bind to its target with a dissociation equilibrium constant (K_{D}) of less than 1×10⁻⁷M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the antigen-binding protein construct provided herein can bind to an identifying antigen with a K_{D} of about 1×10⁻³ M to about 1×10⁻⁵ M, about 1×10⁻⁴ M to about 1×10⁻⁶M, about 1×10⁻⁵ M to about 1×10⁻⁷ M, about 1×10⁻⁶ M to about 1×10⁻⁸ M, about 1×10⁻⁷ M to about 1×10⁻⁹M, about 1×10⁻⁸ M to about 1×10⁻¹⁰ M, or about 1×10⁻⁹ M to about 1×10⁻¹¹ M (inclusive).

Any of the target-binding domains described herein can bind to its target with a K_{D} of between about 1 pM to about 30 nM (e.g., about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 pM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 30 nM, about 30 pM to about 25 nM, about 30 pM to about 30 nM, about 30 pM to about 15 nM, about 30 pM to about 10 nM, about 30 PM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 PM to about 650 pM, about 30 pM to about 600 pM, about 30 pM to about 550 pM, about 30 pM to about 500 pM, about 30 pM to about 450 pM, about 30 pM to about 400 pM, about 30 pM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 30 nM, about 50 PM to about 25 nM, about 50 PM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 30 nM, about 60 pM to about 25 nM, about 60 pM to about 30 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 30 nM, about 70 pM to about 25 nM, about 70 pM to about 30 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 30 nM, about 80 pM to about 25 nM, about 80 pM to about 30 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 30 nM, about 300 pM to about 15 nM, about 300 pM to about 10 nM, about 300 PM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 30 nM, about 400 pM to about 25 nM, about 400 pM to about 30 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 30 nM, about 500 pM to about 25 nM, about 500 pM to about 30 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 30 nM, about 600 pM to about 25 nM, about 600 pM to about 30 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 30 nM, about 700 pM to about 25 nM, about 700 pM to about 30 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 30 nM, about 800 pM to about 25 nM, about 800 pM to about 30 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 30 nM, about 900 pM to about 25 nM, about 900 pM to about 30 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 30 nM, about 5 nM to about 25 nM, about 5 nM to about 20 nM, about 5 nM to about 15 nM, about 5 nM to about 10 nM, about 10 nM to about 30 nM, about 10 nM to about 25 nM, about 10 nM to about 20 nM, about 10 nM to about 15 nM, about 15 nM to about 30 nM, about 15 nM to about 25 nM, about 15 nM to about 20 nM, about 20 nM to about 30 nM, and about 20 nM to about 25 nM).

Any of the target-binding domains described herein can bind to its target with a K_{D} of between about 1 nM to about 10 nM (e.g., about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, and about 9 nM to about 10 nM).

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Soluble Interleukin or Cytokine Protein

In some embodiments of any of the single-chain chimeric polypeptides described herein, one or both of the first target-binding domain and the second target-binding domain can be a soluble interleukin protein, soluble cytokine protein, or soluble cell surface protein. In some embodiments, the soluble interleukin protein or soluble cytokine protein, is selected from the group of: IL-2, IL-3, IL-4, IL-7, IL-8, IL-10, IL-12, IL-15, IL-17, IL-18, IL-21, PDGF-DD, SCF, FLT3L. Non-limiting examples of soluble IL-7 and IL-21 are provided below.

In some embodiments, a target-binding domain can comprise a sequence that is at least 80%, 85%, 90%, 94%, 95%, 96%, 98%, or 100% identical to SEQ ID NO: 4 or 5. In some embodiments, a target-binding domain can be encoded by a sequence that is at least 80%, 85%, 90%, 94%, 95%, 96%, 98%, or 100% identical to SEQ ID NO: 6 or 11.

Human Soluble IL-7
(SEQ ID NO: 4)
DCDIEGKDGKQYESVLMVSIDQLLDSMKEIGSNCLNNEFNFFKRHIC
DANKEGMFLFRAARKLRQFLKMNSTGDFDLHLLKVSEGTTILLNCTG
QVKGRKPAALGEAQPTKSLEENKSLKEQKKLNDLCFLKRLLQEIKTC
WNKILMGTKEH
Nucleic Acid Encoding Human Soluble IL-7
(SEQ ID NO: 64)
GATTGTGATATTGAAGGTAAAGATGGCAAACAATATGAGAGTGTTCT
AATGGTCAGCATCGATCAATTATTGGACAGCATGAAAGAAATTGGTA
GCAATTGCCTGAATAATGAATTTAACTTTTTTAAAAGACATATCTGT
GATGCTAATAAGGAAGGTATGTTTTTATTCCGTGCTGCTCGCAAGTT
GAGGCAATTTCTTAAAATGAATAGCACTGGTGATTTTGATCTCCACT
TATTAAAAGTTTCAGAAGGCACAACAATACTGTTGAACTGCACTGGC
CAGGTTAAAGGAAGAAAACCAGCTGCCCTGGGTGAAGCCCAACCAAC
AAAGAGTTTGGAAGAAAATAAATCTTTAAAGGAACAGAAAAAACTGA
ATGACTTGTGTTTCCTAAAGAGACTATTACAAGAGATAAAAACTTGT
TGGAATAAAATTTTGATGGGCACTAAAGAACAC
Human Soluble IL-21
(SEQ ID NO: 5)
QGQDRHMIRMRQLIDIVDQLKNYVNDLVPEFLPAPEDVETNCEWSAF
SCFQKAQLKSANTGNNERIINVSIKKLKRKPPSTNAGRRQKHRLTCP
SCDSYEKKPPKEFLERFKSLLQKMIHQHLSSRTHGSEDS
Nucleic Acid Encoding Human Soluble IL-21
(SEQ ID NO: 6)
CAAGGTCAAGATCGCCACATGATTAGAATGCGTCAACTTATAGATAT
TGTTGATCAGCTGAAAAATTATGTGAATGACTTGGTCCCTGAATTTC
TGCCAGCTCCAGAAGATGTAGAGACAAACTGTGAGTGGTCAGCTTTT
TCCTGTTTTCAGAAGGCCCAACTAAAGTCAGCAAATACAGGAAACAA
TGAAAGGATAATCAATGTATCAATTAAAAAGCTGAAGAGGAAACCAC
CTTCCACAAATGCAGGGAGAAGACAGAAACACAGACTAACATGCCCT
TCATGTGATTCTTATGAGAAAAAACCACCCAAAGAATTCCTAGAAAG
ATTCAAATCACTTCTCCAAAAGATGATTCATCAGCATCTGTCCTCTA
GAACACACGGAAGTGAAGATTCC
Nucleic Acid Encoding Human Soluble IL-21
(SEQ ID NO: 11)
CAGGGCCAGGACAGGCACATGATCCGGATGAGGCAGCTCATCGACAT
CGTCGACCAGCTGAAGAACTACGTGAACGACCTGGTGCCCGAGTTTC
TGCCTGCCCCCGAGGACGTGGAGACCAACTGCGAGTGGTCCGCCTTC
TCCTGCTTTCAGAAGGCCCAGCTGAAGTCCGCCAACACCGGCAACAA
CGAGCGGATCATCAACGTGAGCATCAAGAAGCTGAAGCGGAAGCCTC
CCTCCACAAACGCCGGCAGGAGGCAGAAGCACAGGCTGACCTGCCCC
AGCTGTGACTCCTACGAGAAGAAGCCCCCCAAGGAGTTCCTGGAGAG
GTTCAAGTCCCTGCTGCAGAAGATGATCCATCAGCACCTGTCCTCCA
GGACCCACGGCTCCGAGGACTCC
Mouse Soluble IL-21
(SEQ ID NO: 132)
HKSSPQGPDRLLIRLRHLIDIVEQLKIYENDLDPELLSAPQDVKGHC
EHAAFACFQKAKLKPSNPGNNKTFIIDLVAQLRRRLPARRGGKKQKH
IAKCPSCDSYEKRTPKEFLERLKWLLQKMIHQHLS
Nucleic Acid Encoding Mouse Soluble IL-21
(SEQ ID NO: 133)
CATAAATCAAGCCCCCAAGGGCCAGATCGCCTCCTGATTAGACTTCG
TCACCTTATTGACATTGTTGAACAGCTGAAAATCTATGAAAATGACT
TGGATCCTGAACTTCTATCAGCTCCACAAGATGTAAAGGGGCACTGT
GAGCATGCAGCTTTTGCCTGTTTTCAGAAGGCCAAACTCAAGCCATC
AAACCCTGGAAACAATAAGACATTCATCATTGACCTCGTGGCCCAGC
TCAGGAGGAGGCTGCCTGCCAGGAGGGGAGGAAAGAAACAGAAGCAC
ATAGCTAAATGCCCTTCCTGTGATTCGTATGAGAAAAGGACACCCAA
AGAATTCCTAGAAAGACTAAAATGGCTCCTTCAAAAGATGATTCATC
AGCATCTCTCC

In some embodiments, a soluble IL-7 protein can include a sequence that is at least 70% identical (e.g., at least 75% identical, at least 80% identical, at least 85% identical, at least 90% identical, at least 95% identical, at least 99% identical, or 100% identical) to SEQ ID NO: 4.

In some embodiments, a soluble IL-21 protein can include a sequence that is at least 70% identical (e.g., at least 75% identical, at least 80% identical, at least 85% identical, at least 90% identical, at least 95% identical, at least 99% identical, or 100% identical) to SEQ ID NO: 5 or SEQ ID NO: 132. In some embodiments, a soluble IL-21 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 6 or 11.

Non-limiting examples of additional soluble interleukin proteins or soluble cytokine proteins are provided below.

Human Soluble IL-2
(SEQ ID NO: 12)
APTSSSTKKTQLQLEHLLLDLQMILNGINNYKNPKLTRMLTFKFYMP
KKATELKHLQCLEEELKPLEEVLNLAQSKNFHLRPRDLISNINVIVL
ELKGSETTFMCEYADETATIVEFLNRWITFCQSIISTLT
Human Soluble IL-3
(SEQ ID NO: 13)
APMTQTTPLKTSWVNCSNMIDEIITHLKQPPLPLLDFNNLNGEDQDI
LMENNLRRPNLEAFNRAVKSLQNASAIESILKNLLPCLPLATAAPTR
HPIHIKDGDWNEFRRKLTFYLKTLENAQAQQTTLSLAIF
Human Soluble IL-8
(SEQ ID NO: 14)
EGAVLPRSAKELRCQCIKTYSKPFHPKFIKELRVIESGPHCANTEII
VKLSDGRELCLDPKENWVQRVVEKFLKRAENS
Human Soluble IL-10
(SEQ ID NO: 15)
SPGQGTQSENSCTHFPGNLPNMLRDLRDAFSRVKTFFQMKDQLDNLL
LKESLLEDFKGYLGCQALSEMIQFYLEEVMPQAENQDPDIKAHVNSL
GENLKTLRLRLRRCHRFLPCENKSKAVEQVKNAFNKLQEKGIYKAMS
EFDIFINYIEAYMTMKIRN
Human Soluble IL-12β (p40)
(SEQ ID NO: 16)
IWELKKDVYVVELDWYPDAPGEMVVLTCDTPEEDGITWTLDQSSEVL
GSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSS
DPQGVTCGAATLSAERVRGDNKEYEYSVECQEDSACPAAEESLPIEV
MVDAVHKLKYENYTSSFFIRDIIKPDPPKNLQLKPLKNSRQVEVSWE
YPDTWSTPHSYFSLTFCVQVQGKSKREKKDRVFTDKTSATVICRKNA
SISVRAQDRYYSSSWSEWASVPCS
Nucleic Acid Encoding Human Soluble IL-12β (p40)
(SEQ ID NO: 17)
ATTTGGGAACTGAAGAAGGACGTCTACGTGGTCGAACTGGACTGGTA
TCCCGATGCTCCCGGCGAAATGGTGGTGCTCACTTGTGACACCCCCG
AAGAAGACGGCATCACTTGGACCCTCGATCAGAGCAGCGAGGTGCTG
GGCTCCGGAAAGACCCTCACAATCCAAGTTAAGGAGTTCGGAGACGC
TGGCCAATACACATGCCACAAGGGAGGCGAGGTGCTCAGCCATTCCT
TATTATTATTACACAAGAAGGAAGACGGAATCTGGTCCACCGACATT
TTAAAAGATCAGAAGGAGCCCAAGAATAAGACCTTTTTAAGGTGTGA
GGCCAAAAACTACAGCGGTCGTTTCACTTGTTGGTGGCTGACCACCA
TTTCCACCGATTTAACCTTCTCCGTGAAAAGCAGCCGGGGAAGCTCC
GACCCTCAAGGTGTGACATGTGGAGCCGCTACCCTCAGCGCTGAGAG
GGTTCGTGGCGATAACAAGGAATACGAGTACAGCGTGGAGTGCCAAG
AAGATAGCGCTTGTCCCGCTGCCGAAGAATCTTTACCCATTGAGGTG
ATGGTGGACGCCGTGCACAAACTCAAGTACGAGAACTACACCTCCTC
CTTCTTTATCCGGGACATCATTAAGCCCGATCCTCCTAAGAATTTAC
AGCTGAAGCCTCTCAAAAATAGCCGGCAAGTTGAGGTCTCTTGGGAA
TATCCCGACACTTGGAGCACACCCCACAGCTACTTCTCTTTAACCTT
TTGTGTGCAAGTTCAAGGTAAAAGCAAGCGGGAGAAGAAAGACCGGG
TGTTTACCGACAAAACCAGCGCCACCGTCATCTGTCGGAAGAACGCC
TCCATCAGCGTGAGGGCTCAAGATCGTTATTACTCCAGCAGCTGGTC
CGAGTGGGCCAGCGTGCCTTGTTCC
Human Soluble IL-12α (p35)
(SEQ ID NO: 18)
RNLPVATPDPGMFPCLHHSQNLLRAVSNMLQKARQTLEFYPCTSEEI
DHEDITKDKTSTVEACLPLELTKNESCLNSRETSFITNGSCLASRKT
SFMMALCLSSIYEDLKMYQVEFKTMNAKLLMDPKRQIFLDQNMLAVI
DELMQALNFNSETVPQKSSLEEPDFYKTKIKLCILLHAFRIRAVTID
RVMSYLNAS
Nucleic Acid Encoding Human Soluble IL-12α (p35)
(SEQ ID NO: 19)
CGTAACCTCCCCGTGGCTACCCCCGATCCCGGAATGTTCCCTTGTTT
ACACCACAGCCAGAATTTACTGAGGGCCGTGAGCAACATGCTGCAGA
AAGCTAGGCAGACTTTAGAATTTTACCCTTGCACCAGCGAGGAGATC
GACCATGAAGATATCACCAAGGACAAGACATCCACCGTGGAGGCTTG
TTTACCTCTGGAGCTGACAAAGAACGAGTCTTGTCTCAACTCTCGTG
AAACCAGCTTCATCACAAATGGCTCTTGTTTAGCTTCCCGGAAGACC
TCCTTTATGATGGCTTTATGCCTCAGCTCCATCTACGAGGATTTAAA
GATGTACCAAGTGGAGTTCAAGACCATGAACGCCAAGCTGCTCATGG
ACCCTAAACGGCAGATCTTTTTAGACCAGAACATGCTGGCTGTGATT
GATGAGCTGATGCAAGCTTTAAACTTCAACTCCGAGACCGTCCCTCA
GAAGTCCTCCCTCGAGGAGCCCGATTTTTACAAGACAAAGATCAAAC
TGTGCATTTTACTCCACGCCTTTAGGATCCGGGCCGTGACCATTGAC
CGGGTCATGAGCTATTTAAACGCCAGC
Exemplary Human Soluble IL-12
(SEQ ID NO: 20)
IWELKKDVYVVELDWYPDAPGEMVVLTCDTPEEDGITWTLDQSSEVL
GSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSS
DPQGVTCGAATLSAERVRGDNKEYEYSVECQEDSACPAAEESLPIEV
MVDAVHKLKYENYTSSFFIRDIIKPDPPKNLQLKPLKNSRQVEVSWE
YPDTWSTPHSYFSLTFCVQVQGKSKREKKDRVFTDKTSATVICRKNA
SISVRAQDRYYSSSWSEWASVPCSGGGGSGGGGSGGGGSRNLPVATP
DPGMFPCLHHSQNLLRAVSNMLQKARQTLEFYPCTSEEIDHEDITKD
KTSTVEACLPLELTKNESCLNSRETSFITNGSCLASRKTSFMMALCL
SSIYEDLKMYQVEFKTMNAKLLMDPKRQIFLDQNMLAVIDELMQALN
FNSETVPQKSSLEEPDFYKTKIKLCILLHAFRIRAVTIDRVMSYLNA
S
Nucleic Acid Encoding Exemplary Human Soluble
IL-12
(SEQ ID NO: 21)
ATTTGGGAACTGAAGAAGGACGTCTACGTGGTCGAACTGGACTGGTA
TCCCGATGCTCCCGGCGAAATGGTGGTGCTCACTTGTGACACCCCCG
AAGAAGACGGCATCACTTGGACCCTCGATCAGAGCAGCGAGGTGCTG
GGCTCCGGAAAGACCCTCACAATCCAAGTTAAGGAGTTCGGAGACGC
TGGCCAATACACATGCCACAAGGGAGGCGAGGTGCTCAGCCATTCCT
TATTATTATTACACAAGAAGGAAGACGGAATCTGGTCCACCGACATT
TTAAAAGATCAGAAGGAGCCCAAGAATAAGACCTTTTTAAGGTGTGA
GGCCAAAAACTACAGCGGTCGTTTCACTTGTTGGTGGCTGACCACCA
TTTCCACCGATTTAACCTTCTCCGTGAAAAGCAGCCGGGGAAGCTCC
GACCCTCAAGGTGTGACATGTGGAGCCGCTACCCTCAGCGCTGAGAG
GGTTCGTGGCGATAACAAGGAATACGAGTACAGCGTGGAGTGCCAAG
AAGATAGCGCTTGTCCCGCTGCCGAAGAATCTTTACCCATTGAGGTG
ATGGTGGACGCCGTGCACAAACTCAAGTACGAGAACTACACCTCCTC
CTTCTTTATCCGGGACATCATTAAGCCCGATCCTCCTAAGAATTTAC
AGCTGAAGCCTCTCAAAAATAGCCGGCAAGTTGAGGTCTCTTGGGAA
TATCCCGACACTTGGAGCACACCCCACAGCTACTTCTCTTTAACCTT
TTGTGTGCAAGTTCAAGGTAAAAGCAAGCGGGAGAAGAAAGACCGGG
TGTTTACCGACAAAACCAGCGCCACCGTCATCTGTCGGAAGAACGCC
TCCATCAGCGTGAGGGCTCAAGATCGTTATTACTCCAGCAGCTGGTC
CGAGTGGGCCAGCGTGCCTTGTTCCGGCGGTGGAGGATCCGGAGGAG
GTGGCTCCGGCGGCGGAGGATCTCGTAACCTCCCCGTGGCTACCCCC
GATCCCGGAATGTTCCCTTGTTTACACCACAGCCAGAATTTACTGAG
GGCCGTGAGCAACATGCTGCAGAAAGCTAGGCAGACTTTAGAATTTT
ACCCTTGCACCAGCGAGGAGATCGACCATGAAGATATCACCAAGGAC
AAGACATCCACCGTGGAGGCTTGTTTACCTCTGGAGCTGACAAAGAA
CGAGTCTTGTCTCAACTCTCGTGAAACCAGCTTCATCACAAATGGCT
CTTGTTTAGCTTCCCGGAAGACCTCCTTTATGATGGCTTTATGCCTC
AGCTCCATCTACGAGGATTTAAAGATGTACCAAGTGGAGTTCAAGAC
CATGAACGCCAAGCTGCTCATGGACCCTAAACGGCAGATCTTTTTAG
ACCAGAACATGCTGGCTGTGATTGATGAGCTGATGCAAGCTTTAAAC
TTCAACTCCGAGACCGTCCCTCAGAAGTCCTCCCTCGAGGAGCCCGA
TTTTTACAAGACAAAGATCAAACTGTGCATTTTACTCCACGCCTTTA
GGATCCGGGCCGTGACCATTGACCGGGTCATGAGCTATTTAAACGCC
AGC
Human Soluble IL-15
(SEQ ID NO: 22)
NWVNVISDLKKIEDLIQSMHIDATLYTESDVHPSCKVTAMKCFLLEL
QVISLESGDASIHDTVENLIILANNSLSSNGNVTESGCKECEELEEK
NIKEFLQSFVHIVQMFINTS
Human Soluble IL-17
(SEQ ID NO: 23)
GITIPRNPGCPNSEDKNFPRTVMVNLNIHNRNTNTNPKRSSDYYNRS
TSPWNLHRNEDPERYPSVIWEAKCRHLGCINADGNVDYHMNSVPIQQ
EILVLRREPPHCPNSFRLEKILVSVGCTCVTPIVHHVA
Human Soluble IL-18
(SEQ ID NO: 24)
YFGKLESKLSVIRNLNDQVLFIDQGNRPLFEDMTDSDCRDNAPRTIF
IISMYKDSQPRGMAVTISVKCEKISTLSCENKIISFKEMNPPDNIKD
TKSDIIFFQRSVPGHDNKMQFESSSYEGYFLACEKERDLFKLILKKE
DELGDRSIMFTVQNED
Nucleic Acid Encoding Human Soluble IL-18
(SEQ ID NO: 25)
TACTTCGGCAAACTGGAATCCAAGCTGAGCGTGATCCGGAATTTAAA
CGACCAAGTTCTGTTTATCGATCAAGGTAACCGGCCTCTGTTCGAGG
ACATGACCGACTCCGATTGCCGGGACAATGCCCCCCGGACCATCTTC
ATTATCTCCATGTACAAGGACAGCCAGCCCCGGGGCATGGCTGTGAC
AATTAGCGTGAAGTGTGAGAAAATCAGCACTTTATCTTGTGAGAACA
AGATCATCTCCTTTAAGGAAATGAACCCCCCCGATAACATCAAGGAC
ACCAAGTCCGATATCATCTTCTTCCAGCGGTCCGTGCCCGGTCACGA
TAACAAGATGCAGTTCGAATCCTCCTCCTACGAGGGCTACTTTTTAG
CTTGTGAAAAGGAGAGGGATTTATTCAAGCTGATCCTCAAGAAGGAG
GACGAGCTGGGCGATCGTTCCATCATGTTCACCGTCCAAAACGAGGA
T
Human Soluble PDGF-DD
(SEQ ID NO: 26)
RDTSATPQSASIKALRNANLRRDESNHLTDLYRRDETIQVKGNGYVQ
SPRFPNSYPRNLLLTWRLHSQENTRIQLVFDNQFGLEEAENDICRYD
FVEVEDISETSTIIRGRWCGHKEVPPRIKSRTNQIKITFKSDDYFVA
KPGFKIYYSLLEDFQPAAASETNWESVTSSISGVSYNSPSVTDPTLI
ADALDKKIAEFDTVEDLLKYFNPESWQEDLENMYLDTPRYRGRSYHD
RKSKVDLDRLNDDAKRYSCTPRNYSVNIREELKLANVVFFPRCLLVQ
RCGGNCGCGTVNWRSCTCNSGKTVKKYHEVLQFEPGHIKRRGRAKTM
ALVDIQLDHHERCDCICSSRPPR
Human Soluble SCF
(SEQ ID NO: 27)
EGICRNRVTNNVKDVTKLVANLPKDYMITLKYVPGMDVLPSHCWISE
MVVQLSDSLTDLLDKFSNISEGLSNYSIIDKLVNIVDDLVECVKENS
SKDLKKSFKSPEPRLFTPEEFFRIFNRSIDAFKDFVVASETSDCVVS
STLSPEKDSRVSVTKPFMLPPVAASSLRNDSSSSNRKAKNPPGDSSL
HWAAMALPALFSLIIGFAFGALYWKKRQPSLTRAVENIQINEEDNEI
SMLQEKEREFQEV
Human Soluble FLT3L
(SEQ ID NO: 28)
TQDCSFQHSPISSDFAVKIRELSDYLLQDYPVTVASNLQDEELCGGL
WRLVLAQRWMERLKTVAGSKMQGLLERVNTEIHFVTKCAFQPPPSCL
RFVQTNISRLLQETSEQLVALKPWITRQNFSRCLELQCQPDSSTLPP
PWSPRPLEATAPTAPQPPLLLLLLLPVGLLLLAAAWCLHWQRTRRRT
PRPGEQVPPVPSPQDLLLVEH
Mouse IL-4
(SEQ ID NO: 90)
HIHGCDKNHLREIIGILNEVTGEGTPCTEMDVPNVLTATKNTTESEL
VCRASKVLRIFYLKHGKTPCLKKNSSVLMELQRLFRAFRCLDSSISC
TMNESKSTSLKDFLESLKSIMQMDYS
Human IL-4
(SEQ ID NO: 91)
MGLTSQLLPPLFFLLACAGNFVHGHKCDITLQEIIKTLNSLTEQKTL
CTELTVTDIFAASKNTTEKETFCRAATVLRQFYSHHEKDTRCLGATA
QQFHRHKQLIRFLKRLDRNLWGLAGLNSCPVKEANQSTLENFLERLK
TIMREKYSKCSS

Exemplary soluble cell surface proteins include soluble MICA, MICB, and a ULP16 binding protein (e.g., ULBP1, ULBP2, ULBP3, ULBP4, ULBP5, or ULBP6). Exemplary sequences for soluble MICA, MICB, ULBP1, ULBP2, ULBP3, ULBP4, ULBP5, and ULBP6 are listed below.

Human Soluble MICA
(SEQ ID NO: 29)
EPHSLRYNLTVLSWDGSVQSGFLTEVHLDGQPFLRCDROKCRAKPQGQWAEDVL
GNKTWDRETRDLTGNGKDLRMTLAHIKDQKEGLHSLQEIRVCEIHEDNSTRSSQ
HFYYDGELFLSQNLETKEWTMPQSSRAQTLAMNVRNFLKEDAMKTKTHYHAMHA
DCLQELRRYLKSGVVLRRTVPPMVNVTRSEASEGNITVTCRASGFYPWNITLSW
RQDGVSLSHDTQQWGDVLPDGNGTYQTWVATRICQGEEQRFTCYMEHSGNHSTH
PVPSGKVLVLQSHWQTFHVSAVAAAAIFVIIIFYVRCCKKKTSAAEGPELVSLQ
VLDQHPVGTSDHRDATQLGFQPLMSDLGSTGSTEGA
Human Soluble MICB
(SEQ ID NO: 30)
AEPHSLRYNLMVLSQDESVQSGFLAEGHLDGQPFLRYDRQKRRAKPQGQWAEDV
LGAKTWDTETEDLTENGQDLRRTLTHIKDQKGGLHSLQEIRVCEIHEDSSTRGS
RHFYYDGELFLSQNLETQESTVPQSSRAQTLAMNVTNFWKEDAMKTKTHYRAMQ
ADCLQKLQRYLKSGVAIRRTVPPMVNVTCSEVSEGNITVTCRASSFYPRNITLT
WRQDGVSLSHNTQQWGDVLPDGNGTYQTWVATRIRQGEEQRFTCYMEHSGNHGT
HPVPSGKVLVLQSQRTDFPYVSAAMPCFVIIIILCVPCCKKKTSAAEGPELVSL
QVLDQHPVGTGDHRDAAQLGFQPLMSATGSTGSTEGA
Human Soluble ULBP1
(SEQ ID NO: 31)
WVDTHCLCYDFIITPKSRPEPQWCEVQGLVDERPFLHYDCVNHKAKAFASLGKK
VNVTKTWEEQTETLRDVVDFLKGQLLDIQVENLIPIEPLTLQARMSCEHEAHGH
GRGSWQFLFNGQKFLLFDSNNRKWTALHPGAKKMTEKWEKNRDVTMFFQKISLG
DCKMWLEEFLMYWEQMLDPTKPPSLAPG
Human Soluble ULBP2
(SEQ ID NO: 32)
GRADPHSLCYDITVIPKFRPGPRWCAVQGQVDEKTFLHYDCGNKTVTPVSPLGK
KLNVTTAWKAQNPVLREVVDILTEQLRDIQLENYTPKEPLTLQARMSCEQKAEG
HSSGSWQFSFDGQIFLLFDSEKRMWTTVHPGARKMKEKWENDKVVAMSFHYFSM
GDCIGWLEDFLMGMDSTLEPSAGAPLAMS
Human Soluble ULBP3
(SEQ ID NO: 33)
DAHSLWYNFTIIHLPRHGQQWCEVOSQVDQKNFLSYDCGSDKVLSMGHLEEQLY
ATDAWGKOLEMLREVGORLRLELADTELEDFTPSGPLTLQVRMSCECEADGYIR
GSWQFSFDGRKFLLFDSNNRKWTVVHAGARRMKEKWEKDSGLTTFFKMVSMRDC
KSWLRDFLMHRKKRLEPTAPPTMAPG
Human Soluble ULBP4
(SEQ ID NO: 34)
HSLCFNFTIKSLSRPGQPWCEAQVFLNKNLFLQYNSDNNMVKPLGLLGKKVYAT
STWGELTQTLGEVGRDLRMLLCDIKPQIKTSDPSTLQVEMFCQREAERCTGASW
QFATNGEKSLLFDAMNMTWTVINHEASKIKETWKKDRGLEKYFRKLSKGDCDHW
LREFLGHWEAMPEPTVSPVNASDIHWSSSSLPDRWIILGAFILLVLMGIVLICV
WWQNGEWQAGLWPLRTS
Human Soluble ULBP5
(SEQ ID NO: 35)
GLADPHSLCYDITVIPKFRPGPRWCAVQGQVDEKTFLHYDCGSKTVTPVSPLGK
KLNVTTAWKAQNPVLREVVDILTEQLLDIQLENYIPKEPLTLQARMSCEQKAEG
HGSGSWQLSFDGQIFLLFDSENRMWTTVHPGARKMKEKWENDKDMTMSFHYISM
GDCTGWLEDFLMGMDSTLEPSAGAPPTMSSG
Human Soluble ULBP6
(SEQ ID NO: 36)
RRDDPHSLCYDITVIPKFRPGPRWCAVQGQVDEKTFLHYDCGNKTVTPVSPLGK
KLNVTMAWKAQNPVLREVVDILTEQLLDIQLENYTPKEPLTLQARMSCEQKAEG
HSSGSWQFSIDGQTFLLFDSEKRMWTTVHPGARKMKEKWENDKDVAMSFHYISM
GDCIGWLEDFLMGMDSTLEPSAGAPLAMSSG

In some embodiments, a soluble IL-12 protein can include a first sequence that is at least 70% identical (e.g., at least 75% identical, at least 80% identical, at least 85% identical, at least 90% identical, at least 95% identical, at least 99% identical, or 100% identical) to SEQ ID NO: 16, and a second sequence that is at least 70% identical (e.g., at least 75% identical, at least 80% identical, at least 85% identical, at least 90% identical, at least 95% identical, at least 99% identical, or 100% identical) to SEQ ID NO: 18. In some embodiments, the soluble IL-12 can further include a linker sequence (e.g., any of the exemplary linker sequences described herein or known in the art) between the first sequence and the second sequence.

In some embodiments, a soluble IL-12 protein is encoded by a first nucleic acid encoding a first sequence at least 70% identical (e.g., at least 75% identical, at least 80% identical, at least 85% identical, at least 90% identical, at least 95% identical, at least 99% identical, or 100% identical) to SEQ ID NO: 17, and a second nucleic acid sequence encoding a second sequence that is at least 70% identical (e.g., at least 75% identical, at least 80% identical, at least 85% identical, at least 90% identical, at least 95% identical, at least 99% identical, or 100% identical) to SEQ ID NO: 19. In some embodiments, the nucleic acid encoding a soluble IL-12 protein further includes a nucleic acid sequence encoding a linker sequence (e.g., any of the exemplary linker sequences described herein or known in the art) between the first nucleic acid and the second nucleic acid.

In some embodiments, a soluble IL-12 protein includes a sequence that is at least 70% identical (e.g., at least 75% identical, at least 80% identical, at least 85% identical, at least 90% identical, at least 95% identical, at least 99% identical, or 100% identical) to SEQ ID NO: 20. In some embodiments, a soluble IL-12 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 21.

In some embodiments, a soluble IL-18 protein can include a sequence that is at least 70% identical (e.g., at least 75% identical, at least 80% identical, at least 85% identical, at least 90% identical, at least 95% identical, at least 99% identical, or 100% identical) to SEQ ID NO: 24. In some embodiments, a soluble IL-18 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 25.

Additional examples of soluble interleukin proteins and soluble cytokine proteins are known in the art.

### Soluble Interleukin or Cytokine Receptor

In some embodiments of any of the single-chain chimeric polypeptides described herein, one or both of the first target-binding domain and the second target-binding domain is a soluble interleukin receptor, a soluble cytokine receptor, or a soluble cell surface receptor. In some embodiments, the soluble receptor is a soluble TGF-β receptor II (TGF-βRII) (see, e.g., those described in Yung et al., Am. J. Resp. Crit. Care Med. 194 (9): 1140-1151, 2016), a soluble TGF-βRIII (see, e.g., those described in Heng et al., Placenta 57:320, 2017), or a soluble NKG2D (see, e.g., Cosman et al., Immunity 14 (2): 123-133, 2001; Costa et al., Front. Immunol., Vol. 9, Article 1150 May 29, 2018; doi: 10.3389/fimmu.2018.01150). In some embodiments, the soluble cell surface receptor is a soluble NKp30 (see, e.g., Costa et al., Front. Immunol., Vol. 9, Article 1150 May 29, 2018; doi: 10.3389/fimmu.2018.01150), a soluble NKp44 (see, e.g., those described in Costa et al., Front. Immunol., Vol. 9, Article 1150 May 29, 2018; doi: 10.3389/fimmu.2018.01150), a soluble NKp46 (see, e.g., Mandelboim et al., Nature 409:1055-1060, 2001; Costa et al., Front. Immunol., Vol. 9, Article 1150 May 29, 2018; doi: 10.3389/fimmu.2018.01150), a soluble DNAM1 (see, e.g., those described in Costa et al., Front. Immunol., Vol. 9, Article 1150 May 29, 2018; doi: 10.3389/fimmu.2018.01150), a scMHCI (see, e.g., those described in Washburn et al., PLoS One 6 (3): e18439, 2011), a scMHCII (see, e.g., those described in Bishwajit et al., Cellular Immunol. 170 (1): 25-33, 1996), a scTCR (see, e.g., those described in Weber et al., Nature 356 (6372): 793-796, 1992), a soluble CD155 (see, e.g., those described in Tahara-Hanaoka et al., Int. Immunol. 16 (4): 533-538, 2004), or a soluble CD28 (see, e.g., Hebbar et al., Clin. Exp. Immunol. 136:388-392, 2004).

### Exemplary Soluble TGFβRII Receptor

In some embodiments, a target-binding domain can comprise a soluble TGFβRII receptor. In some embodiments, a soluble TGFβRII receptor can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 7)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 7)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments, the soluble TGFβRII receptor can further include a linker sequence (e.g., any of the exemplary linker sequences described herein or known in the art) between the first sequence and the second sequence.

In some embodiments, a soluble TGFβRII receptor is encoded by a first nucleic acid encoding a first sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 8)
ATCCCGCCACACGTGCAAAAGAGCGTGAACAACGACATGATCGTTACGG
ACAACAATGGGGCCGTGAAGTTCCCTCAGTTATGCAAATTCTGTGACGT
GAGATTCAGCACGTGCGACAATCAGAAGAGCTGCATGTCCAATTGCTCA
ATCACAAGCATCTGCGAGAAACCCCAAGAAGTTTGCGTGGCCGTGTGGA
GAAAGAATGACGAGAACATCACCCTGGAAACCGTGTGCCATGATCCTAA
ACTCCCTTACCATGACTTTATCTTGGAGGATGCTGCTAGCCCAAAGTGC
ATCATGAAAGAGAAGAAGAAACCGGGGGAAACTTTTTTCATGTGTTCAT
GCAGCTCTGACGAGTGTAATGATAACATTATCTTCAGCGAAGAGTATAA
CACTAGCAATCCTGAT.

or and a second nucleic acid sequence encoding a second sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 37)
ATCCCCCCCCATGTGCAAAAATCAGTGAACAATGACATGATCGTGACCG
ATAATAACGGGGCGGTAAAGTTTCCTCAACTGTGTAAATTTTGCGACGT
CAGATTCAGCACATGCGACAATCAAAAGTCCTGCATGAGCAACTGCAGC
ATCACCTCTATCTGTGAGAAACCACAAGAAGTATGTGTGGCGGTATGGC
GGAAGAACGATGAAAATATCACGCTGGAGACCGTTTGCCATGATCCTAA
GCTGCCATATCACGATTTCATCCTGGAGGACGCCGCTAGCCCTAAGTGT
ATCATGAAGGAGAAGAAGAAGCCCGGTGAGACCTTTTTCATGTGCTCAT
GCAGCTCCGACGAGTGCAACGACAACATCATCTTCAGCGAGGAGTACAA
TACAAGCAATCCTGAC.

In some embodiments, the nucleic acid encoding a soluble TGFβRII receptor further includes a nucleic acid sequence encoding a linker sequence (e.g., any of the exemplary linker sequences described herein or known in the art) between the first nucleic acid and the second nucleic acid.

In some embodiments, a soluble TGFβRII receptor is encoded by a first nucleic acid encoding a first sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 38)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCACG
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGAT,

and a second nucleic acid sequence encoding a second sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 38)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCACG
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGAT.

In some embodiments, the nucleic acid encoding a soluble TGFβRII receptor further includes a nucleic acid sequence encoding a linker sequence (e.g., any of the exemplary linker sequences described herein or known in the art) between the first nucleic acid and the second nucleic acid.

In some embodiments, a soluble TGFβRII receptor includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 9)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPD,
or
(SEQ ID NO: 39)
IPPHVQKSVNNDMIVTDNNGAVKFPQLCKFCDVRFSTCDNOKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD.

In some embodiments, a soluble TGFβRII receptor is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 10)
ATCCCGCCACACGTGCAAAAGAGCGTGAACAACGACATGATCGTTACGGACAAC
AATGGGGCCGTGAAGTTCCCTCAGTTATGCAAATTCTGTGACGTGAGATTCAGC
ACGTGCGACAATCAGAAGAGCTGCATGTCCAATTGCTCAATCACAAGCATCTGC
GAGAAACCCCAAGAAGTTTGCGTGGCCGTGTGGAGAAAGAATGACGAGAACATC
ACCCTGGAAACCGTGTGCCATGATCCTAAACTCCCTTACCATGACTTTATCTTG
GAGGATGCTGCTAGCCCAAAGTGCATCATGAAAGAGAAGAAGAAACCGGGGGAA
ACTTTTTTCATGTGTTCATGCAGCTCTGACGAGTGTAATGATAACATTATCTTC
AGCGAAGAGTATAACACTAGCAATCCTGATGGAGGTGGCGGATCTATCCCCCCC
CATGTGCAAAAATCAGTGAACAATGACATGATCGTGACCGATAATAACGGGGCG
GTAAAGTTTCCTCAACTGTGTAAATTTTGCGACGTCAGATTCAGCACATGCGAC
AATCAAAAGTCCTGCATGAGCAACTGCAGCATCACCTCTATCTGTGAGAAACCA
CAAGAAGTATGTGTGGCGGTATGGCGGAAGAACGATGAAAATATCACGCTGGAG
ACCGTTTGCCATGATCCTAAGCTGCCATATCACGATTTCATCCTGGAGGACGCC
GCTAGCCCTAAGTGTATCATGAAGGAGAAGAAGAAGCCCGGTGAGACCTTTTTC
ATGTGCTCATGCAGCTCCGACGAGTGCAACGACAACATCATCTTCAGCGAGGAG
TACAATACAAGCAATCCTGAC,
or
(SEQ ID NO: 40)
ATCCCCCCCCATGTGCAAAAGAGCGTGAACAACGATATGATCGTGACCGACAAC
AACGGCGCCGTGAAGTTTCCCCAGCTCTGCAAGTTCTGCGATGTCAGGTTCAGC
ACCTGCGATAATCAGAAGTCCTGCATGTCCAACTGCACGATCACCTCCATCTGC
GAGAAGCCCCAAGAAGTGTGCGTGGCCGTGTGGCGGAAAAATGACGAGAACATC
ACCCTGGAGACCGTGTGTCACGACCCCAAGCTCCCTTATCACGACTTCATTCTG
GAGGACGCTGCCTCCCCCAAATGCATCATGAAGGAGAAGAAGAAGCCCGGAGAG
ACCTTCTTTATGTGTTCCTGTAGCAGCGACGAGTGTAACGACAACATCATCTTC
AGCGAAGAGTACAACACCAGCAACCCTGATGGAGGTGGCGGATCCGGAGGTGGA
GGTTCTGGTGGAGGTGGGAGTATTCCTCCCCACGTGCAGAAGAGCGTGAATAAT
GACATGATCGTGACCGATAACAATGGCGCCGTGAAATTTCCCCAGCTGTGCAAA
TTCTGCGATGTGAGGTTTTCCACCTGCGACAACCAGAAGTCCTGTATGAGCAAC
TGCACAATCACCTCCATCTGTGAGAAGCCTCAGGAGGTGTGCGTGGCTGTCTGG
CGGAAGAATGACGAGAATATCACCCTGGAAACCGTCTGCCACGATCCCAAGCTG
CCCTACCACGATTTCATCCTGGAAGACGCCGCCAGCCCTAAGTGCATCATGAAA
GAGAAAAAGAAGCCTGGCGAGACCTTTTTCATGTGCTCCTGCAGCAGCGACGAA
TGCAACGACAATATCATCTTTAGCGAGGAATACAATACCAGCAACCCCGAC,
or
(SEQ ID NO: 92)
ATTCCTCCTCACGTGCAGAAAAGCGTGAACAACGACATGATAGTGACCGACAAT
AACGGCGCGGTCAAGTTCCCACAGCTCTGCAAATTTTGCGACGTGAGATTTAGT
ACCTGCGACAATCAGAAATCCTGCATGAGCAATTGTAGCATCACTAGCATCTGT
GAAAAGCCCCAAGAGGTCTGCGTAGCAGTGTGGAGAAAAAATGACGAGAACATC
ACACTAGAGACCGTGTGCCATGACCCTAAGCTGCCTTATCATGATTTCATCCTT
GAAGATGCTGCTAGCCCTAAGTGCATTATGAAAGAGAAAAAGAAGCCTGGCGAA
ACCTTTTTCATGTGTAGCTGTAGTAGCGACGAGTGCAACGATAACATCATCTTC
TCGGAGGAGTATAATACATCGAATCCTGATGGCGGCGGCGGCAGCATCCCTCCT
CATGTTCAGAAGAGCGTGAATAACGACATGATTGTGACGGATAACAATGGCGCC
GTAAAGTTCCCTCAGCTGTGTAAGTTCTGCGATGTGCGATTTAGCACCTGCGAC
AATCAGAAGAGCTGTATGTCCAATTGTTCAATCACAAGCATCTGTGAGAAGCCT
CAAGAGGTGTGCGTGGCCGTTTGGAGAAAGAACGACGAGAACATTACCCTTGAG
ACCGTATGCCACGATCCTAAGCTGCCGTATCACGACTTCATACTGGAGGATGCC
GCTAGCCCTAAATGCATCATGAAAGAAAAAAAAAAGCCTGGTGAGACATTTTTT
ATGTGTAGCTGCTCGTCTGACGAGTGCAATGACAATATCATTTTCAGCGAAGAA
TATAACACCTCAAACCCTGAC.

Additional examples of soluble interleukin receptors and soluble cytokine receptors are known in the art.

### Antigen-Binding Domains

In some embodiments of any of the single-chain chimeric polypeptides described herein, one or both of the first target-binding domain and the second target-binding domain is an antigen-binding domain. In some embodiments of any of the single-chain chimeric polypeptides described herein, the first target-binding domain and the second target-binding domain are each an antigen-binding domain.

In some embodiments of any of the single-chain chimeric polypeptides described herein, the first target-binding domain and the second target-binding domain bind specifically to the same antigen. In some embodiments of these single-chain chimeric polypeptides, the first target-binding domain and the second target-binding domain bind specifically to the same epitope. In some embodiments of these single-chain chimeric polypeptides, the first target-binding domain and the second target-binding domain include the same amino acid sequence.

In some embodiments of any of the single-chain chimeric polypeptides described herein, the first target-binding domain and the second target-binding domain bind specifically to different antigens.

In some embodiments of any of the antigen-binding domains described herein can bind to an antigen selected from the group consisting of: a protein, a carbohydrate, a lipid, and a combination thereof.

In some examples, an antigen-binding domain (e.g., any of the antigen-binding domains described herein) can bind specifically to any one of tissue factor (CD142) (see e.g., those described in U.S. Pat. Nos. 9,714,297; 8,951,525; 11,447,566; and 9,708,410), CD16a (see, e.g., those described in U.S. Pat. No. 9,035,026), CD28 (see, e.g., those described in U.S. Pat. No. 7,723,482), CD3 (see, e.g., those described in U.S. Pat. No. 9,226,962), CD33 (see, e.g., those described in U.S. Pat. No. 8,759,494), CD20 (see, e.g., those described in WO 2014/026054), CD19 (see, e.g., those described in U.S. Pat. No. 9,701,758), CD22 (see, e.g., those described in WO 2003/104425), CD123 (see, e.g., those described in WO 2014/130635), IL-1R (see, e.g., those described in U.S. Pat. No. 8,741,604), IL-1 (see, e.g., those described in WO 2014/095808), VEGF (see, e.g., those described in U.S. Pat. No. 9,090,684), IL-6R (see, e.g., those described in U.S. Pat. No. 7,482,436), IL-4 (see, e.g., those described in U.S. Patent Application Publication No. 2012/0171197), IL-10 (see, e.g., those described in U.S. Patent Application Publication No. 2016/0340413), PDL-1 (see, e.g., those described in Drees et al., Protein Express. Purif. 94:60-66, 2014), TIGIT (see, e.g., those described in U.S. Patent Application Publication No. 2017/0198042), PD-1 (see, e.g., those described in U.S. Pat. No. 7,488,802), TIM3 (see, e.g., those described in U.S. Pat. No. 8,552,156), CTLA4 (see, e.g., those described in WO 2012/120125), ICOS (see, e.g., those described in Humbin et al., Immunity 58:1966-1983, 2025), MICA (see, e.g., those described in WO 2016/154585), MICB (see, e.g., those described in U.S. Pat. No. 8,753,640), IL-6 (see, e.g., those described in Gejima et al., Human Antibodies 11 (4): 121-129, 2002), IL-8 (see, e.g., those described in U.S. Pat. No. 6,117,980), TNFα (see, e.g., those described in Geng et al., Immunol. Res. 62 (3): 377-385, 2015), CD26a (see, e.g., those described in WO 2017/189526), CD36 (see, e.g., those described in U.S. Patent Application Publication No. 2015/0259429), ULBP2 (see, e.g., those described in U.S. Pat. No. 9,273,136), CD30 (see, e.g., those described in Homach et al., Scand. J. Immunol. 48 (5): 497-501, 1998), CD200 (see, e.g., those described in U.S. Pat. No. 9,085,623), IGF-1R (see, e.g., those described in U.S. Patent Application Publication No. 2017/0051063), MUC4AC (see, e.g., those described in WO 2012/170470), MUC5AC (see, e.g., those described in U.S. Pat. No. 9,238,084), Trop-2 (see, e.g., those described in WO 2013/068946), CMET (see, e.g., those described in Edwardraja et al., Biotechnol. Bioeng. 106 (3): 367-375, 2010), EGFR (see, e.g., those described in Akbari et al., Protein Expr. Purif. 127:8-15, 2016), HER1 (see, e.g., those described in U.S. Patent Application Publication No. 2013/0274446), HER2 (see, e.g., those described in Cao et al., Biotechnol. Lett. 37 (7): 1347-1354, 2015), HER3 (see, e.g., those described in U.S. Pat. No. 9,505,843), PSMA (see, e.g., those described in Parker et al., Protein Expr. Purif. 89 (2): 136-145, 2013), CEA (see, e.g., those described in WO 1995/015341), B7H3 (see, e.g., those described in U.S. Pat. No. 9,371,395), EPCAM (see, e.g., those described in WO 2014/159531), BCMA (see, e.g., those described in Smith et al., Mol. Ther. 26 (6): 1447-1456, 2018), P-cadherin (see, e.g., those described in U.S. Pat. No. 7,452,537), CEACAM5 (see, e.g., those described in U.S. Pat. No. 9,617,345), a UL16-binding protein (see, e.g., those described in WO 2017/083612), HLA-DR (see, e.g., Pistillo et al., Exp. Clin. Immunogenet. 14 (2): 123-130, 1997), DLL4 (see, e.g., those described in WO 2014/007513), TYRO3 (see, e.g., those described in WO 2016/166348), AXL (see, e.g., those described in WO 2012/175692), MER (see, e.g., those described in WO 2016/106221), CD122 (see, e.g., those described in U.S. Patent Application Publication No. 2016/0367664), CD155 (see, e.g., those described in WO 2017/149538), or PDGFDD (see, e.g., those described in U.S. Pat. No. 9,441,034).

In some embodiments of any of the single-chain chimeric polypeptides described herein, the antigen-binding domain includes or is a scFv or a single domain antibody (e.g., a V_{H}H or a V_{NAR} domain). In some embodiments, the antigen-binding domains present in any of the single-chain chimeric polypeptides described herein are each independently selected from the group consisting of: a VHH domain, a VNAR domain, and a scFv.

A VHH domain is a single monomeric variable antibody domain that can be found in camelids. A V_{NAR} domain is a single monomeric variable antibody domain that can be found in cartilaginous fish. Non-limiting aspects of VHH domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, each of the antigen-binding domains in the single-chain chimeric polypeptides described herein are both VHH domains, or at least one antigen-binding domain is a VHH domain. In some embodiments, each of the antigen-binding domains in the single-chain chimeric polypeptides described herein are both VNAR domains, or at least one antigen-binding domain is a VNAR domain. In some embodiments, each of the antigen-binding domains in the single-chain chimeric polypeptides described herein are both scFv domains, or at least one antigen-binding domain is a scFv domain.

In some embodiments, any of the antigen-binding domains described herein is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. DARTs are described in, e.g., Garber, Nature Reviews Drug Discovery 13:799-801, 2014. Additional examples of antigen-binding domains that can be used in any of the single-chain chimeric polypeptide are known in the art.

In some embodiments, any of the antigen-binding domains described herein is an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a 1 mmTAC, an IgG-IgG conjugate, a Cov-X-Body, and a scFv1-PEG-scFv2. See, e.g., Spiess et al., Mol. Immunol. 67:95-106, 2015, incorporated in its entirety herewith, for a description of these elements. In some embodiments, two or more polypeptides present in the single-chain chimeric polypeptide assemble (e.g., non-covalently assemble) to form any of the antigen-binding domains described herein. In some embodiments, the two or more polypeptides present in the single-chain chimeric polypeptide are connected with a peptide linker sequence. For example, two or more polypeptide chains connected by a linker can assemble (e.g., non-covalently assemble) to form any of the antigen-binding domains described herein.

Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

Exemplary antigen-binding domains are provided below.

### Anti-CD3 Antigen-Binding Domains

In some embodiments of any of the single-chain chimeric polypeptides described herein, one or more of the first target-binding domain, the second target-binding domain, and one or more of the one or more additional antigen-binding domains can be an anti-CD3 antigen-binding domain.

In some embodiments, the first target-binding domain, the second target-binding domain, and/or one or more of the one or more additional antigen-binding domains can be an anti-CD3 scFv.

In some embodiments, an anti-CD3 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 93)
DIQMTQSPSSLSASVGDRVTITCRASQDIRNYLNWYQQKPGKAPKLLIY
YTSRLESGVPSRFSGSGSGTDYTLTISSLQPEDFATYYCQQGNTLPWTF
GQGTKVEIKGGGGSGGGGSGGGGSEVQLVESGGGLVQPGGSLRLSCAAS
GYSFTGYTMNWVRQAPGKGLEWVALINPYKGVSTYNQKFKDRFTISVDK
SKNTAYLOMNSLRAEDTAVYYCARSGYYGDSDWYFDVWGQGTLVTVSS.

In some embodiments, an anti-CD3 scFv is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 94)
GATATTCAGATGACGCAGTCGCCAAGCTCACTCTCGGCCTCCGTAGGAG
ACAGAGTTACCATCACCTGCAGAGCTAGCCAAGACATCAGAAACTACCT
GAACTGGTATCAGCAGAAGCCTGGCAAGGCCCCTAAGCTCCTGATCTAC
TACACGAGCAGACTTGAGAGCGGGGTACCATCCCGCTTCAGCGGATCTG
GAAGCGGCACAGACTACACCCTGACCATCTCTAGTCTGCAGCCTGAGGA
CTTCGCCACTTACTATTGTCAGCAAGGCAACACCCTGCCTTGGACCTTT
GGCCAAGGCACCAAAGTTGAGATAAAGGGAGGGGGAGGGAGCGGTGGCG
GAGGCTCGGGTGGGGGTGGCTCAGAAGTACAGCTGGTTGAGTCCGGCGG
TGGTCTGGTGCAGCCTGGCGGAAGCCTGAGACTGAGCTGCGCCGCAAGC
GGCTATAGCTTCACTGGCTATACCATGAACTGGGTGCGCCAAGCCCCTG
GCAAGGGCCTGGAATGGGTGGCCCTGATCAACCCTTACAAGGGCGTGAG
CACCTACAATCAGAAGTTCAAGGACAGATTCACCATCAGCGTGGACAAG
AGCAAGAACACCGCCTACCTGCAGATGAACAGCCTGAGAGCCGAGGACA
CTGCGGTCTATTACTGTGCTAGAAGCGGCTACTACGGCGACAGCGACTG
GTACTTCGACGTGTGGGGGCAAGGCACCTTGGTAACGGTAAGCTCC.

In some embodiments, an anti-CD3 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 95)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSVEGGSGGSGGSGGSGGVDDIQLTQSPAI
MSASPGEKVTMTCRASSSVSYMNWYQQKSGTSPKRWIYDTSKVASGVPY
RFSGSGSGTSYSLTISSMEAEDAATYYCQQWSSNPLTFGAGTKLELK.

In some embodiments, an anti-CD3 scFv is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 96)
GACATCAAGCTGCAGCAGAGCGGCGCCGAGCTGGCTAGACCTGGGGCTA
GCGTCAAGATGAGCTGCAAGACAAGCGGCTACACTTTCACAAGATACAC
CATGCACTGGGTGAAGCAGAGACCTGGCCAAGGCCTGGAATGGATCGGT
TATATCAACCCTAGCAGAGGCTACACCAACTACAATCAGAAGTTCAAGG
ACAAAGCCACCCTGACAACTGATAAGAGCAGCAGCACCGCCTATATGCA
ACTGTCAAGCCTCACCTCAGAGGACAGCGCCGTGTACTACTGCGCTAGA
TACTACGACGACCACTACTGCCTGGACTATTGGGGCCAAGGCACTACTT
TGACTGTTAGCTCGGTCGAGGGAGGCTCCGGCGGGAGTGGTGGTTCTGG
CGGCAGCGGCGGAGTTGACGACATTCAGCTGACACAAAGTCCTGCCATA
ATGAGCGCTAGCCCTGGCGAGAAGGTGACCATGACCTGCAGAGCTAGCA
GCAGCGTGAGCTACATGAACTGGTATCAGCAGAAGAGCGGCACAAGCCC
TAAAAGATGGATCTACGACACAAGCAAGGTGGCTAGCGGCGTGCCTTAC
CGGTTTAGCGGCTCTGGTAGCGGCACAAGCTATAGCCTCACGATTAGCT
CAATGGAGGCCGAGGACGCCGCCACGTATTATTGCCAACAGTGGAGCAG
CAACCCTCTGACCTTCGGCGCCGGGACCAAGCTGGAACTTAAG.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to

(SEQ ID NO: 97)
DIQMTQSPSSLPASLGDRVTINCQASQDISNYLNWYQQKPGKAPKLLIY
YTNKLADGVPSRFSGSGSGRDSSFTISSLESEDIGSYYCQQYYNYPWTF
GPGTKLEIKGGGGSGGGGSGGGGSEVQLVESGGGLVQPGKSLKLSCEAS
GFTFSGYGMHWVRQAPGRGLESVAYITSSSINIKYADAVKGRFTVSRDN
AKNLLFLQMNILKSEDTAMYYCARFDWDKNYWGQGTMVTVSS.

In some embodiments, the anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to:

(SEQ ID NO: 41)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSS.

and/or a light chain variable domain including a sequence that is at least 70% identical (e.g., at least 75% identical, at least 80% identical, at least 85% identical, at least 90% identical, at least 95% identical, at least 99% identical, or 100% identical) to:

(SEQ ID NO: 42)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINR.

In some embodiments, a scFv (e.g., any of the scFvs described herein) can include a linker sequence (e.g., any of the exemplary linker sequences described herein or known in the art) between the heavy chain variable domain and the light chain variable domain. In some embodiments, the anti-CD3 scFv can include a heavy chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to:

(SEQ ID NO: 43)
CAAGTTCAGCTCCAGCAAAGCGGCGCCGAACTCGCTCGGCCCGGCGCTTC
CGTGAAGATGTCTTGTAAGGCCTCCGGCTATACCTTCACCCGGTACACAA
TGCACTGGGTCAAGCAACGGCCCGGTCAAGGTTTAGAGTGGATTGGCTAT
ATCAACCCCTCCCGGGGCTATACCAACTACAACCAGAAGTTCAAGGACAA
AGCCACCCTCACCACCGACAAGTCCAGCAGCACCGCTTACATGCAGCTGA
GCTCTTTAACATCCGAGGATTCCGCCGTGTACTACTGCGCTCGGTACTAC
GACGATCATTACTGCCTCGATTACTGGGGCCAAGGTACCACCTTAACAGT
CTCCTCC,

and/or a light chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to:

(SEQ ID NO: 44)
CAGATCGTGCTGACCCAGTCCCCCGCTATTATGAGCGCTAGCCCCGGTGA
AAAGGTGACTATGACATGCAGCGCCAGCTCTTCCGTGAGCTACATGAACT
GGTATCAGCAGAAGTCCGGCACCAGCCCTAAAAGGTGGATCTACGACACC
AGCAAGCTGGCCAGCGGCGTCCCCGCTCACTTTCGGGGCTCCGGCTCCGG
AACAAGCTACTCTCTGACCATCAGCGGCATGGAAGCCGAGGATGCCGCTA
CCTATTACTGTCAGCAGTGGAGCTCCAACCCCTTCACCTTTGGATCCGGC
ACCAAGCTCGAGATTAATCGT.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to:

(SEQ ID NO: 45)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRGGGGSGGGGSGGGGSQVQLQQSGAELARPGASVKMSCKASGYT
FTRYTMHWVKQRPGQGLEWIGYINPSRGYTNYNQKFKDKATLTTDKSSST
AYMQLSSLTSEDSAVYYCARYYDDHYCLDYWGQGTTLTVSS.

In some embodiments, an anti-CD3 scFv can include the six CDRs present in SEQ ID NO: 45.

In some embodiments, an anti-CD3 scFv can include a sequence encoded by a nucleic acid sequence that is at least 70% identical (e.g., at least 75% identical, at least 80% identical, at least 85% identical, at least 90% identical, at least 95% identical, at least 99% identical, or 100% identical) to:

(SEQ ID NO: 46)
CAGATCGTGCTGACCCAGTCCCCCGCTATTATGAGCGCTAGCCCCGGTGA
AAAGGTGACTATGACATGCAGCGCCAGCTCTTCCGTGAGCTACATGAACT
GGTATCAGCAGAAGTCCGGCACCAGCCCTAAAAGGTGGATCTACGACACC
AGCAAGCTGGCCAGCGGCGTCCCCGCTCACTTTCGGGGCTCCGGCTCCGG
AACAAGCTACTCTCTGACCATCAGCGGCATGGAAGCCGAGGATGCCGCTA
CCTATTACTGTCAGCAGTGGAGCTCCAACCCCTTCACCTTTGGATCCGGC
ACCAAGCTCGAGATTAATCGTGGAGGCGGAGGTAGCGGAGGAGGCGGATC
CGGCGGTGGAGGTAGCCAAGTTCAGCTCCAGCAAAGCGGCGCCGAACTCG
CTCGGCCCGGCGCTTCCGTGAAGATGTCTTGTAAGGCCTCCGGCTATACC
TTCACCCGGTACACAATGCACTGGGTCAAGCAACGGCCCGGTCAAGGTTT
AGAGTGGATTGGCTATATCAACCCCTCCCGGGGCTATACCAACTACAACC
AGAAGTTCAAGGACAAAGCCACCCTCACCACCGACAAGTCCAGCAGCACC
GCTTACATGCAGCTGAGCTCTTTAACATCCGAGGATTCCGCCGTGTACTA
CTGCGCTCGGTACTACGACGATCATTACTGCCTCGATTACTGGGGCCAAG
GTACCACCTTAACAGTCTCCTCC.

### Anti-CD28 Antigen-Binding Domains

In some embodiments, the first target-binding domain, the second target-binding domain, and/or one or more of the one or more additional antigen-binding domains can be an anti-CD28 scFv. In some embodiments, the anti-CD28 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to:

(SEQ ID NO: 47)
DIEMTQSPAIMSASLGERVTMTCTASSSVSSSYFHWYQQKPGSSPKLCIY
STSNLASGVPPRFSGSGSTSYSLTISSMEAEDAATYFCHQYHRSPTFGGG
TKLETKR

and/or a light chain variable domain including a sequence that is at least 70% identical (e.g., at least 75% identical, at least 80% identical, at least 85% identical, at least 90% identical, at least 95% identical, at least 99% identical, or 100% identical) to:

(SEQ ID NO: 48)
VQLQQSGPELVKPGASVKMSCKASGYTFTSYVIQWVKQKPGQGLEWIGSI
NPYNDYTKYNEKFKGKATLTSDKSSITAYMEFSSLTSEDSALYYCARWGD
GNYWGRGTTLTVSS.

In some embodiments, the anti-CD28 scFv can include a heavy chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to:

(SEQ ID NO: 49)
GACATCGAGATGACACAGTCCCCCGCTATCATGAGCGCCTCTTTAGGAGA
ACGTGTGACCATGACTTGTACAGCTTCCTCCAGCGTGAGCAGCTCCTATT
TCCACTGGTACCAGCAGAAACCCGGCTCCTCCCCTAAACTGTGTATCTAC
TCCACAAGCAATTTAGCTAGCGGCGTGCCTCCTCGTTTTAGCGGCTCCGG
CAGCACCTCTTACTCTTTAACCATTAGCTCTATGGAGGCCGAAGATGCCG
CCACATACTTTTGCCATCAGTACCACCGGTCCCCTACCTTTGGCGGAGGC
ACAAAGCTGGAGACCAAGCGG,

and/or a light chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to:

(SEQ ID NO: 50)
GTGCAGCTGCAGCAGTCCGGACCCGAACTGGTCAAGCCCGGTGCCTCCGT
GAAAATGTCTTGTAAGGCTTCTGGCTACACCTTTACCTCCTACGTCATCC
AATGGGTGAAGCAGAAGCCCGGTCAAGGTCTCGAGTGGATCGGCAGCATC
AATCCCTACAACGATTACACCAAGTATAACGAAAAGTTTAAGGGCAAGGC
CACTCTGACAAGCGACAAGAGCTCCATTACCGCCTACATGGAGTTTTCCT
CTTTAACTTCTGAGGACTCCGCTTTATACTATTGCGCTCGTTGGGGCGAT
GGCAATTATTGGGGCCGGGGAACTACTTTAACAGTGAGCTCC.

In some embodiments, an anti-CD28 scFv can include a sequence that is at least 70% identical (e.g., at least 75% identical, at least 80% identical, at least 85% identical, at least 90% identical, at least 95% identical, at least 99% identical, or 100% identical) to:

(SEQ ID NO: 51)
VQLQQSGPELVKPGASVKMSCKASGYTFTSYVIQWVKQKPGQGLEWIGSI
NPYNDYTKYNEKFKGKATLTSDKSSITAYMEFSSLTSEDSALYYCARWGD
GNYWGRGTTLTVSSGGGGSGGGGSGGGGSDIEMTQSPAIMSASLGERVTM
TCTASSSVSSSYFHWYQQKPGSSPKLCIYSTSNLASGVPPRESGSGSTSY
SLTISSMEAEDAATYFCHQYHRSPTFGGGTKLETKR.

In some embodiments, an anti-CD28 scFv can include the six CDRs present in SEQ ID NO: 51.

In some embodiments, an anti-CD28 scFv can include a sequence encoded by a nucleic acid sequence that is at least 70% identical (e.g., at least 75% identical, at least 80% identical, at least 85% identical, at least 90% identical, at least 95% identical, at least 99% identical, or 100% identical) to:

(SEQ ID NO: 52)
GTGCAGCTGCAGCAGTCCGGACCCGAACTGGTCAAGCCCGGTGCCTCCGT
GAAAATGTCTTGTAAGGCTTCTGGCTACACCTTTACCTCCTACGTCATCC
AATGGGTGAAGCAGAAGCCCGGTCAAGGTCTCGAGTGGATCGGCAGCATC
AATCCCTACAACGATTACACCAAGTATAACGAAAAGTTTAAGGGCAAGGC
CACTCTGACAAGCGACAAGAGCTCCATTACCGCCTACATGGAGTTTTCCT
CTTTAACTTCTGAGGACTCCGCTTTATACTATTGCGCTCGTTGGGGCGAT
GGCAATTATTGGGGCCGGGGAACTACTTTAACAGTGAGCTCCGGCGGCGG
CGGAAGCGGAGGTGGAGGATCTGGCGGTGGAGGCAGCGACATCGAGATGA
CACAGTCCCCCGCTATCATGAGCGCCTCTTTAGGAGAACGTGTGACCATG
ACTTGTACAGCTTCCTCCAGCGTGAGCAGCTCCTATTTCCACTGGTACCA
GCAGAAACCCGGCTCCTCCCCTAAACTGTGTATCTACTCCACAAGCAATT
TAGCTAGCGGCGTGCCTCCTCGTTTTAGCGGCTCCGGCAGCACCTCTTAC
TCTTTAACCATTAGCTCTATGGAGGCCGAAGATGCCGCCACATACTTTTG
CCATCAGTACCACCGGTCCCCTACCTTTGGCGGAGGCACAAAGCTGGAGA
CCAAGCGG.

In some embodiments, the first target-binding domain, the second target-binding domain, and/or one or more of the one or more additional target-binding domains can be an anti-CD3 antibody. In some embodiments of any of the single-chain chimeric polypeptides described herein, the additional target-binding domain includes or is an anti-CD3 antibody.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 134)
QVQLVQSGGGVVQPGRSLRLSCKASGYTFTRYTMHWVRQAPGKGLEWIGY
INPSRGYTNYNQKVKDRFTISRDNSKNTAFLQMDSLRPEDIGVYFCARYY
DDHYCLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 135)
DIQMTQSPSSLSASVGDRVTITCSASSSVSYMNWYQQTPGKAPKRWIYDT
SKLASGVPSRFSGSGSGTDYTFTISSLOPEDIATYYCQQWSSNPFTFGQG
TKLQITRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSENRGEC.

In some embodiments of any of the single-chain chimeric polypeptides described herein, the additional target-binding domain includes or is an anti-CD3 antibody.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 136)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSAKTTAPSVYPLAPVCGGTTGSSVTLGCLVKG
YFPEPVTLTWNSGSLSSGVHTFPAVLQSDLYTLSSSVTVISSTWPSQSIT
CNVAHPASSTKVDKKIEPRPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 137)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRADTAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSENRNEC.

Additional examples and aspects of antigen-binding domains are known in the art.

### Anti-Tissue Factor Antigen-Binding Domain

In some embodiments, the first target-binding domain, the second target-binding domain, and/or one or more of the one or more additional target-binding domains can be an anti-tissue factor antigen-binding domain. In some embodiments of any of the single-chain chimeric polypeptides described herein, the additional target-binding domain includes or is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 98)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSS.

In some embodiments, an anti-tissue factor antibody can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 98)
DIQMTQSPASLSASVGDRVTITCLASQTIDTWLAWYLQKPGKSPQLLIY
AATNLADGVPSRFSGSGSGTDESFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSS,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 98)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSS.

In some embodiments, the anti-tissue factor antigen-binding domain can further include a linker sequence (e.g., any of the exemplary linker sequences described herein or known in the art) between the first sequence and the second sequence.

In some embodiments, the first target-binding domain, the second target-binding domain, and/or one or more of the one or more additional target-binding domains can be an anti-tissue factor diabody. In some embodiments of any of the single-chain chimeric polypeptides described herein, the additional target-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 99)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSSGGGGSGGGGSGGGGSD
IQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFG
QGTKLEIKGGGGSQIQLVQSGGEVKKPGASVRVSCKASGYSFTDYNVYW
VRQSPGKGLEWIGYIDPYNGITIYDQNFKGKATLTVDKSTSTAYMELSS
LRSEDTAVYFCARDVITALDFWGQGTTVTVSS.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 100)
GATATTCAGATGACACAGTCACCTGCCTCACTGTCAGCGTCTGTGGGGG
ACCGCGTGACAATTACGTGCCTGGCTAGCCAAACCATAGATACCTGGCT
GGCGTGGTACTTGCAGAAACCTGGAAAGTCGCCACAGCTGCTGATCTAT
GCGGCAACTAACCTGGCGGACGGCGTACCCTCGCGTTTTTCCGGGTCTG
GCAGCGGTACCGATTTCTCCTTCACTATTAGCAGCCTTCAGCCGGAGGA
CTTTGCTACATATTATTGTCAGCAAGTGTACTCATCCCCTTTCACCTTC
GGCCAAGGCACCAAACTAGAAATAAAAGGCGGGGGCGGTTCACAAATTC
AGCTGGTGCAAAGCGGCGGCGAAGTGAAGAAGCCTGGAGCTAGCGTGCG
TGTGAGCTGTAAGGCATCTGGCTACTCCTTCACTGATTACAACGTGTAT
TGGGTTAGACAGTCCCCTGGTAAAGGGCTTGAGTGGATTGGCTATATAG
ACCCTTATAACGGCATTACCATATACGATCAAAACTTCAAAGGTAAGGC
CACCCTGACCGTTGACAAGTCAACAAGCACCGCCTACATGGAGCTGAGC
AGCCTAAGAAGCGAGGACACCGCTGTCTATTTCTGTGCTAGAGACGTAA
CCACAGCCCTGGATTTCTGGGGCCAAGGCACAACGGTGACAGTCAGCTC
GGGTGGCGGCGGTTCGGGTGGGGGAGGAAGCGGGGGGGGGGGATCCGAC
ATTCAAATGACGCAAAGCCCTGCTAGCCTTTCCGCTAGCGTGGGAGACC
GCGTTACGATCACATGTCTGGCCTCTCAGACCATCGACACTTGGCTGGC
CTGGTACCTACAAAAACCTGGCAAAAGCCCACAACTACTGATCTATGCT
GCTACGAATCTGGCCGACGGCGTACCGTCCCGCTTCAGCGGCTCCGGTT
CTGGGACAGACTTCTCTTTCACCATTTCATCTCTGCAGCCTGAAGATTT
CGCCACATATTATTGTCAACAAGTGTATAGCAGCCCGTTTACCTTTGGG
CAAGGCACGAAGTTAGAAATCAAGGGCGGAGGCGGATCCCAAATTCAGC
TCGTGCAGAGTGGCGGGGAAGTCAAGAAACCTGGCGCTAGCGTGAGAGT
GTCTTGTAAGGCAAGTGGCTACAGCTTTACCGACTATAATGTGTATTGG
GTGAGACAATCACCCGGCAAGGGGCTGGAGTGGATCGGCTACATCGACC
CGTATAACGGCATTACGATCTACGACCAAAACTTTAAAGGCAAGGCCAC
CCTAACGGTCGACAAGTCGACCTCCACCGCTTATATGGAGCTTAGCTCA
TTGAGAAGCGAGGATACCGCGGTGTACTTCTGCGCTAGAGACGTGACAA
CCGCCCTGGACTTCTGGGGGCAAGGCACAACCGTCACCGTGTCGTCC.

In some embodiments, an anti-tissue factor antigen-binding domain can include a heavy chain variable sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 101)
QIQLVQSGGEVKKPGASVRVSCKASGYSFTDYNVYWVRQSPGKGLEWIG
YIDPYNGITIYDQNFKGKATLTVDKSTSTAYMELSSLRSEDTAVYFCAR
DVTTALDEWGQGTTVTVSS,

and a light chain variable sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 102)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIK.

In some embodiments, the first target-binding domain, the second target-binding domain, and/or one or more of the one or more additional target-binding domains can be an anti-tissue factor scFv. In some embodiments, the anti-tissue factor scFv is a humanized anti-human tissue factor scFv. In some embodiments, a humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 103)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSGGGGSGGGGSQIQLVQSGGEVKKPGASVRVSCKAS
GYSFTDYNVYWVRQSPGKGLEWIGYIDPYNGITIYDQNFKGKATLTVDK
STSTAYMELSSLRSEDTAVYFCARDVTTALDFWGQGTTVTVSS.

In some embodiments, an anti-tissue factor single chain antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 104)
GACATACAGATGACCCAAAGCCCTGCTAGCCTGAGCGCGAGCGTGGGCG
ACAGAGTGACCATCACCTGCCTCGCCTCTCAGACGATTGACACCTGGCT
GGCTTGGTATTTGCAGAAACCTGGGAAAAGCCCTCAGCTGCTGATCTAC
GCAGCCACCAATTTAGCCGATGGCGTCCCTTCTAGATTTAGCGGCTCGG
GATCCGGCACCGATTTTAGCTTTACAATTTCATCTCTACAGCCTGAGGA
CTTCGCCACCTATTATTGTCAACAAGTATATAGCAGCCCTTTCACCTTC
GGCCAAGGCACCAAGTTAGAGATCAAGGGCGGTGGCGGCTCCGGGGGCG
GGGGAAGTGGAGGTGGGGGGTCACAGATTCAGCTGGTTCAAAGCGGCGG
CGAGGTGAAGAAACCTGGCGCCTCGGTGAGAGTTAGCTGCAAAGCTAGC
GGATATTCGTTTACTGACTACAACGTGTATTGGGTGAGGCAGAGCCCGG
GCAAGGGACTGGAGTGGATTGGGTACATTGACCCCTACAACGGCATAAC
CATATATGATCAGAACTTCAAGGGCAAAGCCACGCTGACCGTGGATAAA
AGCACAAGCACCGCTTATATGGAGTTGAGCTCCCTCCGTAGCGAAGACA
CGGCAGTATACTTTTGCGCACGGGATGTGACCACGGCCCTAGATTTCTG
GGGACAAGGGACAACCGTTACTGTTAGTTCC.

### Anti-Tissue Factor and Anti-CD3 Binding Domains

In some embodiments, the first target-binding domain, the second target-binding domain, and/or one or more of the one or more additional target-binding domains includes both an anti-tissue factor domain and an anti-CD3 binding domain.

In some embodiments, a target-binding domain that includes both an anti-tissue factor domain and an anti-CD3 binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 105)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSGGGGSGGGGSQIQLVQSGGEVKKPGASVRVSCKAS
GYSFTDYNVYWVRQSPGKGLEWIGYIDPYNGITIYDQNFKGKATLTVDK
STSTAYMELSSLRSEDTAVYFCARDVTTALDFWGQGTTVTVSSGGGGSD
IQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFG
QGTKVEIKGGGGSGGGGSGGGGSEVQLVESGGGLVQPGGSLRLSCAASG
YSFTGYTMNWVRQAPGKGLEWVALINPYKGVSTYNQKFKDRFTISVDKS
KNTAYLQMNSLRAEDTAVYYCARSGYYGDSDWYFDVWGQGTLVTVSSGG
GGS.

In some embodiments, a target-binding domain that includes both an anti-tissue factor domain and an anti-CD3 binding domain is encoded by a nucleic acid including a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 106)
GACATACAGATGACCCAAAGCCCTGCTAGCCTGAGCGCGAGCGTGGGCG
ACAGAGTGACCATCACCTGCCTCGCCTCTCAGACGATTGACACCTGGCT
GGCTTGGTATTTGCAGAAACCTGGGAAAAGCCCTCAGCTGCTGATCTAC
GCAGCCACCAATTTAGCCGATGGCGTCCCTTCTAGATTTAGCGGCTCGG
GATCCGGCACCGATTTTAGCTTTACAATTTCATCTCTACAGCCTGAGGA
CTTCGCCACCTATTATTGTCAACAAGTATATAGCAGCCCTTTCACCTTC
GGCCAAGGCACCAAGTTAGAGATCAAGGGCGGTGGCGGCTCCGGGGGCG
GGGGAAGTGGAGGTGGGGGGTCACAGATTCAGCTGGTTCAAAGCGGCGG
CGAGGTGAAGAAACCTGGCGCCTCGGTGAGAGTTAGCTGCAAAGCTAGC
GGATATTCGTTTACTGACTACAACGTGTATTGGGTGAGGCAGAGCCCGG
GCAAGGGACTGGAGTGGATTGGGTACATTGACCCCTACAACGGCATAAC
CATATATGATCAGAACTTCAAGGGCAAAGCCACGCTGACCGTGGATAAA
AGCACAAGCACCGCTTATATGGAGTTGAGCTCCCTCCGTAGCGAAGACA
CGGCAGTATACTTTTGCGCACGGGATGTGACCACGGCCCTAGATTTCTG
GGGACAAGGGACAACCGTTACTGTTAGTTCCGGCGGTGGGGGCTCGGAT
ATTCAGATGACGCAGTCGCCAAGCTCACTCTCGGCCTCCGTAGGAGACA
GAGTTACCATCACCTGCAGAGCTAGCCAAGACATCAGAAACTACCTGAA
CTGGTATCAGCAGAAGCCTGGCAAGGCCCCTAAGCTCCTGATCTACTAC
ACGAGCAGACTTGAGAGCGGGGTACCATCCCGCTTCAGCGGATCTGGAA
GCGGCACAGACTACACCCTGACCATCTCTAGTCTGCAGCCTGAGGACTT
CGCCACTTACTATTGTCAGCAAGGCAACACCCTGCCTTGGACCTTTGGC
CAAGGCACCAAAGTTGAGATAAAGGGAGGGGGAGGGAGCGGTGGCGGAG
GCTCGGGTGGGGGTGGCTCAGAAGTACAGCTGGTTGAGTCCGGCGGTGG
TCTGGTGCAGCCTGGCGGAAGCCTGAGACTGAGCTGCGCCGCAAGCGGC
TATAGCTTCACTGGCTATACCATGAACTGGGTGCGCCAAGCCCCTGGCA
AGGGCCTGGAATGGGTGGCCCTGATCAACCCTTACAAGGGCGTGAGCAC
CTACAATCAGAAGTTCAAGGACAGATTCACCATCAGCGTGGACAAGAGC
AAGAACACCGCCTACCTGCAGATGAACAGCCTGAGAGCCGAGGACACTG
CGGTCTATTACTGTGCTAGAAGCGGCTACTACGGCGACAGCGACTGGTA
CTTCGACGTGTGGGGGCAAGGCACCTTGGTAACGGTAAGCTCCGGCGGT
GGGGGCTCG.

### Anti-PD-1 Antigen-Binding Domains

In some embodiments, the first target-binding domain, the second target-binding domain, and/or one or more of the one or more additional target-binding domains can be an anti-PD-1 antigen-binding domain. In some embodiments of any of the single-chain chimeric polypeptides described herein, the additional target-binding domain includes or is an anti-PD-1 antigen-binding domain.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 107)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 107)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 107)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, the anti-PD-1 antigen-binding domain is a humanized anti-human PD-1 diabody. In some embodiments, a humanized anti-human PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 108)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIKGGGGSGGGG
SGGGGSQVQLVQSGVEVKKPGASVKVSCKASGYTFTNYYMYWVRQAPGQ
GLEWMGGINPSNGGTNFNEKFKNRVTLTTDSSTTTAYMELKSLQFDDTA
VYYCARRDYRFDMGFDYWGQGTTVTVSSGGGGSEIVLTQSPATLSLSPG
ERATLSCRASKGVSTSGYSYLHWYQQKPGQAPRLLIYLASYLESGVPAR
FSGSGSGTDFTLTISSLEPEDFAVYYCQHSRDLPLTFGGGTKVEIK.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 109)
CAAGTGCAGCTGGTGCAGAGCGGCGTGGAGGTGAAGAAGCCGGGCGCTAGC
GTGAAGGTGAGCTGCAAGGCCTCCGGATATACCTTCACAAACTACTACATG
TATTGGGTTAGACAAGCCCCTGGTCAAGGCCTGGAGTGGATGGGCGGTATA
AACCCTTCCAATGGAGGTACTAATTTCAACGAAAAATTCAAGAACCGTGTG
ACCCTGACCACCGACAGTAGCACCACCACTGCATACATGGAGCTGAAGAGT
CTGCAGTTCGACGATACCGCGGTCTACTACTGTGCCCGACGAGACTATAGA
TTTGACATGGGCTTCGACTACTGGGGACAAGGCACTACGGTGACCGTGAGC
AGCGGTGGCGGCGGCTCCGAAATTGTGCTGACACAGTCGCCTGCAACCCTG
TCCTTGAGCCCTGGCGAGCGGGCAACTCTGAGCTGCCGAGCTAGCAAAGGC
GTGAGTACAAGCGGCTATAGCTACCTACATTGGTATCAGCAAAAGCCTGGC
CAAGCCCCGCGACTGCTGATCTACCTGGCTAGCTACCTGGAGAGCGGCGTT
CCTGCTAGATTCAGCGGATCTGGCTCTGGTACTGACTTCACCTTAACTATC
AGTAGCCTTGAACCCGAGGACTTCGCTGTGTACTATTGTCAGCACAGCAGA
GACCTCCCTCTGACCTTTGGAGGTGGTACCAAGGTCGAAATCAAAGGAGGC
GGAGGGTCGGGCGGTGGTGGGTCTGGAGGTGGAGGAAGCCAAGTTCAACTC
GTGCAGAGCGGTGTGGAAGTTAAAAAGCCCGGCGCTAGCGTAAAGGTGAGC
TGTAAGGCTAGCGGCTACACCTTCACCAATTACTACATGTACTGGGTTAGG
CAAGCCCCTGGACAAGGCTTAGAATGGATGGGTGGGATAAACCCTTCGAAT
GGTGGAACCAACTTTAACGAGAAGTTCAAAAACAGAGTGACGTTGACCACC
GACTCTAGCACCACTACCGCGTACATGGAACTGAAGAGCCTGCAGTTCGAT
GATACAGCGGTGTATTATTGCGCCCGAAGAGACTACCGATTCGACATGGGG
TTTGACTACTGGGGCCAAGGCACCACCGTCACCGTCAGCAGCGGCGGAGGC
GGTAGTGAGATTGTTCTGACCCAAAGCCCTGCCACCCTCTCGTTAAGCCCG
GGGGAGAGAGCAACCCTGAGCTGTAGAGCTAGCAAGGGCGTGAGCACCTCG
GGCTACAGTTACCTGCACTGGTACCAACAGAAGCCTGGCCAAGCCCCTCGA
TTATTGATCTACTTAGCTAGCTACTTAGAAAGCGGCGTGCCCGCTAGATTC
AGCGGCAGCGGGAGCGGCACCGACTTTACACTGACGATAAGTAGCTTAGAG
CCTGAGGACTTTGCAGTGTATTACTGCCAACATTCCCGGGACCTGCCGCTT
ACCTTCGGGGGCGGCACCAAAGTGGAGATTAAA.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 110)
QVQLQQSGAELVKPGSSVKISCKASGYTFTSHFIHWIKQQPGNGLEWIGGI
YPGDGDTEYNQQFNGKATLTADKSSSTAYMRLSSLTSEDSAVYFCATRVPS
YWFFDFWGPGTMVTVSSGGGGSRTDVALTQTPVAQPVTLGDQASISCRSSQ
SLVHSNGRTYLEWYLQKPGQSPOLLIYKVSNRFSGVPDRFIGSGSGSDFTL
TISRVEPEDLGVYYCFQATHDPNTFGAGTKLELKGGGGSGGGGSGGGGSQV
QLQQSGAELVKPGSSVKISCKASGYTFTSHFIHWIKQQPGNGLEWIGGIYP
GDGDTEYNQQENGKATLTADKSSSTAYMRLSSLTSEDSAVYFCATRVPSYW
FFDFWGPGTMVTVSSGGGGSRTDVALTQTPVAQPVTLGDQASISCRSSQSL
VHSNGRTYLEWYLQKPGQSPQLLIYKVSNRFSGVPDRFIGSGSGSDFTLTI
SRVEPEDLGVYYCFQATHDPNTFGAGTKLELK.

In some embodiments, the anti-PD-1 antigen-binding domain is an anti-PD-1 scFv. In some embodiments, the anti-PD-1 antigen-binding domain is an anti-human PD-1 scFv. In some embodiments, an anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 111)
EIVLTQSPATLSLSPGERATLSCRASKGVSTSGYSYLHWYQQKPGQAPRLL
IYLASYLESGVPARFSGSGSGTDFTLTISSLEPEDFAVYYCQHSRDLPLTF
GGGTKVEIKGGGGSGGGGSGGGGSQVQLVQSGVEVKKPGASVKVSCKASGY
TFTNYYMYWVRQAPGQGLEWMGGINPSNGGTNENEKFKNRVTLTTDSSTTT
AYMELKSLQFDDTAVYYCARRDYRFDMGFDYWGQGTTVTVSS.

In some embodiments, the anti-PD-1 antigen-binding domain is an anti-mouse PD-1 scFv. In some embodiments, an anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 112)
RTDVALTQTPVAQPVTLGDQASISCRSSQSLVHSNGRTYLEWYLQKPGQSP
QLLIYKVSNRFSGVPDRFIGSGSGSDFTLTISRVEPEDLGVYYCFQATHDP
NTFGAGTKLELKGGGGSGGGGSGGGGSQVQLQQSGAELVKPGSSVKISCKA
SGYTFTSHEIHWIKQQPGNGLEWIGGIYPGDGDTEYNQQENGKATLTADKS
SSTAYMRLSSLTSEDSAVYFCATRVPSYWFFDFWGPGTMVTVSS.

In some embodiments, the first target-binding domain, the second target-binding domain, and/or one or more of the one or more additional target-binding domains can be an anti-PD-1 antibody. In some embodiments of any of the single-chain chimeric polypeptides described herein, the additional target-binding domain includes or is an anti-PD-1 antibody.

In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 113)
QVQLVQSGVEVKKPGASVKVSCKASGYTFTNYYMYWVRQAPGQGLEWMGGI
NPSNGGTNFNEKFKNRVTLTTDSSTTTAYMELKSLQFDDTAVYYCARRDYR
FDMGEDYWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYS
RLTVDKSRWQEGNVESCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 114)
EIVLTQSPATLSLSPGERATLSCRASKGVSTSGYSYLHWYQQKPGQAPRLL
IYLASYLESGVPARFSGSGSGTDFTLTISSLEPEDFAVYYCQHSRDLPLTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSENRGEC.

In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 1175
QVQLVQSGAEVKKPGASVKVSCKASGYSFTSYWMNWVRQAPGQGLEWIGVI
HPSDSETWLDQKFKDRVTITVDKSTSTAYMELSSLRSEDTAVYYCAREHYG
TSPFAYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSR
LTVDKSRWQEGNVFSCSVMHEALHNHYTQKSLSLSLG,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 116)
EIVLTQSPATLSLSPGERATLSCRASESVDNYGMSFMNWFQQKPGQPPKLL
IHAASNQGSGVPSRFSGSGSGTDFTLTISSLEPEDFAVYFCQQSKEVPYTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSENRGEC.

In some embodiments, the first target-binding domain, the second target-binding domain, and/or one or more of the one or more additional target-binding domains can be an anti-PD-1 antigen-binding domain. In some embodiments of any of the single-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 117)
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPCSRSTSESTAALGCLVKDYFPEPVTVS
WNSGALTSGVHTFPAVLQSSGLYSLSSVVTVPSSSLGTKTYTCNVDHKPSN
TKVDKRVESKYGPPCPPCPAPEFLGGPSVFLFPPKPKDTLMISRTPEVTCV
VVDVSQEDPEVQENWYVDGVEVHNAKTKPREEQFNSTYRVVSVLTVLHQDW
LNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKS
RWQEGNVFSCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 118)
EIVLTQSPATLSLSPGERATLSCRASQSVSSYLAWYQQKPGQAPRLLIYDA
SNRATGIPARFSGSGSGTDFTLTISSLEPEDFAVYYCQQSSNWPRTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSENRGEC.

In some embodiments, the first target-binding domain, the second target-binding domain, and/or one or more of the one or more additional target-binding domains can be an anti-PD-1 antigen-binding domain. In some embodiments of any of the single-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 119)
EVQLLESGGGLVQPGGSLRLSCAASGFTFSSYDMSWVRQAPGKGLEWVSTI
SGGGSYTYYQDSVKGRFTISRDNSKNTLYLQMNSLRAEDTAVYYCASPYYA
MDYWGQGTTVTVSSASTKGPSVFPLAPCSRSTSESTAALGCLVKDYFPEPV
TVSWNSGALTSGVHTFPAVLQSSGLYSLSSVVTVPSSSLGTKTYTCNVDHK
PSNTKVDKRVESKYGPPCPPCPAPEFLGGPSVFLFPPKPKDTLMISRTPEV
TCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTV
DKSRWQEGNVFSCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 120)
DIQLTQSPSFLSAYVGDRVTITCKASQDVGTAVAWYQQKPGKAPKLLIYWA
STLHTGVPSRFSGSGSGTEFTLTISSLQPEDFATYYCQHYSSYPWTFGQGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSENRGEC.

In some embodiments, the first target-binding domain, the second target-binding domain, and/or one or more of the one or more additional target-binding domains can be an anti-PD-1 antigen-binding domain. In some embodiments of any of the single-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 121)
EVQLLESGGVLVQPGGSLRLSCAASGFTFSNFGMTWVRQAPGKGLEWVSGI
SGGGRDTYFADSVKGRFTISRDNSKNTLYLQMNSLKGEDTAVYYCVKWGNI
YFDYWGQGTLVTVSSASTKGPSVFPLAPCSRSTSESTAALGCLVKDYFPEP
VTVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLT
VDKSRWQEGNVFSCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 122)
DIQMTQSPSSLSASVGDSITITCRASLSINTELNWYQQKPGKAPNLLIYAA
SSLHGGVPSRFSGSGSGTDFTLTIRTLQPEDFATYYCQQSSNTPFTFGPGT
VVDFRRTVAAPSVFIFPPSDEQLKSGTASVVCLLNNFYPREAKVQWKVDNA
LQSGNSQESV.

In some embodiments, the first target-binding domain, the second target-binding domain, and/or one or more of the one or more additional target-binding domains can be an anti-PD-1 antigen-binding domain. In some embodiments of any of the single-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 123)
QGQLVQSGAEVKKPGASVKVSCKASGYTFTDYEMHWVRQAPIHGLEWIGVI
ESETGGTAYNQKFKGRVTITADKSTSTAYMELSSLRSEDTAVYYCAREGIT
TVATTYYWYFDVWGQGTTVTVSSASTKGPSVFPLAPCSRSTSESTAALGCL
VKDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 124)
DVVMTQSPLSLPVTLGQPASISCRSSQSIVHSNGNTYLEWYLQKPGQSP
QLLIYKVSNRFSGVPDRFSGSGSGTDFTLKISRVEAEDVGVYYCFQGSH
VPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

### Anti-CD16a Antigen-Binding Domains

In some embodiments, the first target-binding domain, the second target-binding domain, and/or one or more of the one or more additional target-binding domains can be an anti-CD16a antigen-binding domain. In some embodiments of any of the single-chain chimeric polypeptides described herein, the additional target-binding domain includes or is an anti-CD16a antigen-binding domain.

In some embodiments, the anti-CD16a antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 125)
SELTQDPAVSVALGQTVRITCQGDSLRSYYASWYQQKPGQAPVLVIYGK
NNRPSGIPDRFSGSSSGNTASLTITGAQAEDEADYYCNSRDSSGNHVVF
GGGTKLTVGHGGGGSGGGGSGGGGSEVQLVESGGGVVRPGGSLRLSCAA
SGFTFDDYGMSWVRQAPGKGLEWVSGINWNGGSTGYADSVKGRFTISRD
NAKNSLYLQMNSLRAEDTAVYYCARGRSLLFDYWGQGTLVTVSR.

In some embodiments, the anti-CD16a antigen-binding domain is encoded by a nucleic acid that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 126)
AGCGAGCTGACCCAAGACCCTGCCGTGAGCGTGGCCCTGGGACAGACCG
TGAGAATCACCTGCCAAGGCGACAGCCTGAGAAGCTACTACGCTAGCTG
GTATCAGCAGAAGCCTGGCCAAGCCCCTGTGCTGGTGATCTACGGCAAG
AACAACAGACCTAGCGGCATCCCTGACAGATTCAGCGGCAGCAGCAGCG
GCAACACCGCTAGCCTGACCATCACCGGCGCCCAAGCCGAGGACGAGGC
CGACTACTACTGCAACAGCAGAGACAGCAGCGGCAACCACGTGGTCTTC
GGCGGAGGAACCAAACTCACCGTCGGCCACGGCGGAGGCGGAAGCGGCG
GCGGAGGCTCCGGCGGAGGCGGCAGCGAAGTGCAGCTGGTGGAGAGCGG
AGGAGGCGTCGTCAGACCTGGCGGCAGCCTGAGACTGAGCTGCGCCGCT
AGCGGCTTCACCTTCGACGACTACGGCATGAGCTGGGTGAGACAAGCCC
CTGGCAAGGGCCTGGAGTGGGTGAGCGGCATCAACTGGAACGGCGGCAG
CACCGGCTACGCCGACAGCGTGAAGGGCAGATTCACCATCAGCAGAGAC
AACGCCAAGAACAGCCTGTACCTGCAGATGAACAGCCTGAGAGCCGAGG
ACACCGCCGTGTACTACTGCGCTAGAGGCAGAAGCCTGCTGTTCGACTA
CTGGGGCCAAGGCACCCTGGTGACCGTGAGCAGA.

### Anti-HIV Antigen-Binding Domains

In some embodiments, the first target-binding domain, the second target-binding domain, and/or one or more of the one or more additional target-binding domains can be an anti-HIV antigen-binding domain. In some embodiments of any of the single-chain chimeric polypeptides described herein, the additional target-binding domain includes or is an anti-HIV antigen-binding domain.

In some embodiments, the anti-HIV antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 127)
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGGGGSGGGGSGGGGSQVQLQESGPGLVKPSETLSVTCSV
SGDSMNNYYWTWIRQSPGKGLEWIGYISDRESATYNPSLNSRVVISRDT
SKNQLSLKLNSVTPADTAVYYCATARRGQRIYGVVSFGEFFYYYSMDVW
GKGTTVTVSS.

In some embodiments, the anti-HIV antigen-binding domain is encoded by a nucleic acid that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 128)
TCTTATGTAAGACCGCTCTCCGTCGCATTGGGGGAGACCGCGCGCATTA
GTTGTGGACGACAAGCACTTGGATCTCGAGCAGTTCAGTGGTACCAGCA
CCGACCCGGACAAGCTCCTATTCTGCTCATTTATAATAACCAAGACAGA
CCATCAGGCATCCCCGAGCGATTCTCTGGGACCCCGGATATCAATTTCG
GAACCAGGGCGACACTTACTATTTCAGGTGTTGAGGCCGGGGACGAGGC
AGACTATTACTGCCACATGTGGGACAGCCGATCTGGATTTTCTTGGTCA
TTTGGAGGCGCAACACGCTTGACCGTTCTCGGAGGGGGTGGCTCAGGAG
GCGGCGGATCAGGAGGGGGAGGTTCACAAGTGCAACTTCAGGAATCCGG
ACCTGGACTCGTGAAGCCTTCAGAAACCCTTTCAGTAACGTGCAGCGTG
AGCGGAGATAGCATGAACAACTATTACTGGACTTGGATAAGACAATCTC
CGGGCAAGGGGCTCGAGTGGATAGGGTATATCTCAGATAGAGAATCCGC
CACTTATAATCCTAGCCTTAACAGCCGGGTAGTCATAAGCAGAGACACA
AGCAAAAATCAACTCAGTTTGAAATTGAATTCAGTAACACCTGCCGACA
CCGCAGTGTACTATTGCGCGACAGCCCGGCGAGGGCAGAGGATTTATGG
CGTCGTTAGCTTTGGAGAATTTTTCTACTATTACTCCATGGATGTGTGG
GGCAAGGGGACCACGGTTACTGTGTCTTCA.

Additional examples and aspects of antigen-binding domains are known in the art.

### Additional Target-Binding Domains

Some embodiments of any of the single-chain chimeric polypeptides described herein can further include one or more (e.g., two, three, four, five, six, seven, eight, nine, or ten) additional target-binding domains (e.g., any of the exemplary target-binding domains described herein or known in the art) at its N- and/or C-terminus.

In some embodiments, the single-chain chimeric polypeptides can include one or more (e.g., two, three, four, five, six, seven, eight, nine, or ten) additional target-binding domains (e.g., any of the exemplary target-binding domains described herein or known in the art) at its N-terminus. In some embodiments, one of the one or more additional target-binding domains (e.g., any of the exemplary target-binding domains described herein or known in the art) at the N-terminus of the single-chain chimeric polypeptide can directly abut the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein). In some embodiments, the single-chain chimeric polypeptide further includes a linker sequence (e.g., any of the exemplary linker sequences described herein or known in the art) between one of the at least one additional target-binding domains (e.g., any of the exemplary target-binding domains described herein or known in the art) at the N-terminus of the single-chain chimeric polypeptide and the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein).

In some embodiments of any of the single-chain chimeric polypeptides described herein, the single-chain chimeric polypeptide includes one or more (e.g., two, three, four, five, six, seven, eight, nine, or ten) additional target-binding domains (e.g., any of the exemplary target-binding domains described herein or known in the art) at its C-terminus. In some embodiments, one of the one or more additional target-binding domains (e.g., any of the exemplary target-binding domains described herein or known in the art) at the C-terminus of the single-chain chimeric polypeptide directly abuts the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein or known in the art). In some embodiments, the single-chain chimeric polypeptide further comprises a linker sequence (e.g., any of the exemplary linker sequences described herein or known in the art) between one of the at least one additional target-binding domains (e.g., any of the exemplary target-binding domains described herein or known in the art) at the C-terminus of the single-chain chimeric polypeptide and the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein).

In some embodiments of any of the single-chain chimeric polypeptides described herein, the single-chain chimeric polypeptide comprises one or more (e.g., two, three, four, five, six, seven, eight, nine, or ten) additional target-binding domains (e.g., any of the exemplary target-binding domains described herein or known in the art) at its N-terminus and its C-terminus. In some embodiments, one of the one or more additional target-binding domains (e.g., any of the exemplary target-binding domains described herein or known in the art) at the N-terminus of the single-chain chimeric polypeptide directly abuts the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein). In some embodiments, the single-chain chimeric polypeptide further includes a linker sequence (e.g., any of the exemplary linker sequences described herein or known in the art) between one of the one or more additional target-binding domains (e.g., any of the exemplary target-binding domains described herein or known in the art) at the N-terminus and the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains). In some embodiments, one of the one or more additional target-binding domains (e.g., any of the exemplary target-binding domains described herein or known in the art) at the C-terminus directly abuts the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains). In some embodiments, the single-chain chimeric polypeptide further includes a linker sequence (e.g., any of the exemplary linker sequences described herein or known in the art) between one of the one or more additional target-binding domains (e.g., any of the exemplary target-binding domains described herein or known in the art) at the C-terminus and the first target-binding domain (e.g., any of the exemplary target-binding domains described herein or known in the art), the second target-binding domain (e.g., any of the exemplary target-binding domains described herein or known in the art), or the soluble TRβC domain (e.g., any of the exemplary soluble TRβC domains described herein).

In some embodiments of any of the single-chain chimeric polypeptides described herein, two or more (e.g., three, four, five, six, seven, eight, nine, or ten) of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) bind specifically to the same antigen. In some embodiments, two or more (e.g., three, four, five, six, seven, eight, nine, or ten) of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) bind specifically to the same epitope. In some embodiments, two or more (e.g., three, four, five, six, seven, eight, nine, or ten) of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) include the same amino acid sequence.

In some embodiments of any of the single-chain chimeric polypeptides described herein, the first target-binding domain (e.g., any of the exemplary target-binding domains described herein or known in the art), the second target-binding domain (e.g., any of the exemplary target-binding domains described herein or known in the art), and the one or more (e.g., two, three, four, five, six, seven, eight, nine, or ten) additional target-binding domains (e.g., any of the exemplary target-binding domains described herein or known in the art) each bind specifically to the same antigen. In some embodiments, the first target-binding domain (e.g., any of the exemplary target-binding domains described herein or known in the art), the second target-binding domain (e.g., any of the exemplary target-binding domains described herein or known in the art), and the one or more (e.g., two, three, four, five, six, seven, eight, nine, or ten) additional target-binding domains (e.g., any of the exemplary target-binding domains described herein or known in the art) each bind specifically to the same epitope. In some embodiments, the first target-binding domain, the second target-binding domain, and the one or more (e.g., two, three, four, five, six, seven, eight, nine, or ten) additional target-binding domains each comprise the same amino acid sequence.

In some embodiments of any of the single-chain chimeric polypeptides described herein, the first target-binding domain (e.g., any of the exemplary target-binding domains described herein or known in the art), the second target-binding domain (e.g., any of the exemplary target-binding domains described herein or known in the art), and the one or more (e.g., two, three, four, five, six, seven, eight, nine, or ten) additional target-binding domains (e.g., any of the exemplary target-binding domains described herein or known in the art) bind specifically to different antigens.

In some embodiments of any of the single-chain chimeric polypeptides, one or more of the first target-binding domain, the second target-binding domain, and the one or more target-binding domains is an antigen-binding domain (e.g., any of the exemplary antigen-binding domains described herein or known in the art). In some embodiments of any of the single-chain chimeric polypeptides described herein, the first target-binding domain, the second target-binding domain, and the one or more additional target-binding domains are each an antigen-binding domain (e.g., any of the exemplary antigen-binding domains described herein or known in the art). In some embodiments, the antigen-binding domain can include a scFv or a single domain antibody.

In some embodiments of any of the single-chain chimeric polypeptides described herein, one or more (e.g., two, three, four, five, six, seven, eight, nine, or ten) of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) bind specifically to a target independently selected from the group consisting of: tissue factor (CD142), CD16a, CD28, CD3, CD33, CD20, CD19, CD22, CD123, IL-1R, IL-1, VEGF, IL-6R, IL-4, IL-10, PDL-1, TIGIT, PD-1, LAC-3, TIM3, CTLA4, MICA, MICB, IL-6, IL-8, TNFα, CD26a, CD36, ULBP2, CD30, CD40, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein, HLA-DR, DLL4, TYRO3, AXL, MER, CD122, CD155, PDGF-DD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand for ICOS, a receptor for IL-1, a receptor for IL-2, a receptor for IL-3, a receptor for IL-4, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of any of the single-chain chimeric polypeptides described herein, one or more of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) is a soluble interleukin protein, a soluble cytokine protein, or a soluble cell surface protein. Non-limiting examples of soluble interleukin proteins and soluble cytokine proteins include: IL-1, IL-2, IL-3, IL-4, IL-7, IL-8, IL-10, IL-12, IL-15, IL-17, IL-18, IL-21, PDGF-DD, SCF, and FLT3L. Non-limiting examples of soluble cell surface proteins include: MICA, MICB, and a ULP16-binding protein.

In some embodiments of any of the single-chain chimeric polypeptides described herein, one or more of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) is a soluble interleukin receptor, a soluble cytokine receptor, or a soluble cell surface receptor. Non-limiting examples of soluble interleukin receptors and soluble cytokine receptors include: a soluble TGF-β receptor II (TGF-βRII) and a soluble TGF-βRIII. Non-limiting examples of soluble cell surface receptors include: a soluble NKG2D, a soluble NK30, a soluble NKp44, a soluble NKp46, a soluble DNAM1, a scMHCI, a scMHCII, a scTCR, a soluble CD155, a soluble CD122, a soluble CD3, and a soluble CD28.

### Linker Sequences

In some embodiments of any of the single-chain chimeric polypeptides described herein, the single-chain chimeric polypeptide can include one, two, three, four, five, six, seven, eight, nine, or ten linker sequence(s) (e.g., the same or different linker sequences, e.g., any of the exemplary linker sequences described herein or known in the art). In some embodiments of any of the single-chain chimeric polypeptides described herein, the single-chain chimeric polypeptide can include one, two, three, four, five, six, seven, eight, nine, or ten linker sequence(s) (e.g., the same or different linker sequences, e.g., any of the exemplary linker sequences described herein or known in the art).

In some embodiments of any of the single-chain chimeric polypeptides described herein, the linker sequence can be a flexible linker sequence. Non-limiting examples of linker sequences that can be used are described in Klein et al., Protein Engineering, Design & Selection Vol. 27, No. 10, pp. 325-330, 2014; Priyanka et al., Protein Sci., 2013 February; 22(2): 153-167. In some examples, the linker sequence is a synthetic linker sequence.

In some embodiments, a linker sequence can have a total length of 1 amino acid to about 100 amino acids, 1 amino acid to about 90 amino acids, 1 amino acid to about 80 amino acids, 1 amino acid to about 70 amino acids, 1 amino acid to about 60 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 90 amino acids, about 2 amino acids to about 80 amino acids, about 2 amino acids to about 70 amino acids, about 2 amino acids to about 60 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 90 amino acids, about 4 amino acids to about 80 amino acids, about 4 amino acids to about 70 amino acids, about 4 amino acids to about 60 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 90 amino acids, about 6 amino acids to about 80 amino acids, about 6 amino acids to about 70 amino acids, about 6 amino acids to about 60 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 90 amino acids, about 8 amino acids to about 80 amino acids, about 8 amino acids to about 70 amino acids, about 8 amino acids to about 60 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 100 amino acids, about 10 amino acids to about 90 amino acids, about 10 amino acids to about 80 amino acids, about 10 amino acids to about 70 amino acids, about 10 amino acids to about 60 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 100 amino acids, about 12 amino acids to about 90 amino acids, about 12 amino acids to about 80 amino acids, about 12 amino acids to about 70 amino acids, about 12 amino acids to about 60 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 100 amino acids, about 14 amino acids to about 90 amino acids, about 14 amino acids to about 80 amino acids, about 14 amino acids to about 70 amino acids, about 14 amino acids to about 60 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 100 amino acids, about 16 amino acids to about 90 amino acids, about 16 amino acids to about 80 amino acids, about 16 amino acids to about 70 amino acids, about 16 amino acids to about 60 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 100 amino acids, about 18 amino acids to about 90 amino acids, about 18 amino acids to about 80 amino acids, about 18 amino acids to about 70 amino acids, about 18 amino acids to about 60 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 24 amino acids, about 18 amino acids to about 22 amino acids, about 18 amino acids to about 20 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 24 amino acids, about 20 amino acids to about 22 amino acids, about 22 amino acids to about 100 amino acids, about 22 amino acids to about 90 amino acids, about 22 amino acids to about 80 amino acids, about 22 amino acids to about 70 amino acids, about 22 amino acids to about 60 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 22 amino acids to about 24 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 100 amino acids, about 60 amino acids to about 90 amino acids, about 60 amino acids to about 80 amino acids, about 60 amino acids to about 70 amino acids, about 70 amino acids to about 100 amino acids, about 70 amino acids to about 90 amino acids, about 70 amino acids to about 80 amino acids, about 80 amino acids to about 100 amino acids, about 80 amino acids to about 90 amino acids, or about 90 amino acids to about 100 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences.

In some embodiments, the linker sequence comprises or consists of GGGGSGGGGSGGGGS (SEQ ID NO: 53). In some embodiments, the linker sequence is encoded by a nucleic acid comprising or consisting of:

(SEQ ID NO: 54)
GGCGGTGGAGGATCCGGAGGAGGTGGCTCCGGCGGCGGAGGATCT.

In some embodiments, the linker sequence comprises or consists of: GGGSGGGS (SEQ ID NO: 55). In some embodiments, the linker sequence comprises or consists of: GGGS (SEQ ID NO: 56).

### Signal Sequences

In some embodiments, a single-chain chimeric polypeptide includes a signal sequence at its N-terminal end. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a single-chain chimeric polypeptide includes a signal sequence having an amino acid sequence:

(SEQ ID NO: 57)
MKWVTFISLLELESSAYS.

In some embodiments, a single chain chimeric polypeptide includes a signal sequence encoded by the nucleic acid sequence:

(SEQ ID NO: 58)
ATGAAATGGGTGACCTTTATTTCTTTACTGTTCCTCTTTAGCAGCGC
CTACTCC,
or
(SEQ ID NO: 59)
ATGAAGTGGGTCACATTTATCTCTTTACTGTTCCTCTTCTCCAGCGC
CTACAGC.

In some embodiments, a single-chain chimeric polypeptide includes a signal sequence having an amino acid sequence:

(SEQ ID NO: 60)
MKCLLYLAFLFLGVNC.

In some embodiments, a single-chain chimeric polypeptide includes a signal sequence having an amino acid sequence:

(SEQ ID NO: 61)
MGQIVTMFEALPHIIDEVINIVIIVLIIITSIKAVYNFATCGILALV
SFLFLAGRSCG.

In some embodiments, a single-chain chimeric polypeptide includes a signal sequence having an amino acid sequence:

(SEQ ID NO: 62)
MPNHQSGSPTGSSDLLLSGKKQRPHLALRRKRRREMRKINRKVRRMN
LAPIKEKTAWQHLQALISEAEEVLKTSQTPQNSLTLFLALLSVLGPP
VTG.

In some embodiments, a single-chain chimeric polypeptide includes a signal sequence having an amino acid sequence:

(SEQ ID NO: 63)
MDSKGSSQKGSRLLLLLVVSNLLLCQGVVS.

In some embodiments, a first chimeric polypeptide of a single-chain chimeric polypeptide, a second chimeric polypeptide of the single-chain chimeric polypeptide, or both includes a signal sequence having an amino acid sequence:

(SEQ ID NO: 129)
MGVKVLFALICIAVAEA.

In some embodiments, a first chimeric polypeptide of a single-chain chimeric polypeptide, a second chimeric polypeptide of the single-chain chimeric polypeptide, or both includes a signal sequence having an amino acid sequence:

(SEQ ID NO: 130)
MERTLVCLVVIFLGTVA.

In some embodiments, a first chimeric polypeptide of a single-chain chimeric polypeptide, a second chimeric polypeptide of the single-chain chimeric polypeptide, or both includes a signal sequence having an amino acid sequence:

(SEQ ID NO: 131)
MGWSCIILFLVATATGVHS.

Those of ordinary skill in the art will be aware of other appropriate signal sequences for use in a single-chain chimeric polypeptide.

In some embodiments, a single-chain chimeric polypeptide includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKWVTFISLLFLFSSAYS (SEQ ID NO: 57) can include one or more additional amino acids at the N-terminus or C-terminus, while still retaining the ability to direct the single-chain chimeric polypeptide to the secretory pathway.

In some embodiments, a single-chain chimeric polypeptide includes a signal sequence that directs the single-chain chimeric polypeptide into the extracellular space. Such embodiments are useful in producing single-chain chimeric polypeptides that are relatively easy to be isolated and/or purified.

### Peptide Tags

In some embodiments, a single-chain chimeric polypeptide includes a peptide tag (e.g., at the N-terminal end or the C-terminal end of the single-chain chimeric polypeptide). In some embodiments, a single-chain chimeric polypeptide includes two or more peptide tags.

Exemplary peptide tags that can be included in a single-chain chimeric polypeptide include, without limitation, AviTag (GLNDIFEAQKIEWHE; SEQ ID NO: 65), a calmodulin-tag (KRRWKKNFIAVSAANRFKKISSSGAL; SEQ ID NO: 66), a polyglutamate tag (EEEEEE; SEQ ID NO: 67), an E-tag (GAPVPYPDPLEPR; SEQ ID NO: 68), a FLAG-tag (DYKDDDDK; SEQ ID NO: 69), an HA-tag, a peptide from hemagglutinin (YPYDVPDYA; SEQ ID NO: 70), a his-tag (HHHHH (SEQ ID NO: 71); HHHHHH (SEQ ID NO: 72); HHHHHHH (SEQ ID NO: 73); HHHHHHHH (SEQ ID NO: 74); HHHHHHHHH (SEQ ID NO: 75); or HHHHHHHHHH (SEQ ID NO: 76)), a myc-tag (EQKLISEEDL; SEQ ID NO: 77), NE-tag (TKENPRSNQEESYDDNES; SEQ ID NO: 78), S-tag, (KETAAAKFERQHMDS; SEQ ID NO: 79), SBP-tag (MDEKTTGWRGGHVVEGLAGELEQLRARLEHHPQGQREP; SEQ ID NO: 80), Softag 1 (SLAELLNAGLGGS; SEQ ID NO: 81), Softag 3 (TQDPSRVG; SEQ ID NO: 82), Spot-tag (PDRVRAVSHWSS; SEQ ID NO: 83), Strep-tag (WSHPQFEK; SEQ ID NO: 84), TC tag (CCPGCC; SEQ ID NO: 85), Ty tag (EVHTNQDPLD; SEQ ID NO: 86), V5 tag (GKPIPNPLLGLDST; SEQ ID NO: 87), VSV-tag (YTDIEMNRLGK; SEQ ID NO: 88), and Xpress tag (DLYDDDDK; SEQ ID NO: 89). In some embodiments, the peptide tag is a tissue factor protein. In some embodiments, the peptide tag is a TRβC domain.

Peptide tags that can be included in a single-chain chimeric polypeptide can be used in any of a variety of applications related to the single-chain chimeric polypeptide. For example, a peptide tag can be used in the purification of a single-chain chimeric polypeptide. As one non-limiting example, a single-chain chimeric polypeptide can include a myc tag; and can be purified using an antibody that recognizes the myc tag(s). One non-limiting example of an antibody that recognizes a myc tag is 9E10, available from the non-commercial Developmental Studies Hybridoma Bank. As another non-limiting example, a single-chain chimeric polypeptide can include a histidine tag and can be purified using a nickel or cobalt chelate. Those of ordinary skill in the art will be aware of other suitable tags and agent that bind those tags for use in purifying a single-chain chimeric polypeptide. In some embodiments, a peptide tag is removed from the single-chain chimeric polypeptide after purification. In some embodiments, a peptide tag is not removed from the single-chain chimeric polypeptide after purification.

In some embodiments, one or both of the first target-binding domain and the second target-binding domain can be used in the purification of a single-chain chimeric polypeptide. In some embodiments, wherein one or both of the first target-binding domain and the second target-binding domain is a tissue factor protein, the first target-binding domain and/or the second target-binding domain is used in the purification of a single-chain chimeric polypeptide. In some embodiments, the TRβC domain is used in the purification of a single-chain chimeric polypeptide. For example, a single-chain chimeric polypeptide can be purified using an antibody that recognizes the TRβC domain.

Peptide tags that can be included in a single-chain chimeric polypeptide can be used, for example, in immunoprecipitation of the single-chain chimeric polypeptide, imaging of the single-chain chimeric polypeptide (e.g., via Western blotting, ELISA, flow cytometry, and/or immunocytochemistry), and/or solubilization of the single-chain chimeric polypeptide.

In some embodiments, a single-chain chimeric polypeptide includes a peptide tag that is about 10 to 200 amino acids in length. For example, a peptide tag can be about 10 to 200 amino acids in length, about 15 to 200 amino acids in length, about 20 to 200 amino acids in length, about 25 to 200 amino acids in length, about 30 to 200 amino acids in length, about 35 to 200 amino acids in length, about 40 to 200 amino acids in length, about 45 to 200 amino acids in length, about 50 to 200 amino acids in length, about 55 to 200 amino acids in length, about 60 to 200 amino acids in length, about 65 to 200 amino acids in length, about 70 to 200 amino acids in length, about 75 to 200 amino acids in length, about 80 to 200 amino acids in length, about 85 to 200 amino acids in length, about 90 to 200 amino acids in length, about 95 to 200 amino acids in length, about 100 to 200 amino acids in length, about 110 to 200 amino acids in length, about 120 to 200 amino acids in length, about 130 to 200 amino acids in length, about 140 to 200 amino acids in length, about 150 to 200 amino acids in length, about 160 to 200 amino acids in length, about 170 to 200 amino acids in length, about 180 to 200 amino acids in length, about 190 to 200 amino acids in length, about 10 to 190 amino acids in length, about 15 to 190 amino acids in length, about 20 to 190 amino acids in length, about 25 to 190 amino acids in length, about 30 to 190 amino acids in length, about 35 to 190 amino acids in length, about 40 to 190 amino acids in length, about 45 to 190 amino acids in length, about 50 to 190 amino acids in length, about 55 to 190 amino acids in length, about 60 to 190 amino acids in length, about 65 to 190 amino acids in length, about 70 to 190 amino acids in length, about 75 to 190 amino acids in length, about 80 to 190 amino acids in length, about 85 to 190 amino acids in length, about 90 to 190 amino acids in length, about 95 to 190 amino acids in length, about 100 to 190 amino acids in length, about 110 to 190 amino acids in length, about 120 to 190 amino acids in length, about 130 to 190 amino acids in length, about 140 to 190 amino acids in length, about 150 to 190 amino acids in length, about 160 to 190 amino acids in length, about 170 to 190 amino acids in length, about 180 to 190 amino acids in length, about 10 to 180 amino acids in length, about 15 to 180 amino acids in length, about 20 to 180 amino acids in length, about 25 to 180 amino acids in length, about 30 to 180 amino acids in length, about 35 to 180 amino acids in length, about 40 to 180 amino acids in length, about 45 to 180 amino acids in length, about 50 to 180 amino acids in length, about 55 to 180 amino acids in length, about 60 to 180 amino acids in length, about 65 to 180 amino acids in length, about 70 to 180 amino acids in length, about 75 to 180 amino acids in length, about 80 to 180 amino acids in length, about 85 to 180 amino acids in length, about 90 to 180 amino acids in length, about 95 to 180 amino acids in length, about 100 to 180 amino acids in length, about 110 to 180 amino acids in length, about 120 to 180 amino acids in length, about 130 to 180 amino acids in length, about 140 to 180 amino acids in length, about 150 to 180 amino acids in length, about 160 to 180 amino acids in length, about 170 to 180 amino acids in length, about 10 to 170 amino acids in length, about 15 to 170 amino acids in length, about 20 to 170 amino acids in length, about 25 to 170 amino acids in length, about 30 to 170 amino acids in length, about 35 to 170 amino acids in length, about 40 to 170 amino acids in length, about 45 to 170 amino acids in length, about 50 to 170 amino acids in length, about 55 to 170 amino acids in length, about 60 to 170 amino acids in length, about 65 to 170 amino acids in length, about 70 to 170 amino acids in length, about 75 to 170 amino acids in length, about 80 to 170 amino acids in length, about 85 to 170 amino acids in length, about 90 to 170 amino acids in length, about 95 to 170 amino acids in length, about 100 to 170 amino acids in length, about 110 to 170 amino acids in length, about 120 to 170 amino acids in length, about 130 to 170 amino acids in length, about 140 to 170 amino acids in length, about 150 to 170 amino acids in length, about 160 to 170 amino acids in length, about 10 to 160 amino acids in length, about 15 to 160 amino acids in length, about 20 to 160 amino acids in length, about 25 to 160 amino acids in length, about 30 to 160 amino acids in length, about 35 to 160 amino acids in length, about 40 to 160 amino acids in length, about 45 to 160 amino acids in length, about 50 to 160 amino acids in length, about 55 to 160 amino acids in length, about 60 to 160 amino acids in length, about 65 to 160 amino acids in length, about 70 to 160 amino acids in length, about 75 to 160 amino acids in length, about 80 to 160 amino acids in length, about 85 to 160 amino acids in length, about 90 to 160 amino acids in length, about 95 to 160 amino acids in length, about 100 to 160 amino acids in length, about 110 to 160 amino acids in length, about 120 to 160 amino acids in length, about 130 to 160 amino acids in length, about 140 to 160 amino acids in length, about 150 to 160 amino acids in length, about 10 to 150 amino acids in length, about 15 to 150 amino acids in length, about 20 to 150 amino acids in length, about 25 to 150 amino acids in length, about 30 to 150 amino acids in length, about 35 to 150 amino acids in length, about 40 to 150 amino acids in length, about 45 to 150 amino acids in length, about 50 to 150 amino acids in length, about 55 to 150 amino acids in length, about 60 to 150 amino acids in length, about 65 to 150 amino acids in length, about 70 to 150 amino acids in length, about 75 to 150 amino acids in length, about 80 to 150 amino acids in length, about 85 to 150 amino acids in length, about 90 to 150 amino acids in length, about 95 to 150 amino acids in length, about 100 to 150 amino acids in length, about 110 to 150 amino acids in length, about 120 to 150 amino acids in length, about 130 to 150 amino acids in length, about 140 to 150 amino acids in length, about 10 to 140 amino acids in length, about 15 to 140 amino acids in length, about 20 to 140 amino acids in length, about 25 to 140 amino acids in length, about 30 to 140 amino acids in length, about 35 to 140 amino acids in length, about 40 to 140 amino acids in length, about 45 to 140 amino acids in length, about 50 to 140 amino acids in length, about 55 to 140 amino acids in length, about 60 to 140 amino acids in length, about 65 to 140 amino acids in length, about 70 to 140 amino acids in length, about 75 to 140 amino acids in length, about 80 to 140 amino acids in length, about 85 to 140 amino acids in length, about 90 to 140 amino acids in length, about 95 to 140 amino acids in length, about 100 to 140 amino acids in length, about 110 to 140 amino acids in length, about 120 to 140 amino acids in length, about 130 to 140 amino acids in length, about 10 to 130 amino acids in length, about 15 to 130 amino acids in length, about 20 to 130 amino acids in length, about 25 to 130 amino acids in length, about 30 to 130 amino acids in length, about 35 to 130 amino acids in length, about 40 to 130 amino acids in length, about 45 to 130 amino acids in length, about 50 to 130 amino acids in length, about 55 to 130 amino acids in length, about 60 to 130 amino acids in length, about 65 to 130 amino acids in length, about 70 to 130 amino acids in length, about 75 to 130 amino acids in length, about 80 to 130 amino acids in length, about 85 to 130 amino acids in length, about 90 to 130 amino acids in length, about 95 to 130 amino acids in length, about 100 to 130 amino acids in length, about 110 to 130 amino acids in length, about 120 to 130 amino acids in length, about 10 to 120 amino acids in length, about 15 to 120 amino acids in length, about 20 to 120 amino acids in length, about 25 to 120 amino acids in length, about 30 to 120 amino acids in length, about 35 to 120 amino acids in length, about 40 to 120 amino acids in length, about 45 to 120 amino acids in length, about 50 to 120 amino acids in length, about 55 to 120 amino acids in length, about 60 to 120 amino acids in length, about 65 to 120 amino acids in length, about 70 to 120 amino acids in length, about 75 to 120 amino acids in length, about 80 to 120 amino acids in length, about 85 to 120 amino acids in length, about 90 to 120 amino acids in length, about 95 to 120 amino acids in length, about 100 to 120 amino acids in length, about 110 to 120 amino acids in length, bout 10 to 110 amino acids in length, about 15 to 110 amino acids in length, about 20 to 110 amino acids in length, about 25 to 110 amino acids in length, about 30 to 110 amino acids in length, about 35 to 110 amino acids in length, about 40 to 110 amino acids in length, about 45 to 110 amino acids in length, about 50 to 110 amino acids in length, about 55 to 110 amino acids in length, about 60 to 110 amino acids in length, about 65 to 110 amino acids in length, about 70 to 110 amino acids in length, about 75 to 110 amino acids in length, about 80 to 110 amino acids in length, about 85 to 110 amino acids in length, about 90 to 110 amino acids in length, about 95 to 110 amino acids in length, about 100 to 110 amino acids in length, about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a peptide tag is about 10, 15, 20, 25, 30, 35, 40, 45, 50, 55, 60, 65, 70, 75, 80, 85, 90, 95, or 100 amino acids in length.

Peptide tags included in a single-chain chimeric polypeptide can be of any suitable length. For example, peptide tags can be 5, 6, 7, 8, 9, 10, 11, 12, 13, 14, 15, 16, 17, 18, 19, 20, or more amino acids in length. In embodiments in which a single-chain chimeric polypeptide includes two or more peptide tags, the two or more peptide tags can be of the same or different lengths. In some embodiments, any of the peptide tags disclosed herein may include one or more additional amino acids (e.g., 1, 2, 3, 5, 6, 7, 8, 9, 10, or more amino acids) at the N-terminus and/or C-terminus, so long as the function of the peptide tag remains intact. For example, a myc tag having the amino acid sequence EQKLISEEDL (SEQ ID NO: 77) can include one or more additional amino acids (e.g., at the N-terminus and/or the C-terminus of the peptide tag), while still retaining the ability to be bound by an antibody (e.g., 9E10).

### Exemplary Embodiments of Single-Chain Chimeric Polypeptides—Type A

In some embodiments of any of the single-chain chimeric polypeptides described herein, the first target-binding domain and/or the second target-binding domain can independently bind specifically to an IL-7 receptor (e.g., human IL-7 receptor) or a ligand of TGF-βRII (e.g., a ligand of human TGFβRII receptor). In some embodiments, the first target-binding domain binds specifically to an IL-7 receptor (e.g., human IL-7 receptor) and the second target-binding domain binds specifically to a ligand of TGF-βRII (e.g., a ligand of human TGFβRII receptor). In some embodiments, the first target-binding domain binds specifically to a ligand of TGF-βRII (e.g., a ligand of human TGFβRII receptor) and the second target-binding domain binds specifically to an IL-7 receptor (e.g., human IL-7 receptor).

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, the first target-binding domain is a soluble human IL-7 protein and the second target-binding domain is a soluble TGFβRII receptor. In some embodiments of these single-chain chimeric polypeptides, the first target-binding domain is a soluble TGFβRII receptor and the second target-binding domain is a soluble IL-7.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

In some embodiments, the ligand of TGF-βRII is a soluble TGFβRII receptor. In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides, the linker includes the sequence GGGGSGGGGSGGGGS (SEQ ID NO: 53).

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments of these single-chain chimeric polypeptides, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-tissue factor antigen-binding domain or an anti-PD-1 antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

### IL-7/TRβC/TGFβRII

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a soluble TGFβRII receptor (e.g., a sequence that is at least 80%, at least 90%, at least 95%, or 100% identical to SEQ ID NO: 9 or SEQ ID NO: 39).

In some embodiments, the single-chain chimeric polypeptide further includes an additional target-binding domain.

In some embodiments, the additional target-binding domain includes an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes an anti-TF antigen-binding domain. In some embodiments, the anti-TF antigen-binding domain includes an anti-human TF diabody (e.g., a sequence that is at least 80%, at least 90%, at least 95%, or 100% identical to SEQ ID NO: 99). In some embodiments, the anti-TF antigen-binding domain includes an anti-human TFscFv (e.g., a sequence that is at least 80%, at least 90%, at least 95%, or 100% identical to SEQ ID NO: 103).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a soluble TGFβRII receptor (e.g., a sequence that is at least 80%, at least 90%, at least 95%, or 100% identical to SEQ ID NO: 9 or SEQ ID NO: 39), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-human PD-1 diabody (e.g., a sequence that is at least 80%, at least 90%, at least 95%, or 100% identical to SEQ ID NO: 108), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-mouse PD-1 diabody (e.g., a sequence that is at least 80%, at least 90%, at least 95%, or 100% identical to SEQ ID NO: 110), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-PD-1 scFv (e.g., a sequence that is at least 80%, at least 90%, at least 95%, or 100% identical to SEQ ID NO: 111), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type B

In some embodiments of any of the single-chain chimeric polypeptides described herein, the first target-binding domain and/or the second target-binding domain can independently bind specifically to an IL-7 receptor (e.g., human IL-7 receptor) or an IL-21 receptor (e.g., human IL-21 receptor). In some embodiments of any of the single-chain chimeric polypeptides described herein, the first target-binding domain binds specifically to an IL-7 receptor (e.g., human IL-7 receptor) and the second target-binding domain binds specifically to an IL-21 receptor (e.g., human IL-21 receptor). In some embodiments of any of the single-chain chimeric polypeptides described herein, the first target-binding domain binds specifically to an IL-21 receptor (e.g., human IL-21 receptor) and the second target-binding domain binds specifically to an IL-7 receptor (e.g., human IL-7 receptor).

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments of these single-chain chimeric polypeptides, the soluble TRβC domain can be any of the exemplary soluble TRβC domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, the first target-binding domain is a soluble IL-7 and the second target-binding domain is a soluble human IL-21.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 5.

In some embodiments, an IL-21 that binds specifically to an IL-21 receptor can be encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 6 or SEQ ID NO: 11.

In some embodiments of these single-chain chimeric polypeptides, the first target-binding domain is a soluble IL-7 and the second target-binding domain is a soluble mouse IL-21.

In some embodiments, an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 132.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-tissue factor antigen-binding domain or an anti-PD-1 antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

### IL-7/TRβC/IL-21

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes a soluble IL-21 (e.g., a sequence that is at least 80%, at least 90%, at least 95%, or 100% identical to SEQ ID NO: 5 or SEQ ID NO: 132).

In some embodiments, the single-chain chimeric polypeptide further includes an additional target-binding domain.

In some embodiments, the additional target-binding domain includes or is an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes or is an anti-TF antigen-binding domain. In some embodiments, the anti-TF antigen-binding domain includes an anti-human TF diabody (e.g., a sequence that is at least 80%, at least 90%, at least 95%, or 100% identical to SEQ ID NO: 99). In some embodiments, the anti-TF antigen-binding domain includes an anti-human TFscFv (e.g., a sequence that is at least 80%, at least 90%, at least 95%, or 100% identical to SEQ ID NO: 103).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a human IL-21 (e.g., a sequence that is at least 80%, at least 90%, at least 95%, or 100% identical to SEQ ID NO: 5), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a mouse IL-21 (e.g., a sequence that is at least 80%, at least 90%, at least 95%, or 100% identical to SEQ ID NO: 132), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a human IL-21 (e.g., a sequence that is at least 80%, at least 90%, at least 95%, or 100% identical to SEQ ID NO: 5), and (iv) an additional target-binding domain that includes an anti-TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a mouse IL-21 (e.g., a sequence that is at least 80%, at least 90%, at least 95%, or 100% identical to SEQ ID NO: 132), and (iv) an additional target-binding domain that includes an anti-TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a human IL-21 (e.g., a sequence that is at least 80%, at least 90%, at least 95%, or 100% identical to SEQ ID NO: 5), and (iv) an additional target-binding domain that includes an anti-PD-1 scFv (e.g., a sequence that is at least 80%, at least 90%, at least 95%, or 100% identical to SEQ ID NO: 111 or SEQ ID NO: 112), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a mouse IL-21 (e.g., a sequence that is at least 80%, at least 90%, at least 95%, or 100% identical to SEQ ID NO: 132), and (iv) an additional target-binding domain that includes an anti-PD-1 scFv (e.g., a sequence that is at least 80%, at least 90%, at least 95%, or 100% identical to SEQ ID NO: 111 or SEQ ID NO: 112), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a human IL-21 (e.g., a sequence that is at least 80%, at least 90%, at least 95%, or 100% identical to SEQ ID NO: 5), and (iv) an additional target-binding domain that includes an anti-PD-1 diabody (e.g., a sequence that is at least 80%, at least 90%, at least 95%, or 100% identical to SEQ ID NO: 108 or SEQ ID NO: 110), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a mouse IL-21 (e.g., a sequence that is at least 80%, at least 90%, at least 95%, or 100% identical to SEQ ID NO: 132), and (iv) an additional target-binding domain that includes an anti-PD-1 diabody (e.g., a sequence that is at least 80%, at least 90%, at least 95%, or 100% identical to SEQ ID NO: 108 or SEQ ID NO: 110), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type C

In some embodiments of any of the single-chain chimeric polypeptides described herein, the first target-binding domain binds specifically to a ligand of TGF-βRII and the second target-binding domain binds specifically to a ligand of TGF-βRII.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, the first target-binding domain is a soluble TGFβRII receptor and the second target-binding domain is a soluble TGFβRII receptor.

In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides, the linker includes the sequence GGGGSGGGGSGGGGS (SEQ ID NO: 53).

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments of these single-chain chimeric polypeptides, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-tissue factor antigen-binding domain, an anti-PD-1 antigen-binding domain, or a soluble IL-7.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

In some embodiments, a soluble IL-7 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 4.

### TGFβRII/TRβC/TGFβRII

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes a soluble TGFβRII receptor (e.g., a sequence that is at least 80%, at least 90%, at least 95%, or 100% identical to SEQ ID NO: 9 or SEQ ID NO: 39).

In some embodiments, the single-chain chimeric polypeptide further includes an additional target-binding domain.

In some embodiments, the additional target-binding domain includes an anti-PD-1 antigen-binding domain. In some embodiments, the anti-PD-1 antigen-binding domain includes or is an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes or is an anti-TF antigen-binding domain. In some embodiments, the anti-TF antigen-binding domain includes an anti-human TF diabody (e.g., a sequence that is at least 80%, at least 90%, at least 95%, or 100% identical to SEQ ID NO: 99). In some embodiments, the anti-TF antigen-binding domain includes an anti-human TFscFv (e.g., a sequence that is at least 80%, at least 90%, at least 95%, or 100% identical to SEQ ID NO: 103).

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes a soluble TGFβRII receptor (e.g., a sequence that is at least 80%, at least 90%, at least 95%, or 100% identical to SEQ ID NO: 9 or SEQ ID NO: 39), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-human TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-human PD-1 scFv (e.g., a sequence that is at least 80%, at least 90%, at least 95%, or 100% identical to SEQ ID NO: 111), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes an anti-mouse PD-1 scFv (e.g., a sequence that is at least 80%, at least 90%, at least 95%, or 100% identical to SEQ ID NO: 112), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes a soluble TGFβRII receptor (e.g., a sequence that is at least 80%, at least 90%, at least 95%, or 100% identical to SEQ ID NO: 9 or SEQ ID NO: 39), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type D

In some embodiments of any of the single-chain chimeric polypeptides described herein, the first target-binding domain and/or the second target-binding domain can each independently bind specifically to a receptor of IL-7, to a ligand of TGF-βRII, or to CD3.

In some embodiments, the first target-binding domain and the second target-binding domain both bind specifically to a receptor of IL-7 or to CD3. In some embodiments, the first target-binding domain binds specifically to a receptor for IL-7, and the second target-binding domain binds specifically to CD3. In some embodiments, where the second target-binding domain binds specifically to CD3, the second target-binding domain can further bind specifically to tissue factor (TF). In some embodiments, the first target-binding domain binds specifically to CD3, and the second target-binding domain bind specifically to a receptor for IL-7. In some embodiments, where the first target-binding domain binds specifically to CD3, the first target-binding domain can further bind specifically to tissue factor.

In some embodiments, the first target-binding domain and the second target-binding domain both bind specifically to a ligand of TGF-βRII or to CD3. In some embodiments, the first target-binding domain binds specifically to a ligand of TGF-βRII, and the second target-binding domain binds specifically to CD3. In some embodiments, where the second target-binding domain binds specifically to CD3, the second target-binding domain can further bind specifically to tissue factor (TF). In some embodiments, the first target-binding domain binds specifically to CD3, and the second target-binding domain bind specifically to a ligand of TGF-βRII. In some embodiments, where first second target-binding domain binds specifically to CD3, the first target-binding domain can further bind specifically to tissue factor.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments, one or both of the first target-binding domain and the second target-binding domain includes or is a soluble IL-7 or an anti-CD3 antigen-binding domain. In some embodiments, the first target-binding domain includes or is a soluble human IL-7, and the second target-binding domain includes or is an anti-CD3 antigen-binding domain. In some embodiments, where the second target-binding domain includes an anti-CD3 antigen-binding domain, the second target-binding domain further includes an anti-tissue factor antigen-binding domain. In some embodiments, the first target-binding domain is anti-CD3 antigen-binding domain, and the second target-binding domain is a soluble human IL-7. In some embodiments, where the first target-binding domain includes an anti-CD3 antigen-binding domain, the first target-binding domain further includes an anti-tissue factor antigen-binding domain.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

In some embodiments, one or both of the first target-binding domain and the second target-binding domain includes or is a soluble TGFβRII receptor or an anti-CD3 antigen-binding domain. In some embodiments, the first target-binding domain includes or is a soluble TGFβRII receptor, and the second target-binding domain includes or is an anti-CD3 antigen-binding domain. In some embodiments, where the second target-binding domain includes an anti-CD3 antigen-binding domain, the second target-binding domain further includes an anti-tissue factor antigen-binding domain. In some embodiments, the first target-binding domain is anti-CD3 antigen-binding domain, and the second target-binding domain is a soluble TGFβRII receptor. In some embodiments, where the first target-binding domain includes an anti-CD3 antigen-binding domain, the first target-binding domain further includes an anti-tissue factor antigen-binding domain.

In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides.

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments of these single-chain chimeric polypeptides, the anti-CD3 antigen-binding domain includes or is an anti-CD3 scFv. A non-limiting example of an anti-CD3 scFv includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 93.

In some embodiments, an anti-CD3 scFv can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 94.

In some embodiments, an anti-CD3 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 95.

In some embodiments, an anti-CD3 scFv is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 96.

In some embodiments, an anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 41 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 42.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 45.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 97.

In some embodiments, the anti-CD3 antigen-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 137.

In some embodiments, a first target-binding domain and/or a second target-binding domain that includes both an anti-tissue factor domain and an anti-CD3 binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 105.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

### TGFβRII/TRβC/Anti-CD3scFv

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the single-chain chimeric polypeptide further includes an additional target-binding domain.

In some embodiments, the additional target-binding domain includes or is an anti-TF antigen-binding domain. In some embodiments, the anti-TF antigen-binding domain is an anti-human TF diabody (e.g., a sequence that is at least 80%, at least 90%, at least 95%, or 100% identical to SEQ ID NO: 99). In some embodiments, the anti-TF antigen-binding domain is an anti-human TFscFv (e.g., a sequence that is at least 80%, at least 90%, at least 95%, or 100% identical to SEQ ID NO: 103).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-CD3scFv (e.g., a sequence that is at least 80%, at least 90%, at least 95%, or 100% identical to any one of SEQ ID NO: 93, SEQ ID NO: 95, and SEQ ID NO: 97), and (iv) an additional target-binding domain that includes an anti-TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

### IL-7/TRβC/Anti-CD3scFv

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the single-chain chimeric polypeptide further includes an additional target-binding domain.

In some embodiments, the additional target-binding domain includes or is an anti-TF antigen-binding domain. In some embodiments, the anti-TF antigen-binding domain is an anti-human TF diabody (e.g., a sequence that is at least 80%, at least 90%, at least 95%, or 100% identical to SEQ ID NO: 99). In some embodiments, the anti-TF antigen-binding domain is an anti-human TFscFv (e.g., a sequence that is at least 80%, at least 90%, at least 95%, or 100% identical to SEQ ID NO: 103).

In some embodiments, the single-chain chimeric polypeptide further includes a signal sequence at its N-terminal end. In some embodiments the signal peptide sequence is MKWVTFISLLFLFSSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-CD3scFv (e.g., a sequence that is at least 80%, at least 90%, at least 95%, or 100% identical to any one of SEQ ID NO: 93, SEQ ID NO: 95, and SEQ ID NO: 97), and (iv) an additional target-binding domain that includes an anti-TF diabody (e.g., a sequence that is at least 80%, at least 90%, at least 95%, or 100% identical to SEQ ID NO: 99), wherein the first target-binding domain, the soluble T cell receptor β-chain constant region (TRβC) domain, the second target-binding domain, and the additional target-binding domain are positioned in a N- to C-terminal direction.

### IL-7/TRβC/Anti-TFscFv (Humanized)-Anti-CD3scFv

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes a humanized anti-human TFscFv and an anti-human CD3scFv (e.g., a sequence that is at least 80%, at least 90%, at least 95%, or 100% identical to SEQ ID NO: 105).

In some embodiments, the single-chain chimeric polypeptide further includes a signal sequence at its N-terminal end. In some embodiments the signal peptide sequence is MKWVTFISLLFLFSSAYS (SEQ ID NO: 57). In some embodiments the signal peptide sequence is MGVKVLFALICIAVAEA (SEQ ID NO: 129).

### TGFβRII/TRβC/Anti-TFscFv (Humanized)-Anti-CD3scFv

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes a humanized anti-human TFscFv and an anti-human CD3scFv (e.g., a sequence that is at least 80%, at least 90%, at least 95%, or 100% identical to SEQ ID NO: 105).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

### Exemplary Embodiments of Single-Chain Chimeric Polypeptides—Type E

In some embodiments of any of the single-chain chimeric polypeptides described herein, the first target-binding domain and/or the second target-binding domain can independently bind specifically to a receptor of IL-7, to a ligand of TGF-βRII, or to tissue factor.

In some embodiments of these single-chain chimeric polypeptides, the first target-binding domain and the second target-binding domain both bind specifically to a receptor of IL-7 or to tissue factor. In some embodiments, the first target-binding domain binds specifically to a receptor for IL-7, and the second target-binding domain binds specifically to tissue factor. In some embodiments, the first target-binding domain binds specifically to tissue factor, and the second target-binding domain bind specifically to a receptor for IL-7.

In some embodiments of these single-chain chimeric polypeptides, the first target-binding domain and the second target-binding domain both bind specifically to a ligand of TGF-βRII or to tissue factor. In some embodiments, the first target-binding domain binds specifically to a ligand of TGF-βRII, and the second target-binding domain binds specifically to tissue factor. In some embodiments, the first target-binding domain binds specifically to tissue factor, and the second target-binding domain bind specifically to a ligand of TGF-βRII.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, one or both of the first target-binding domain and the second target-binding domain is a soluble IL-7 or an anti-tissue factor antigen-binding domain. In some embodiments, the first target-binding domain and the second target-binding domain are each independently a soluble IL-7 or an anti-tissue factor antigen-binding domain. In some embodiments, the first target-binding domain is a soluble IL-7, and the second target-binding domain is an anti-tissue factor antigen-binding domain. In some embodiments of these single-chain chimeric polypeptides, the first target-binding domain is an anti-tissue factor antigen-binding domain, and the second target-binding domain is a soluble IL-7.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

In some embodiments of these single-chain chimeric polypeptides, one or both of the first target-binding domain and the second target-binding domain is a soluble TGFβRII receptor or an anti-tissue factor antigen-binding domain. In some embodiments, the first target-binding domain and the second target-binding domain are each independently a soluble TGFβRII receptor or an anti-tissue factor antigen-binding domain. In some embodiments, the first target-binding domain is a soluble TGFβRII receptor, and the second target-binding domain is an anti-tissue factor antigen-binding domain. In some embodiments, the first target-binding domain is an anti-tissue factor antigen-binding domain, and the second target-binding domain is a soluble TGFβRII receptor.

In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides, the linker includes the sequence GGGGSGGGGSGGGGS (SEQ ID NO: 53).

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-CD3 antigen-binding domain, an anti-PD-1 antigen-binding domain, an anti-CD16a antigen-binding domain, or a soluble IL-21.

In some embodiments of these single-chain chimeric polypeptides, the anti-CD3 antigen-binding domain includes or is an anti-CD3 scFv. A non-limiting example of an anti-CD3 scFv includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 93.

In some embodiments, an anti-CD3 scFv can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 94.

In some embodiments, an anti-CD3 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 95.

In some embodiments, an anti-CD3 scFv is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 96.

In some embodiments, an anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 41 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 42.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 45.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 97.

In some embodiments, the anti-CD3 antigen-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 137.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

In some embodiments, an anti-CD16a antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 125.

In some embodiments, a soluble IL-21 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 5 or SEQ ID NO: 132.

### IL-7/TRβC/Anti-TFscFv

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human TFscFv (e.g., a sequence that is at least 80%, at least 90%, at least 95%, or 100% identical to SEQ ID NO: 103).

In some embodiments, the single-chain chimeric polypeptide further includes an additional target-binding domain.

In some embodiments, the additional target-binding domain includes or is an anti-CD3 antigen-binding domain. In some embodiments, the anti-CD3 antigen-binding domain includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the additional target-binding domain includes or is an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes or is an anti-CD16a antigen-binding domain. In some embodiments, the anti-CD16a antigen-binding domain includes an anti-CD16a scFv (e.g., a sequence that is at least 80%, at least 90%, at least 95%, or 100% identical to any one of SEQ ID NO: 125).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 93), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 95), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 97), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-human PD-1 scFv (e.g., a sequence that is at least 80%, at least 90%, at least 95%, or 100% identical to SEQ ID NO: 111), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-mouse PD-1 scFv (e.g., a sequence that is at least 80%, at least 90%, at least 95%, or 100% identical to SEQ ID NO: 112), wherein the first target-binding domain, the soluble T cell receptor β-chain constant region (TRβC) domain, the second target-binding domain, and the additional target-binding domain are positioned in a N- to C-terminal direction.

### IL-7/TRβC/Anti-TF (Diabody)

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-human TF diabody (e.g., a sequence that is at least 80%, at least 90%, at least 95%, or 100% identical to SEQ ID NO: 99).

In some embodiments, the single-chain chimeric polypeptide further includes an additional target-binding domain.

In some embodiments, the additional target-binding domain includes or is an anti-CD3 antigen-binding domain. In some embodiments, the anti-CD3 antigen-binding domain includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the additional target-binding domain includes or is an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes or is an anti-CD16a antigen-binding domain. In some embodiments, the anti-CD16a antigen-binding domain includes an anti-CD16a scFv (e.g., a sequence that is at least 80%, at least 90%, at least 95%, or 100% identical to any one of SEQ ID NO: 125).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 93), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 95), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 97), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes a human IL-21 (e.g., a sequence that is at least 80%, at least 90%, at least 95%, or 100% identical to SEQ ID NO: 5), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes a mouse IL-21 (e.g., a sequence that is at least 80%, at least 90%, at least 95%, or 100% identical to SEQ ID NO: 132), wherein the first target-binding domain, the soluble T cell receptor β-chain constant region (TRβC) domain, the second target-binding domain, and the additional target-binding domain are positioned in a N- to C-terminal direction.

### TGFβRII/TRβC/Anti-TF (Diabody)

In some embodiments, provided herein is a single-chain chimeric polypeptide that includes (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-human TF diabody (e.g., a sequence that is at least 80%, at least 90%, at least 95%, or 100% identical to SEQ ID NO: 99).

In some embodiments, the single-chain chimeric polypeptide further includes an additional target-binding domain.

In some embodiments, the additional target-binding domain includes or is an anti-CD3 antigen-binding domain. In some embodiments, the anti-CD3 antigen-binding domain includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the additional target-binding domain includes or is an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-PD-1 scFv (e.g., a sequence that is at least 80%, at least 90%, at least 95%, or 100% identical to SEQ ID NO: 111 or SEQ ID NO: 112).

In some embodiments, the additional target-binding domain includes or is an anti-CD16a antigen-binding domain. In some embodiments, the anti-CD16a antigen-binding domain includes an anti-CD16a scFv (e.g., a sequence that is at least 80%, at least 90%, at least 95%, or 100% identical to any one of SEQ ID NO: 125).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 93), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 95), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); (iii) a second target-binding domain that includes an anti-human TF diabody (e.g., a sequence that is at least 80%, at least 90%, at least 95%, or 100% identical to SEQ ID NO: 99), and (iv) an additional target-binding domain that includes an anti-CD3 scFv (e.g., a sequence that is at least 80%, at least 90%, at least 95%, or 100% identical to SEQ ID NO: 97), wherein the first target-binding domain, the soluble T cell receptor β-chain constant region (TRβC) domain, the second target-binding domain, and the additional target-binding domain are positioned in a N- to C-terminal direction.

### TGFβRII/TRβC/Anti-TFscFv

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-human TFscFv (e.g., a sequence that is at least 80%, at least 90%, at least 95%, or 100% identical to SEQ ID NO: 103).

In some embodiments, the single-chain chimeric polypeptide further includes an additional target-binding domain.

In some embodiments, the additional target-binding domain includes or is an anti-CD3 antigen-binding domain. In some embodiments, the anti-CD3 antigen-binding domain includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the additional target-binding domain includes or is an anti-PD-1 antigen-binding domain. In some embodiments, the anti-PD-1 antigen-binding domain includes an anti-human PD-1 diabody (e.g., a sequence that is at least 80%, at least 90%, at least 95%, or 100% identical to SEQ ID NO: 108). In some embodiments, the anti-PD-1 antigen-binding domain includes an anti-mouse PD-1 diabody (e.g., a sequence that is at least 80%, at least 90%, at least 95%, or 100% identical to SEQ ID NO: 110). In some embodiments, the anti-PD-1 antigen-binding domain includes an anti-human PD-1 scFv (e.g., a sequence that is at least 80%, at least 90%, at least 95%, or 100% identical to SEQ ID NO: 111). In some embodiments, the anti-PD-1 antigen-binding domain includes an anti-mouse PD-1 scFv (e.g., a sequence that is at least 80%, at least 90%, at least 95%, or 100% identical to SEQ ID NO: 112).

In some embodiments, the additional target-binding domain includes or is an anti-CD16a antigen-binding domain. In some embodiments, the anti-CD16a antigen-binding domain includes an anti-CD16a scFv (e.g., a sequence that is at least 80%, at least 90%, at least 95%, or 100% identical to any one of SEQ ID NO: 125).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 93), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 95), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD3 scFv (e.g., a sequence that is at least 80%, at least 90%, at least 95%, or 100% identical to SEQ ID NO: 97), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-human PD-1 scFv (e.g., a sequence that is at least 80%, at least 90%, at least 95%, or 100% identical to SEQ ID NO: 111), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-mouse PD-1 scFv (e.g., a sequence that is at least 80%, at least 90%, at least 95%, or 100% identical to SEQ ID NO: 112), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human TFscFv (e.g., a sequence that is at least 80%, at least 90%, at least 95%, or 100% identical to SEQ ID NO: 103), and (iv) an additional target-binding domain that includes an anti-CD16a scFv (e.g., a sequence that is at least 80%, at least 90%, at least 95%, or 100% identical to SEQ ID NO: 125), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type F

In some embodiments of the single-chain chimeric polypeptides described herein, the first target-binding domain and/or the second target-binding domain can each independently bind specifically to an IL-7 receptor, to PD-1, or to an IL-4 receptor. In some embodiments, the first target-binding domain and the second target-binding domain both bind specifically to an IL-7 receptor, to PD-1, or to an IL-4 receptor.

In some embodiments, the first target-binding domain binds specifically to an IL-7 receptor, and the second target-binding domain binds specifically to an IL-4 receptor. In some embodiments, the first target-binding domain binds specifically to an IL-4 receptor, and the second target-binding domain binds specifically to an IL-7 receptor.

In some embodiments, the first target-binding domain binds specifically to an IL-7 receptor, and the second target-binding domain binds specifically to PD-1. In some embodiments, the first target-binding domain binds specifically to PD-1, and the second target-binding domain bind specifically to an IL-7 receptor.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, one or both of the first target-binding domain and the second target-binding domain is a soluble IL-7 or a soluble IL-4. In some embodiments, the first target-binding domain and the second target-binding domain are each independently a soluble IL-7 or a soluble IL-4. In some embodiments, the first target-binding domain is a soluble IL-7 and the second target-binding domain is a soluble IL-4.

In some embodiments, the first target-binding domain is a soluble IL-7 and the second target-binding domain is a soluble human IL-4. A non-limiting example of an IL-4 that binds specifically to an IL-4 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 91.

In some embodiments, the first target-binding domain is a soluble IL-7 and the second target-binding domain is a soluble mouse IL-4. In some embodiments, a mouse soluble IL-4 can include a sequence that is at least 70% identical, at least 75% identical, at least 80% identical, at least 85% identical, at least 90% identical, at least 95% identical, at least 99% identical, or 100% identical to SEQ ID NO: 90.

In some embodiments of these single-chain chimeric polypeptides, one or both of the first target-binding domain and the second target-binding domain is a soluble IL-7 or an anti-PD-1 antigen-binding domain. In some embodiments of these single-chain chimeric polypeptides, the first target-binding domain and the second target-binding domain are each independently a soluble IL-7 or an anti-PD-1 antigen-binding domain. In some embodiments of these single-chain chimeric polypeptides, the first target-binding domain is a soluble IL-7 and the second target-binding domain is an anti-PD-1 antigen-binding domain. In some embodiments of these single-chain chimeric polypeptides, the first target-binding domain is a soluble IL-7 and the second target-binding domain is an anti-PD-1 antigen-binding domain.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is soluble IL-21. In some embodiments, a soluble IL-21 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 5 or SEQ ID NO: 132.

### IL-7/TRβC/IL-4

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an IL-4 (e.g., a sequence that is at least 80%, at least 90%, at least 95%, or 100% identical to SEQ ID NO: 90 or SEQ ID NO: 91).

In some embodiments, the single-chain chimeric polypeptide further includes an additional target-binding domain.

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a human IL-4 (e.g., a sequence that is at least 80%, at least 90%, at least 95%, or 100% identical to SEQ ID NO: 91), and (iv) an additional target-binding domain that includes a human IL-21 (e.g., a sequence that is at least 80%, at least 90%, at least 95%, or 100% identical to SEQ ID NO: 5), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a mouse IL-4 (e.g., a sequence that is at least 80%, at least 90%, at least 95%, or 100% identical to SEQ ID NO: 90), and (iv) an additional target-binding domain that includes a mouse IL-21 (e.g., a sequence that is at least 80%, at least 90%, at least 95%, or 100% identical to SEQ ID NO: 132), wherein the first target-binding domain, the soluble T cell receptor β-chain constant region (TRβC) domain, the second target-binding domain, and the additional target-binding domain are positioned in a N- to C-terminal direction.

### IL-7/TRβC/Anti-PD-1-scFv

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 scFv (e.g., a sequence that is at least 80%, at least 90%, at least 95%, or 100% identical to SEQ ID NO: 111 or SEQ ID NO: 112).

In some embodiments, the single-chain chimeric polypeptide further includes an additional target-binding domain.

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the single-chain chimeric polypeptide further includes a signal sequence at its N-terminal end. In some embodiments the signal peptide sequence is MKWVTFISLLFLFSSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human PD-1 scFv (e.g., a sequence that is at least 80%, at least 90%, at least 95%, or 100% identical to SEQ ID NO: 111), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human PD-1 scFv (e.g., a sequence that is at least 80%, at least 90%, at least 95%, or 100% identical to SEQ ID NO: 111), and (iv) an additional target-binding domain that includes a human IL-21 (e.g., a sequence that is at least 80%, at least 90%, at least 95%, or 100% identical to SEQ ID NO: 5), wherein the first target-binding domain, the soluble T cell receptor β-chain constant region (TRβC) domain, the second target-binding domain, and the additional target-binding domain are positioned in a N- to C-terminal direction.

### IL-7/TRβC/Anti-PD-1 (Diabody)

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-PD-1 diabody (e.g., a sequence that is at least 80%, at least 90%, at least 95%, or 100% identical to SEQ ID NO: 108 or SEQ ID NO: 110).

In some embodiments, the single-chain chimeric polypeptide further includes an additional target-binding domain.

In some embodiments, the additional target-binding domain includes or is a soluble IL-21 (e.g., a sequence that is at least 80%, at least 90%, at least 95%, or 100% identical to SEQ ID NO: 5 or SEQ ID NO: 132).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human PD-1 diabody (e.g., a sequence that is at least 80%, at least 90%, at least 95%, or 100% identical to SEQ ID NO: 108), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble IL-7 (e.g., a sequence that is at least 80%, at least 90%, at least 95%, or 100% identical to SEQ ID NO: 4), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human PD-1 diabody (e.g., a sequence that is at least 80%, at least 90%, at least 95%, or 100% identical to SEQ ID NO: 108), and (iv) an additional target-binding domain that includes a human IL-21 (e.g., a sequence that is at least 80%, at least 90%, at least 95%, or 100% identical to SEQ ID NO: 5), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type G

In some embodiments of the single-chain chimeric polypeptides described herein, the first target-binding domain and/or the second target-binding domain can each independently bind specifically to PD-1, to a ligand of TGF-βRII, or to tissue factor.

In some embodiments of single-chain chimeric polypeptides, the first target-binding domain and the second target-binding domain both bind specifically to PD-1 or to tissue factor. In some embodiments of these single-chain chimeric polypeptides, the first target-binding domain binds specifically to PD-1, and the second target-binding domain binds specifically to tissue factor. In some embodiments, the first target-binding domain binds specifically to tissue factor, and the second target-binding domain binds specifically to a PD-1.

In some embodiments, the first target-binding domain and the second target-binding domain both bind specifically to a ligand of TGF-βRII or to PD-1. In some embodiments, the first target-binding domain binds specifically to a ligand of TGF-βRII, and the second target-binding domain binds specifically to PD-1. In some embodiments, the first target-binding domain binds specifically to PD-1, and the second target-binding domain bind specifically to a ligand of TGF-βRII.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein. In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments, one or both of the first target-binding domain and the second target-binding domain is an anti-PD-1 antigen-binding domain or an anti-tissue factor antigen-binding domain. In some embodiments, the first target-binding domain and the second target-binding domain are each independently an anti-PD-1 antigen-binding domain or an anti-tissue factor antigen-binding domain. In some embodiments, the first target-binding domain is an anti-PD-1 antigen-binding domain, and the second target-binding domain is an anti-tissue factor antigen-binding domain. In some embodiments, the first target-binding domain is an anti-tissue factor antigen-binding domain, and the second target-binding domain is an anti-PD-1 antigen-binding domain.

In some embodiments, one or both of the first target-binding domain and the second target-binding domain is a soluble TGFβRII receptor or an anti-PD-1 antigen-binding domain. In some embodiments, the first target-binding domain and the second target-binding domain are each independently a soluble TGFβRII receptor or an anti-PD-1 antigen-binding domain. In some embodiments, the first target-binding domain is a soluble TGFβRII receptor, and the second target-binding domain is an anti-PD-1 antigen-binding domain. In some embodiments, the first target-binding domain is an anti-PD-1 antigen-binding domain, and the second target-binding domain is a soluble TGFβRII receptor.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 107, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 107.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 108.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 109.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 110.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 111.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 112.

In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides, the linker includes the sequence GGGGSGGGGSGGGGS (SEQ ID NO: 53).

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is soluble IL-7. In some embodiments, a soluble IL-7 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 4.

Anti-Human TFscFv//TRβC/Anti-PD-1 scFv

In some embodiments, provided herein is a sinngle-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 scFv (e.g., a sequence that is at least 80%, at least 90%, at least 95%, or 100% identical to SEQ ID NO: 111 or SEQ ID NO: 112).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLFSSAYS (SEQ ID NO: 57). In some embodiments the signal peptide sequence is MGVKVLFALICIAVAEA (SEQ ID NO: 129).

In some embodiments, provided herein is a sinngle-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-PD-1 scFv (e.g., a sequence that is at least 80%, at least 90%, at least 95%, or 100% identical to SEQ ID NO: 111 or SEQ ID NO: 112)), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

Anti-Human TF (Diabody)/TRβC/Anti-PD-1 scFv

In some embodiments, provided herein is a single-chain chimeric polypeptide that includes: (i) a first target-binding domain that includes an anti-human TF diabody (e.g., a sequence that is at least 80%, at least 90%, at least 95%, or 100% identical to SEQ ID NO: 99); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a second target-binding domain that includes an anti-PD-1 scFv (e.g., a sequence that is at least 80%, at least 90%, at least 95%, or 100% identical to SEQ ID NO: 111 or SEQ ID NO: 112).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

### TGFβRII/TRβC/Anti-PD-1 (Diabody)

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 diabody (e.g., a sequence that is at least 80%, at least 90%, at least 95%, or 100% identical to SEQ ID NO: 108 or SEQ ID NO: 110).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLFSSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human PD-1 diabody (e.g., a sequence that is at least 80%, at least 90%, at least 95%, or 100% identical to SEQ ID NO: 108), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human PD-1 diabody (e.g., a sequence that is at least 80%, at least 90%, at least 95%, or 100% identical to SEQ ID NO: 108), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-mouse PD-1 diabody (e.g., a sequence that is at least 80%, at least 90%, at least 95%, or 100% identical to SEQ ID NO: 110), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

TGFβRII/TRβC/Anti-PD-1 scFv

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 scFv (e.g., a sequence that is at least 80%, at least 90%, at least 95%, or 100% identical to SEQ ID NO: 111 or SEQ ID NO: 112).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

### Anti-Human TFscFv//TRβC/Anti-PD-1 (Diabody)

In some embodiments, provided herein is a single-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 diabody (e.g., a sequence that is at least 80%, at least 90%, at least 95%, or 100% identical to SEQ ID NO: 108 or SEQ ID NO: 110).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-human PD-1 diabody (e.g., a sequence that is at least 80%, at least 90%, at least 95%, or 100% identical to SEQ ID NO: 108), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-human PD-1 diabody (e.g., a sequence that is at least 80%, at least 90%, at least 95%, or 100% identical to SEQ ID NO: 108), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

In some embodiments, provided herein is a single-chain chimeric polypeptide that includes: (i) a first target-binding domain that includes an anti-human TFscFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes an anti-mouse PD-1 diabody (e.g., a sequence that is at least 80%, at least 90%, at least 95%, or 100% identical to SEQ ID NO: 110), and (iv) an additional target-binding domain that includes a soluble IL-7 (e.g., a sequence that is at least 80%, at least 90%, at least 95%, or 100% identical to SEQ ID NO: 4), wherein the first target-binding domain, the soluble T cell receptor β-chain constant region (TRβC) domain, the second target-binding domain, and the additional target-binding domain are positioned in a N- to C-terminal direction.

### Anti-TF (Diabody)/TRβC/Anti-PD-1 (Diabody)

In some embodiments, provided herein is a single-chain chimeric polypeptide that includes: (i) a first target-binding domain that includes an anti-human diabody (e.g., a sequence that is at least 80%, at least 90%, at least 95%, or 100% identical to SEQ ID NO: 99), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-PD-1 diabody (e.g., a sequence that is at least 80%, at least 90%, at least 95%, or 100% identical to SEQ ID NO: 108 or SEQ ID NO: 110).

In some embodiments, the single-chain chimeric polypeptide further includes an additional target-binding domain.

In some embodiments, the additional target-binding domain includes a soluble IL-7 (e.g., a sequence that is at least 80%, at least 90%, at least 95%, or 100% identical to SEQ ID NO: 4).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

### Exemplary Embodiments of Single-Chain Chimeric Polypeptides—Type H

In some embodiments of any of the single-chain chimeric polypeptides described herein, the first target-binding domain and/or the second target-binding domain can independently bind specifically to tissue factor or to CD16a. In some embodiments of these single-chain chimeric polypeptides, the first target-binding domain and the second target-binding domain both bind specifically to tissue factor or to CD16a.

In some embodiments of these single-chain chimeric polypeptides, the first target-binding domain binds specifically to tissue factor, and the second target-binding domain binds specifically to CD16a. In some embodiments of these single-chain chimeric polypeptides, the first target-binding domain binds specifically to CD16a, and the second target-binding domain bind specifically to tissue factor.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein.

In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, one or both of the first target-binding domain and the second target-binding domain is an anti-tissue factor antigen-binding domain or an anti-CD16a antigen-binding domain. In some embodiments of these single-chain chimeric polypeptides, the first target-binding domain and the second target-binding domain are each independently an anti-tissue factor antigen-binding domain or an anti-CD16a antigen-binding domain. In some embodiments of these single-chain chimeric polypeptides, the first target-binding domain is an anti-tissue factor antigen-binding domain and the second target-binding domain is an anti-CD16a antigen-binding domain. In some embodiments of these single-chain chimeric polypeptides, the first target-binding domain is an anti-CD16a antigen-binding domain and the second target-binding domain is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 98, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 98.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 99.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 100.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 101, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 102.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 103.

In some embodiments, the anti-CD16a antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 125.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

### Humanized Anti-Human TFscFv/hTRβC1/Anti-CD16a-scFv

In some embodiments, provided herein is a single-chain chimeric polypeptide that includes: (i) a first target-binding domain that includes a humanized anti-TF scFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-CD16a-scFv (e.g., a sequence that is at least 80%, at least 90%, at least 95%, or 100% identical to SEQ ID NO: 125).

In some embodiments, provided herein is a single-chain chimeric polypeptide that includes: (i) a first target-binding domain that includes a humanized anti-TF scFv (e.g., a sequence that is at least 80%, at least 90%, at least 95%, or 100% identical to SEQ ID NO: 103), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes an anti-CD16a-scFv (e.g., a sequence that is at least 80%, at least 90%, at least 95%, or 100% identical to SEQ ID NO: 125), wherein the first target-binding domain, the soluble T cell receptor β-chain constant region (TRβC) domain, and the second target-binding domain are positioned in a N- to C-terminal direction.

### Exemplary Embodiments of Single-Chain Chimeric Polypeptides—Type I

In some embodiments of any of the single-chain chimeric polypeptides described herein, the first target-binding domain and/or the second target-binding domain can independently bind specifically to a ligand of TGF-βRII or to HIV. In some embodiments of these single-chain chimeric polypeptides, the first target-binding domain and the second target-binding domain both bind specifically to a ligand of TGF-βRII or to HIV.

In some embodiments of these single-chain chimeric polypeptides, the first target-binding domain binds specifically to a ligand of TGF-βRII, and the second target-binding domain binds specifically to HIV. In some embodiments of these single-chain chimeric polypeptides, the first target-binding domain binds specifically to HIV, and the second target-binding domain binds specifically to a ligand of TGF-βRII.

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments, the soluble T cell receptor β-chain constant region (TRβC) domain can be any of the exemplary soluble T cell receptor β-chain constant region (TRβC) domains described herein.

In some embodiments, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, one or both of the first target-binding domain and the second target-binding domain is a soluble TGFβRII receptor or an anti-HIV antigen-binding domain. In some embodiments of these single-chain chimeric polypeptides, the first target-binding domain and the second target-binding domain are each independently a soluble TGFβRII receptor or an anti-HIV antigen-binding domain. In some embodiments of these single-chain chimeric polypeptides, the first target-binding domain is a soluble TGFβRII receptor and the second target-binding domain is an anti-HIV antigen-binding domain. In some embodiments of these single-chain chimeric polypeptides, the first target-binding domain is an anti-HIV antigen-binding domain and the second target-binding domain is a soluble TGFβRII receptor.

In some embodiments, the soluble TGFβRII includes a first sequence of soluble human TGFβRII and a second sequence of soluble human TGFβRII. In some embodiments, the soluble TGFβRII includes a linker disposed between the first sequence of soluble human TGFβRII and the second sequence of soluble human TGFβRII. In some examples of these single-chain chimeric polypeptides, the linker includes the sequence GGGGSGGGGSGGGGS (SEQ ID NO: 53)

A non-limiting example of a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10, SEQ ID NO: 40, or SEQ ID NO: 92.

In some embodiments, the anti-HIV antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 127.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-CD3 antigen-binding domain.

In some embodiments of these single-chain chimeric polypeptides, the anti-CD3 antigen-binding domain includes or is an anti-CD3 scFv. A non-limiting example of an anti-CD3 scFv includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 93.

In some embodiments, an anti-CD3 scFv can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 94.

In some embodiments, an anti-CD3 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 95.

In some embodiments, an anti-CD3 scFv is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 96.

In some embodiments, an anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 41 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 42.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 45.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 97.

In some embodiments, the anti-CD3 antigen-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 137.

### TGFβRII/TRβC/Humanized Anti-Human HIVscFv

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a humanized anti-human HIVscFv (e.g., a sequence that is at least 80%, at least 90%, at least 95%, or 100% identical to SEQ ID NO: 127).

In some embodiments, the single-chain chimeric polypeptide further includes an additional target-binding domain.

In some embodiments, the additional target-binding domain includes or is an anti-CD3 antigen-binding domain. In some embodiments, the anti-CD3 antigen-binding domain includes an anti-CD3scFv (e.g., a sequence that is at least 80%, at least 90%, at least 95%, or 100% identical to any one of SEQ ID NO: 93, SEQ ID NO: 95, and SEQ ID NO: 97).

In some embodiments, the single-chain chimeric polypeptide further includes a signal sequence at its N-terminal end. In some embodiments the signal peptide sequence is MKWVTFISLLFLESSAYS (SEQ ID NO: 57). In some embodiments the signal peptide sequence is MGVKVLFALICIAVAEA (SEQ ID NO: 129).

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a humanized anti-human HIVscFv (e.g., a sequence that is at least 80%, at least 90%, at least 95%, or 100% identical to SEQ ID NO: 127), and (iv) an additional target-binding domain that includes an anti-CD3 scFv (e.g., a sequence that is at least 80%, at least 90%, at least 95%, or 100% identical to SEQ ID NO: 93, SEQ ID NO: 95, and SEQ ID NO: 97), wherein the first target-binding domain, the soluble T cell receptor β-chain constant region (TRβC) domain, the second target-binding domain, and the additional target-binding domain are positioned in a N- to C-terminal direction.

### Exemplary Embodiments of Single-Chain Chimeric Polypeptides—Type J

In some embodiments of any of the single-chain chimeric polypeptides described herein, the first target-binding domain and/or the second target-binding domain can independently bind specifically to an IL-15 receptor (e.g., human IL-15 receptor) or a ligand of TGF-βRII (e.g., a human a ligand of TGFβRII receptor). In some embodiments, the first target-binding domain binds specifically to an IL-15 receptor (e.g., human IL-15 receptor) and the second target-binding domain binds specifically to a ligand of TGF-βRII (e.g., a human a ligand of TGFβRII receptor). In some embodiments, the first target-binding domain binds specifically to a ligand of TGF-βRII (e.g., a human a ligand of TGFβRII receptor) and the second target-binding domain binds specifically to an IL-15 receptor (e.g., human IL-15 receptor).

In some embodiments of these single-chain chimeric polypeptides, the first target-binding domain and the soluble TRβC domain directly abut each other. In some embodiments of these single-chain chimeric polypeptides, the single-chain chimeric polypeptide further includes a linker sequence (e.g., any of the exemplary linkers described herein) between the first target-binding domain and the soluble TRβC domain.

In some embodiments of these single-chain chimeric polypeptides, the soluble TRβC domain and the second target-binding domain directly abut each other. In some embodiments of these single-chain chimeric polypeptides, the single-chain chimeric polypeptide further includes a linker sequence (e.g., any of the exemplary linkers described herein) between the soluble TRβC domain and the second target-binding domain.

In some embodiments of these single-chain chimeric polypeptides, the soluble T cell receptor β-chain constant region (TRβC) domain can be any of the exemplary soluble T cell receptor β-chain constant region (TRβC) domains described herein. In some embodiments of these single-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these single-chain chimeric polypeptides, the first target-binding domain is a soluble human IL-15 protein and the second target-binding domain is a soluble TGFβRII receptor. In some embodiments of these single-chain chimeric polypeptides, the first target-binding domain is a soluble TGFβRII receptor and the second target-binding domain is a soluble IL-15.

A non-limiting example of an IL-15 that binds specifically to an IL-15 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 22.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these single-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7 and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7. In some embodiments, the soluble TGFβRII receptor can further include a linker sequence (e.g., any of the exemplary linker sequences described herein or known in the art) between the first sequence and the second sequence.

In some embodiments of these single-chain chimeric polypeptides, the soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 37, or SEQ ID NO: 38.

In some embodiments of these single-chain chimeric polypeptides, the soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 39.

In some embodiments of these single-chain chimeric polypeptides, a soluble TGFβRII receptor can be encoded by a nucleic acid including a sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 40.

In some embodiments, the single-chain chimeric polypeptide further comprises one or more additional target-binding domains at the N-terminal end or the C-terminal end of the single-chain chimeric polypeptide.

In some embodiments, the additional target-binding domain and the first target binding domain directly abut each other. In some embodiments, the single-chain chimeric polypeptide further includes a linker sequence (e.g., any of the exemplary linkers described herein) between the additional target-binding domain and the first target binding domain.

In some embodiments, the additional target-binding domain and the second target binding domain directly abut each other. In some embodiments, the single-chain chimeric polypeptide further includes a linker sequence (e.g., any of the exemplary linkers described herein) between the additional target-binding domain and the second target binding domain.

In some embodiments, the additional target-binding domain includes or is an anti-tissue factor antigen-binding domain, an anti-PD-1 antigen-binding domain, an anti-CD3 antigen-binding domain, an anti-CD16a antigen-binding domain, a soluble IL-7, or a soluble IL-21.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), and (iii) a second target-binding domain that includes a soluble human IL-15 protein (e.g., a sequence that is at least 80%, at least 90%, at least 95%, or 100% identical to SEQ ID NO: 22), wherein the first target-binding domain, the soluble T cell receptor β-chain constant region (TRβC) domain, and the second target-binding domain are positioned in a N- to C-terminal direction.

In some embodiments, provided herein is a single-chain chimeric polypeptide that includes: (i) a first target-binding domain that includes a soluble TGFβRII receptor (e.g., a sequence that is at least 80%, at least 90%, at least 95%, or 100% identical to SEQ ID NO: 9 or SEQ ID NO: 39), (ii) a soluble T cell receptor β-chain constant region (TRβC) domain (e.g., a sequence that is at least 80%, at least 90%, at least 95%, or 100% identical to SEQ ID NO: 1 or SEQ ID NO: 3), (iii) a second target-binding domain that includes a soluble human IL-15 protein (e.g., a sequence that is at least 80%, at least 90%, at least 95%, or 100% identical to SEQ ID NO: 22), and (iv) an additional target-binding domain (e.g., any of the additional target-binding domains described herein), wherein the first target-binding domain, the soluble T cell receptor β-chain constant region (TRβC) domain, the second target-binding domain, and the additional target-binding domain are positioned in a N- to C-terminal direction.

### Compositions/Kits

Also provided herein are compositions (e.g., pharmaceutical compositions) that include at least one of any single-chain chimeric polypeptides, any of the cells, or any of the nucleic acids described herein. In some embodiments, the compositions include at least one of any of the single-chain chimeric polypeptides described herein. In some embodiments, the compositions include any of the immune cells (e.g., any of the immune cells described herein, e.g., any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Single or multiple administrations of pharmaceutical compositions can be given to a subject in need thereof depending on for example: the dosage and frequency as required and tolerated by the subject. The formulation should provide a sufficient quantity of active agent to effectively treat, prevent or ameliorate conditions, diseases or symptoms.

Also provided herein are kits that include any of the single-chain chimeric polypeptides, compositions, nucleic acids, or cells (e.g., immune cells) described herein. In some embodiments, the kits can include instructions for performing any of the methods described herein. In some embodiments, the kits can include at least one dose of any of the pharmaceutical compositions described herein.

### Nucleic Acids/Vectors

Also provided herein are nucleic acids that encode any of the single-chain chimeric polypeptides described herein. Also provided herein are vectors that include any of the nucleic acids encoding any of the single-chain chimeric polypeptides described herein.

Any of the vectors described herein can be an expression vector. For example, an expression vector can include a promoter sequence operably linked to the sequence encoding the single-chain chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the single-chain chimeric polypeptides described herein.

### Cells

Also provided herein are cells (e.g., any of the exemplary cells described herein or known in the art) comprising any of the nucleic acids described herein that encode any of the single-chain chimeric polypeptides described herein.

Also provided herein are cells (e.g., any of the exemplary cells described herein or known in the art) that include any of the vectors described herein that encode any of the single-chain chimeric polypeptides described herein.

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., an eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Single-Chain Chimeric Polypeptides

Also provided herein are methods of producing any of the single-chain chimeric polypeptides described herein that include culturing any of the cells described herein in a culture medium under conditions sufficient to result in the production of the single-chain chimeric polypeptide; and recovering the single-chain chimeric polypeptide from the cell and/or the culture medium.

The recovery of the single-chain chimeric polypeptide from a culture medium or a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are single-chain chimeric polypeptides (e.g., any of the single-chain chimeric polypeptides described herein) produced by any of the methods described herein.

### Methods of Stimulating an Immune Cell

Also provided herein are methods of stimulating an immune cell (e.g., any of the exemplary immune cells described herein or known in the art) that include contacting an immune cell with an effective amount of any of the single-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the single-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a memory T cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, or a natural killer cell, or a combination thereof.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

Activation of an immune cell can be determined using methods known in the art. For example, activation of an immune cell can be determined by detecting the levels of cytokines and chemokines that are secreted upon activation of an immune cell. Non-limiting examples of cytokines, chemokines, and regulatory molecules that are secreted or upregulated upon activation of an immune cell include: IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, RORγT, FOXP3, STAT6, and GATA3. The detection of these cytokines and chemokines or regulatory molecules can be performed using an immunoassay (e.g., an enzyme-linked immunosorbent assay) or quantitative PCR. For example, activation of an immune cell can result in an increase of about 1% to about 800% (e.g., about 1% to about 750%, about 1% to about 700%, about 1% to about 650%, about 1% to about 600%, about 1% to about 550%, about 1% to about 500%, about 1% to about 450%, about 1% to about 400%, about 1% to about 350%, about 1% to about 300%, about 1% to about 280%, about 1% to about 260%, about 1% to about 240%, about 1% to about 220%, about 1% to about 200%, about 1% to about 180%, about 1% to about 160%, about 1% to about 140%, about 1% to about 120%, about 1% to about 100%, about 1% to about 90%, about 1% to about 80%, about 1% to about 70%, about 1% to about 60%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 800%, about 5% to about 750%, about 5% to about 700%, about 5% to about 650%, about 5% to about 600%, about 5% to about 550%, about 5% to about 500%, about 5% to about 450%, about 5% to about 400%, about 5% to about 350%, about 5% to about 300%, about 5% to about 280%, about 5% to about 260%, about 5% to about 240%, about 5% to about 220%, about 5% to about 200%, about 5% to about 180%, about 5% to about 160%, about 5% to about 140%, about 5% to about 120%, about 5% to about 100%, about 5% to about 90%, about 5% to about 80%, about 5% to about 70%, about 5% to about 60%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 800%, about 10% to about 750%, about 10% to about 700%, about 10% to about 650%, about 10% to about 600%, about 10% to about 550%, about 10% to about 500%, about 10% to about 450%, about 10% to about 400%, about 10% to about 350%, about 10% to about 300%, about 10% to about 280%, about 10% to about 260%, about 10% to about 240%, about 10% to about 220%, about 10% to about 200%, about 10% to about 180%, about 10% to about 160%, about 10% to about 140%, about 10% to about 120%, about 10% to about 100%, about 10% to about 90%, about 10% to about 80%, about 10% to about 70%, about 10% to about 60%, about 10% to about 50%, about 10% to about 45%, about 10% to about 40%, about 10% to about 35%, about 10% to about 30%, about 10% to about 25%, about 10% to about 20%, about 10% to about 15%, about 15% to about 800%, about 15% to about 750%, about 15% to about 700%, about 15% to about 650%, about 15% to about 600%, about 15% to about 550%, about 15% to about 500%, about 15% to about 450%, about 15% to about 400%, about 15% to about 350%, about 15% to about 300%, about 15% to about 280%, about 15% to about 260%, about 15% to about 240%, about 15% to about 220%, about 15% to about 200%, about 15% to about 180%, about 15% to about 160%, about 15% to about 140%, about 15% to about 120%, about 15% to about 100%, about 15% to about 90%, about 15% to about 80%, about 15% to about 70%, about 15% to about 60%, about 15% to about 50%, about 15% to about 45%, about 15% to about 40%, about 15% to about 35%, about 15% to about 30%, about 15% to about 25%, about 15% to about 20%, about 20% to about 800%, about 20% to about 750%, about 20% to about 700%, about 20% to about 650%, about 20% to about 600%, about 20% to about 550%, about 20% to about 500%, about 20% to about 450%, about 20% to about 400%, about 20% to about 350%, about 20% to about 300%, about 20% to about 280%, about 20% to about 260%, about 20% to about 240%, about 20% to about 220%, about 20% to about 200%, about 20% to about 180%, about 20% to about 160%, about 20% to about 140%, about 20% to about 120%, about 20% to about 100%, about 20% to about 90%, about 20% to about 80%, about 20% to about 70%, about 20% to about 60%, about 20% to about 50%, about 20% to about 45%, about 20% to about 40%, about 20% to about 35%, about 20% to about 30%, about 20% to about 25%, about 25% to about 800%, about 25% to about 750%, about 25% to about 700%, about 25% to about 650%, about 25% to about 600%, about 25% to about 550%, about 25% to about 500%, about 25% to about 450%, about 25% to about 400%, about 25% to about 350%, about 25% to about 300%, about 25% to about 280%, about 25% to about 260%, about 25% to about 240%, about 25% to about 220%, about 25% to about 200%, about 25% to about 180%, about 25% to about 160%, about 25% to about 140%, about 25% to about 120%, about 25% to about 100%, about 25% to about 90%, about 25% to about 80%, about 25% to about 70%, about 25% to about 60%, about 25% to about 50%, about 25% to about 45%, about 25% to about 40%, about 25% to about 35%, about 35% to about 800%, about 35% to about 750%, about 35% to about 700%, about 35% to about 650%, about 35% to about 600%, about 35% to about 550%, about 35% to about 500%, about 35% to about 450%, about 35% to about 400%, about 35% to about 350%, about 35% to about 300%, about 35% to about 280%, about 35% to about 260%, about 35% to about 240%, about 35% to about 220%, about 35% to about 200%, about 35% to about 180%, about 35% to about 160%, about 35% to about 140%, about 35% to about 120%, about 35% to about 100%, about 35% to about 90%, about 35% to about 80%, about 35% to about 70%, about 35% to about 60%, about 35% to about 50%, about 35% to about 45%, about 35% to about 40%, about 40% to about 800%, about 40% to about 750%, about 40% to about 700%, about 40% to about 650%, about 40% to about 600%, about 40% to about 550%, about 40% to about 500%, about 40% to about 450%, about 40% to about 400%, about 40% to about 350%, about 40% to about 300%, about 40% to about 280%, about 40% to about 260%, about 40% to about 240%, about 40% to about 220%, about 40% to about 200%, about 40% to about 180%, about 40% to about 160%, about 40% to about 140%, about 40% to about 120%, about 40% to about 100%, about 40% to about 90%, about 40% to about 80%, about 40% to about 70%, about 40% to about 60%, about 40% to about 50%, about 40% to about 45%, about 45% to about 800%, about 45% to about 750%, about 45% to about 700%, about 45% to about 650%, about 45% to about 600%, about 45% to about 550%, about 45% to about 500%, about 45% to about 450%, about 45% to about 400%, about 45% to about 350%, about 45% to about 300%, about 45% to about 280%, about 45% to about 260%, about 45% to about 240%, about 45% to about 220%, about 45% to about 200%, about 45% to about 180%, about 45% to about 160%, about 45% to about 140%, about 45% to about 120%, about 45% to about 100%, about 45% to about 90%, about 45% to about 80%, about 45% to about 70%, about 45% to about 60%, about 45% to about 50%, about 50% to about 800%, about 50% to about 750%, about 50% to about 700%, about 50% to about 650%, about 50% to about 600%, about 50% to about 550%, about 50% to about 500%, about 50% to about 450%, about 50% to about 400%, about 50% to about 350%, about 50% to about 300%, about 50% to about 280%, about 50% to about 260%, about 50% to about 240%, about 50% to about 220%, about 50% to about 200%, about 50% to about 180%, about 50% to about 160%, about 50% to about 140%, about 50% to about 120%, about 50% to about 100%, about 50% to about 90%, about 50% to about 80%, about 50% to about 70%, about 50% to about 60%, about 60% to about 800%, about 60% to about 750%, about 60% to about 700%, about 60% to about 650%, about 60% to about 600%, about 60% to about 550%, about 60% to about 500%, about 60% to about 450%, about 60% to about 400%, about 60% to about 350%, about 60% to about 300%, about 60% to about 280%, about 60% to about 260%, about 60% to about 240%, about 60% to about 220%, about 60% to about 200%, about 60% to about 180%, about 60% to about 160%, about 60% to about 140%, about 60% to about 120%, about 60% to about 100%, about 60% to about 90%, about 60% to about 80%, about 60% to about 70%, about 70% to about 800%, about 70% to about 750%, about 70% to about 700%, about 70% to about 650%, about 70% to about 600%, about 70% to about 550%, about 70% to about 500%, about 70% to about 450%, about 70% to about 400%, about 70% to about 350%, about 70% to about 300%, about 70% to about 280%, about 70% to about 260%, about 70% to about 240%, about 70% to about 220%, about 70% to about 200%, about 70% to about 180%, about 70% to about 160%, about 70% to about 140%, about 70% to about 120%, about 70% to about 100%, about 70% to about 90%, about 70% to about 80%, about 80% to about 800%, about 80% to about 750%, about 80% to about 700%, about 80% to about 650%, about 80% to about 600%, about 80% to about 550%, about 80% to about 500%, about 80% to about 450%, about 80% to about 400%, about 80% to about 350%, about 80% to about 300%, about 80% to about 280%, about 80% to about 260%, about 80% to about 240%, about 80% to about 220%, about 80% to about 200%, about 80% to about 180%, about 80% to about 160%, about 80% to about 140%, about 80% to about 120%, about 80% to about 100%, about 80% to about 90%, about 90% to about 800%, about 90% to about 750%, about 90% to about 700%, about 90% to about 650%, about 90% to about 600%, about 90% to about 550%, about 90% to about 500%, about 90% to about 450%, about 90% to about 400%, about 90% to about 350%, about 90% to about 300%, about 90% to about 280%, about 90% to about 260%, about 90% to about 240%, about 90% to about 220%, about 90% to about 200%, about 90% to about 180%, about 90% to about 160%, about 90% to about 140%, about 90% to about 120%, about 90% to about 100%, about 100% to about 800%, about 100% to about 750%, about 100% to about 700%, about 100% to about 650%, about 100% to about 600%, about 100% to about 550%, about 100% to about 500%, about 100% to about 450%, about 100% to about 400%, about 100% to about 350%, about 100% to about 300%, about 100% to about 280%, about 100% to about 260%, about 100% to about 240%, about 100% to about 220%, about 100% to about 200%, about 100% to about 180%, about 100% to about 160%, about 100% to about 140%, about 100% to about 120%, about 120% to about 800%, about 120% to about 750%, about 120% to about 700%, about 120% to about 650%, about 120% to about 600%, about 120% to about 550%, about 120% to about 500%, about 120% to about 450%, about 120% to about 400%, about 120% to about 350%, about 120% to about 300%, about 120% to about 280%, about 120% to about 260%, about 120% to about 240%, about 120% to about 220%, about 120% to about 200%, about 120% to about 180%, about 120% to about 160%, about 120% to about 140%, about 140% to about 800%, about 140% to about 750%, about 140% to about 700%, about 140% to about 650%, about 140% to about 600%, about 140% to about 550%, about 140% to about 500%, about 140% to about 450%, about 140% to about 400%, about 140% to about 350%, about 140% to about 300%, about 140% to about 280%, about 140% to about 260%, about 140% to about 240%, about 140% to about 220%, about 140% to about 200%, about 140% to about 180%, about 140% to about 160%, about 160% to about 800%, about 160% to about 750%, about 160% to about 700%, about 160% to about 650%, about 160% to about 600%, about 160% to about 550%, about 160% to about 500%, about 160% to about 450%, about 160% to about 400%, about 160% to about 350%, about 160% to about 300%, about 160% to about 280%, about 160% to about 260%, about 160% to about 240%, about 160% to about 220%, about 160% to about 200%, about 160% to about 180%, about 180% to about 800%, about 180% to about 750%, about 180% to about 700%, about 180% to about 650%, about 180% to about 600%, about 180% to about 550%, about 180% to about 500%, about 180% to about 450%, about 180% to about 400%, about 180% to about 350%, about 180% to about 300%, about 180% to about 280%, about 180% to about 260%, about 180% to about 240%, about 180% to about 220%, about 180% to about 200%, about 200% to about 800%, about 200% to about 750%, about 200% to about 700%, about 200% to about 650%, about 200% to about 600%, about 200% to about 550%, about 200% to about 500%, about 200% to about 450%, about 200% to about 400%, about 200% to about 350%, about 200% to about 300%, about 200% to about 280%, about 200% to about 260%, about 200% to about 240%, about 200% to about 220%, about 220% to about 800%, about 220% to about 750%, about 220% to about 700%, about 220% to about 650%, about 220% to about 600%, about 220% to about 550%, about 220% to about 500%, about 220% to about 450%, about 220% to about 400%, about 220% to about 350%, about 220% to about 300%, about 220% to about 280%, about 220% to about 260%, about 220% to about 240%, about 240% to about 800%, about 240% to about 750%, about 240% to about 700%, about 240% to about 650%, about 240% to about 600%, about 240% to about 550%, about 240% to about 500%, about 240% to about 450%, about 240% to about 400%, about 240% to about 350%, about 240% to about 300%, about 240% to about 280%, about 240% to about 260%, about 260% to about 800%, about 260% to about 750%, about 260% to about 700%, about 260% to about 650%, about 260% to about 600%, about 260% to about 550%, about 260% to about 500%, about 260% to about 450%, about 260% to about 400%, about 260% to about 350%, about 260% to about 300%, about 260% to about 280%, about 280% to about 800%, about 280% to about 750%, about 280% to about 700%, about 280% to about 650%, about 280% to about 600%, about 280% to about 550%, about 280% to about 500%, about 280% to about 450%, about 280% to about 400%, about 280% to about 350%, about 280% to about 300%, about 300% to about 800%, about 300% to about 750%, about 300% to about 700%, about 300% to about 650%, about 300% to about 600%, about 300% to about 550%, about 300% to about 500%, about 300% to about 450%, about 300% to about 400%, about 300% to about 350%, about 350% to about 800%, about 350% to about 750%, about 350% to about 700%, about 350% to about 650%, about 350% to about 600%, about 350% to about 550%, about 350% to about 500%, about 350% to about 450%, about 350% to about 400%, about 400% to about 800%, about 400% to about 750%, about 400% to about 700%, about 400% to about 650%, about 400% to about 600%, about 400% to about 550%, about 400% to about 500%, about 400% to about 450%, about 450% to about 800%, about 450% to about 750%, about 450% to about 700%, about 450% to about 650%, about 450% to about 600%, about 450% to about 550%, about 450% to about 500%, about 500% to about 800%, about 500% to about 750%, about 500% to about 700%, about 500% to about 650%, about 500% to about 600%, about 500% to about 550%, about 550% to about 800%, about 550% to about 750%, about 550% to about 700%, about 550% to about 650%, about 550% to about 600%, about 600% to about 800%, about 600% to about 750%, about 600% to about 700%, about 600% to about 650%, about 650% to about 800%, about 650% to about 750%, about 650% to about 700%, about 700% to about 800%, about 700% to about 750%, or about 750% to about 800%) of one or more of any of the cytokines or chemokines or regulatory molecules described herein (e.g., one or more of any of IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, RORγT, FOXP3, STAT6, and GATA3) (e.g., as compared to the level of the one or more cytokines and chemokines in a control not contacted with any of the single-chain chimeric polypeptides described herein).

### Methods of Inducing or Increasing Proliferation of an Immune Cell

Also provided herein are methods of inducing or increasing proliferation of an immune cell (e.g., any of the exemplary immune cells described herein or known in the art) that include contacting an immune cell with an effective amount of any of the single-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the single-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a memory T cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, or a natural killer cell, or a combination thereof.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

Detection of the proliferation of an immune cell can be performed using methods known in the art, e.g., cytometry (e.g., fluorescence-assisted flow cytometry), microscopy, and immunofluorescence microscopy, e.g., by comparing the rate of increase in the concentration of the immune cell in a sample not contacted with a single-chain chimeric polypeptide to the rate of increase in the concentration of the immune cell in a similar sample contacted with any of the single-chain chimeric polypeptides described herein).

In other examples, the proliferation of an immune cell can be indirectly detected by detecting an increase in the level of one or more cytokines or chemokines secreted or regulatory molecules by proliferating immune cells (e.g., one or more of IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, RORγT, FOXP3, STAT6, and GATA3) (e.g., as compared to the level of the one or more cytokines and chemokines in a control not contacted with any of the single-chain chimeric polypeptides described herein).

In some embodiments, the methods provided herein can result in an increase (e.g., about 1% to about 800% increase, or any of the subranges of this range described herein) in the rate of increase in the concentration of the immune cell in a sample contacted with any of the single-chain chimeric polypeptides described herein as compared to the rate of increase in a similar control sample not contacted with any of the single-chain chimeric polypeptides described herein.

### Methods of Inducing Differentiation of an Immune Cell

Also provided herein are method of inducing differentiation of an immune cell (e.g., any of the exemplary immune cells described herein or known in the art) into a memory or memory-like immune cell that include contacting an immune cell with an effective amount of any of the single-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the single-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a senescent CD8⁺ T cell, a mast cell, a macrophage, a neutrophil, a dendritic cell, a basophil, an eosinophil, or a natural killer cell, or a combination thereof.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

In some examples, the immune cell is a NK cell, and the detection of a memory NK cell can include, e.g., the detection of the increased level of one or more of CD25, CD69, CD62L, IL-12, IL-18, IL-33, STAT4, STAT5, Zbtb32, DNAM-1, NKp30, NKp40, NKp46, BIM, Noxa, SOCS1, BNIP3, BNIP3L, IFN-γ, CXCL16, CXCR6, NKG2D, TRAIL, CD49, Ly49D, CD49b, and Ly79H. A description of NK memory cells and methods of detecting the same is described in O'Sullivan et al., Immunity 43:634-645, 2015.

In some examples, the immune cell is a T cell, and the detection of memory T cells can include, e.g., the detection of the level of expression of one or more of CD45RO, CCR7, L-selectin (CD62L), CD44, CD45RA, integrin αeβ7, CD43, CD27, CD28, CD127 (IL-7Rα), CD69, CD95, IL-2Rβ, KLRG-1, LY108, CCR7, CXCR3, CXCR5, CX3CR1, PD-1, TIM3, TIGIT, LAG-3, CTLA-4 and LFA-1. In some examples, the immune cell is a B cell and the detection of memory B cells can include, e.g., the detection of the level of expression of CD27. Other types and markers of memory or memory-like immune cells are known in the art.

### Methods of Killing a Cancer Cell, an Infected Cell, or a Senescent Cell

Also provided herein are methods of killing a cancer cell (e.g., any of the exemplary types of cancer described herein or known in the art), an infected cell (e.g., a cell infected with any of the exemplary viruses described herein or known in the art), or a senescent cell (e.g., a senescent cancer cell, a senescent fibroblast, or a senescent endothelial cell) in a subject in need thereof (e.g., any of the exemplary subjects described herein or known in the art) that include administering to the subject a therapeutically effective amount of any of the single-chain chimeric polypeptides described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples of these methods, the subject has been identified or diagnosed as having an aging-related disease or condition. Non-limiting examples of aging-related diseases and conditions include Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples of these methods, the subject has been diagnosed or identified as having an infectious disease. Non-limiting examples of an infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus.

### Senescent Cells

Senescence is a form of irreversible growth arrest accompanied by phenotypic changes, resistance to apoptosis and activation of damage-sensing signaling pathways. Cellular senescence was first described in cultured human fibroblast cells that lost their ability to proliferate, reaching permanent arrest after about 50 population doublings (referred to as the Hayflick limit). Senescence is considered a stress response that can be induced by a wide range of intrinsic and extrinsic insults, including oxidative and genotoxic stress, DNA damage, telomere attrition, oncogenic activation, mitochondrial dysfunction, or chemotherapeutic agents.

Senescent cells remain metabolically active and can influence the tissue hemostasis, disease and aging through their secretory phenotype. Senescence is considered as a physiologic process and is important in promoting wound healing, tissue homeostasis, regeneration, and fibrosis regulation. For instance, transient induction of senescent cells is observed during would healing and contributes to wound resolution. Perhaps one of the most important roles of senescence is its role in tumor suppression. However, the accumulation of senescent cells also drives aging- and aging-related diseases and conditions. The senescent phenotype also can trigger chronic inflammatory responses and consequently augment chronic inflammatory conditions to promote tumor growth. The connection between senescence and aging was initially based on observations that senescent cells accumulate in aged tissue. The use of transgenic models has enabled the detection of senescent cells systematically in many age-related pathologies. Strategies to selectively eliminate senescent cells has demonstrated that senescent cells can indeed play a causal role in aging and related pathologies.

Senescent cells display important and unique properties which include changes in morphology, chromatin organization, gene expression, and metabolism. There are several biochemical and functional properties associated with cellular senescence, such as (i) increased expression of p16 and p21, inhibitors of cyclin-dependent kinases, (ii) presence of senescence-associated β-galactosidase, a marker of lysosomal activity, (iii) appearance of senescence-associated heterochromatin foci and downregulation of lamin B1 levels, (iv) resistance to apoptosis caused by an increased expression of anti-apoptotic BCL-family protein, and (v) upregulation of CD26 (DPP4), CD36 (Scavenger receptor), forkhead box 4 (FOXO4), PD-L1, and secretory carrier membrane protein 4 (SCAMP4). Senescent cells also express an inflammatory signature, the so-called senescence-associated secretory phenotype (SASP). Through SASP, the senescent cells produce a wide range of inflammatory cytokines (IL-6, IL-8), growth factors (TGF-β), chemokines (CCL-2), and matrix metalloproteinases (MMP-3, MMP-9) that operate in a cell-autonomous manner to reinforce senescence (autocrine effects) and communicate with and modify the microenvironment (paracrine effects). SASP factors can contribute to tumor suppression by triggering senescence surveillance, an immune-mediated clearance of senescent cells. However, chronic inflammation is also a known driver of tumorigenesis, and accumulating evidence indicates that chronic SASP can also boost cancer and aging-related diseases.

The secretion profile of senescent cells is context dependent. For instance, the mitochondrial dysfunction-associated senescence (MiDAS), induced by different mitochondrial dysfunction in human fibroblasts, led to the appearance of a SASP that was deficient in IL-1-dependent inflammatory factors. A decrease in the NAD+/NADH ratio activated AMPK signaling which induced MiDAS through the activation of p53. As a result, p53 inhibited NF-κB signaling which is a crucial inducer of pro-inflammatory SASP. In contrast, the cellular senescence caused by persistent DNA damage in human cells induced an inflammatory SASP, which was dependent on the activation of ataxia-telangiectasia mutated (ATM) kinase but not on that of p53. In particular, the expression and secretion levels of IL-6 and IL-8 were increased. It was also demonstrated that cellular senescence caused by the ectopic expression p16INK4a and p21CIP1 induced the senescent phenotype in human fibroblasts without an inflammatory SASP indicating that the growth arrest itself did not stimulate SASP.

One of the most defining characteristics of senescence is stable growth arrest. This is achieved by two important pathways, the p16/Rb and the p53/p21, both of which are central in tumor suppression. DNA damage results in: (1) high deposition of γH2Ax (histone coding gene) and 53BP1 (involved in DNA damage response) in chromatin: this leads to activation of a kinase cascade eventually resulting in p53 activation, and (2) activation of p16INK4a and ARF (both encoded by CDKN2A) and P15INK4b (encoded by CDKN2B): p53 induces transcription of cyclin-dependent kinase inhibitor (p21) and along with both p16INK4a and p15INK4b block genes for cell cycle progression (CDK4 and CDK6). This eventually leads to hypophosphorylation of Retinoblastoma protein (Rb) and cell cycle arrest at the G1 phase.

Selectively killing senescent cells has been shown to significantly improve the health span of mice in the context of normal aging and ameliorates the consequences of age-related disease or cancer therapy (Ovadya, J Clin Invest. 128(4): 1247-1254, 2018). In nature, the senescent cells are normally removed by the innate immune cells. Induction of senescence not only prevents the potential proliferation and transformation of damaged/altered cells, but also favors tissue repair through the production of SASP factors that function as chemoattractants mainly for Natural Killer (NK) cells (such as IL-15 and CCL2) and macrophages (such as CFS-1 and CCL2). These innate immune cells mediate the immunosurveillance mechanism for eliminating stressed cells. Senescent cells usually up-regulate the NK-cell activating receptor NKG2D and DNAM1 ligands, which belong to a family of stress-inducible ligands: an important component of the frontline immune defense against infectious diseases and malignancies. Upon receptor activation, NK cells can then specifically induce the death of senescent cells through their cytolytic machinery. A role for NK cells in the immune surveillance of senescent cells has been pointed out in liver fibrosis (Sagiv, Oncogene 32(15): 1971-1977, 2013), hepatocellular carcinoma (Iannello, J Exp Med 210(10): 2057-2069, 2013), multiple myeloma (Soriani, Blood 113(15): 3503-3511, 2009), and glioma cells stressed by dysfunction of the mevalonate pathway (Ciaglia, Int J Cancer 142(1): 176-190, 2018). Endometrial cells undergo acute cellular senescence and do not differentiate into decidual cells. The differentiated decidual cells secrete IL-15 and thereby recruit uterine NK cells to target and eliminate the undifferentiated senescent cells thus helping to re-model and rejuvenate the endometrium (Brighton, Elife 6: e31274, 2017). With a similar mechanism, during liver fibrosis, p53-expressing senescent liver satellite cells skewed the polarization of resident Kupfer macrophages and freshly infiltrated macrophages toward the pro-inflammatory M1 phenotype, which display senolytic activity. F4/80+ macrophages have been shown to play a key role in the clearance of mouse uterine senescent cells to maintain postpartum uterine function.

Senescent cells recruit NK cells by mainly upregulating ligands to NKG2D (expressed on NK cells), chemokines, and other SASP factors. In vivo models of liver fibrosis have shown effective clearance of senescent cells by activated NK cells (Krizhanovsky, Cell 134(4): 657-667, 2008). Studies have described various models to study senescence including liver fibrosis (Krizhanovsky, Cell 134(4): 657-667, 2008), osteoarthritis (Xu, J Gerontol A Biol Sci Med Sci 72(6): 780-785, 2017), and Parkinson's disease (Chinta, Cell Rep 22(4): 930-940, 2018). Animal models for studying senescent cells are described in: Krizhanovsky, Cell 134(4): 657-667, 2008; Baker, Nature 479(7372): 232-236, 2011; Farr, Nat Med 23(9): 1072-1079, 2017; Bourgeois, FEBS Lett 592(12): 2083-2097, 2018; Xu, Nat Med 24(8): 1246-1256, 2018).

### Methods of Treatment

Also provided herein are methods of treating a subject in need thereof (e.g., any of the exemplary subjects described herein or known in the art) that include administering to the subject a therapeutically effective amount of any of the single-chain chimeric polypeptides described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-cancer, liver cancer, and hepatocellular carcinoma. In some embodiments, these methods can result in a reduction in the number, severity, or frequency of one or more symptoms of the cancer in the subject (e.g., as compared to the number, severity, or frequency of the one or more symptoms of the cancer in the subject prior to treatment). In some embodiments, these methods can result in a reduction (e.g., about 1% reduction to about 99% reduction, about 1% reduction to about 95% reduction, about 1% reduction to about 90% reduction, about 1% reduction to about 85% reduction, about 1% reduction to about 80% reduction, about 1% reduction to about 75% reduction, about 1% reduction to about 70% reduction, about 1% reduction to about 65% reduction, about 1% reduction to about 60% reduction, about 1% reduction to about 55% reduction, about 1% reduction to about 50% reduction, about 1% reduction to about 45% reduction, about 1% reduction to about 40% reduction, about 1% reduction to about 35% reduction, about 1% reduction to about 30% reduction, about 1% reduction to about 25% reduction, about 1% reduction to about 20% reduction, about 1% reduction to about 15% reduction, about 1% reduction to about 10% reduction, about 1% reduction to about 5% reduction, about 5% reduction to about 99% reduction, about 5% reduction to about 95% reduction, about 5% reduction to about 90% reduction, about 5% reduction to about 85% reduction, about 5% reduction to about 80% reduction, about 5% reduction to about 75% reduction, about 5% reduction to about 70% reduction, about 5% reduction to about 65% reduction, about 5% reduction to about 60% reduction, about 5% reduction to about 55% reduction, about 5% reduction to about 50% reduction, about 5% reduction to about 45% reduction, about 5% reduction to about 40% reduction, about 5% reduction to about 35% reduction, about 5% reduction to about 30% reduction, about 5% reduction to about 25% reduction, about 5% reduction to about 20% reduction, about 5% reduction to about 15% reduction, about 5% reduction to about 10% reduction, about 10% reduction to about 99% reduction, about 10% reduction to about 95% reduction, about 10% reduction to about 90% reduction, about 10% reduction to about 85% reduction, about 10% reduction to about 80% reduction, about 10% reduction to about 75% reduction, about 10% reduction to about 70% reduction, about 10% reduction to about 65% reduction, about 10% reduction to about 60% reduction, about 10% reduction to about 55% reduction, about 10% reduction to about 50% reduction, about 10% reduction to about 45% reduction, about 10% reduction to about 40% reduction, about 10% reduction to about 35% reduction, about 10% reduction to about 30% reduction, about 10% reduction to about 25% reduction, about 10% reduction to about 20% reduction, about 10% reduction to about 15% reduction, about 15% reduction to about 99% reduction, about 15% reduction to about 95% reduction, about 15% reduction to about 90% reduction, about 15% reduction to about 85% reduction, about 15% reduction to about 80% reduction, about 15% reduction to about 75% reduction, about 15% reduction to about 70% reduction, about 15% reduction to about 65% reduction, about 15% reduction to about 60% reduction, about 15% reduction to about 55% reduction, about 15% reduction to about 50% reduction, about 15% reduction to about 45% reduction, about 15% reduction to about 40% reduction, about 15% reduction to about 35% reduction, about 15% reduction to about 30% reduction, about 15% reduction to about 25% reduction, about 15% reduction to about 20% reduction, about 20% reduction to about 99% reduction, about 20% reduction to about 95% reduction, about 20% reduction to about 90% reduction, about 20% reduction to about 85% reduction, about 20% reduction to about 80% reduction, about 20% reduction to about 75% reduction, about 20% reduction to about 70% reduction, about 20% reduction to about 65% reduction, about 20% reduction to about 60% reduction, about 20% reduction to about 55% reduction, about 20% reduction to about 50% reduction, about 20% reduction to about 45% reduction, about 20% reduction to about 40% reduction, about 20% reduction to about 35% reduction, about 20% reduction to about 30% reduction, about 20% reduction to about 25% reduction, about 25% reduction to about 99% reduction, about 25% reduction to about 95% reduction, about 25% reduction to about 90% reduction, about 25% reduction to about 85% reduction, about 25% reduction to about 80% reduction, about 25% reduction to about 75% reduction, about 25% reduction to about 70% reduction, about 25% reduction to about 65% reduction, about 25% reduction to about 60% reduction, about 25% reduction to about 55% reduction, about 25% reduction to about 50% reduction, about 25% reduction to about 45% reduction, about 25% reduction to about 40% reduction, about 25% reduction to about 35% reduction, about 25% reduction to about 30% reduction, about 30% reduction to about 99% reduction, about 30% reduction to about 95% reduction, about 30% reduction to about 90% reduction, about 30% reduction to about 85% reduction, about 30% reduction to about 80% reduction, about 30% reduction to about 75% reduction, about 30% reduction to about 70% reduction, about 30% reduction to about 65% reduction, about 30% reduction to about 60% reduction, about 30% reduction to about 55% reduction, about 30% reduction to about 50% reduction, about 30% reduction to about 45% reduction, about 30% reduction to about 40% reduction, about 30% reduction to about 35% reduction, about 35% reduction to about 99% reduction, about 35% reduction to about 95% reduction, about 35% reduction to about 90% reduction, about 35% reduction to about 85% reduction, about 35% reduction to about 80% reduction, about 35% reduction to about 75% reduction, about 35% reduction to about 70% reduction, about 35% reduction to about 65% reduction, about 35% reduction to about 60% reduction, about 35% reduction to about 55% reduction, about 35% reduction to about 50% reduction, about 35% reduction to about 45% reduction, about 35% reduction to about 40% reduction, about 40% reduction to about 99% reduction, about 40% reduction to about 95% reduction, about 40% reduction to about 90% reduction, about 40% reduction to about 85% reduction, about 40% reduction to about 80% reduction, about 40% reduction to about 75% reduction, about 40% reduction to about 70% reduction, about 40% reduction to about 65% reduction, about 40% reduction to about 60% reduction, about 40% reduction to about 55% reduction, about 40% reduction to about 50% reduction, about 40% reduction to about 45% reduction, about 45% reduction to about 99% reduction, about 45% reduction to about 95% reduction, about 45% reduction to about 90% reduction, about 45% reduction to about 85% reduction, about 45% reduction to about 80% reduction, about 45% reduction to about 75% reduction, about 45% reduction to about 70% reduction, about 45% reduction to about 65% reduction, about 45% reduction to about 60% reduction, about 45% reduction to about 55% reduction, about 45% reduction to about 50% reduction, about 50% reduction to about 99% reduction, about 50% reduction to about 95% reduction, about 50% reduction to about 90% reduction, about 50% reduction to about 85% reduction, about 50% reduction to about 80% reduction, about 50% reduction to about 75% reduction, about 50% reduction to about 70% reduction, about 50% reduction to about 65% reduction, about 50% reduction to about 60% reduction, about 50% reduction to about 55% reduction, about 55% reduction to about 99% reduction, about 55% reduction to about 95% reduction, about 55% reduction to about 90% reduction, about 55% reduction to about 85% reduction, about 55% reduction to about 80% reduction, about 55% reduction to about 75% reduction, about 55% reduction to about 70% reduction, about 55% reduction to about 65% reduction, about 55% reduction to about 60% reduction, about 60% reduction to about 99% reduction, about 60% reduction to about 95% reduction, about 60% reduction to about 90% reduction, about 60% reduction to about 85% reduction, about 60% reduction to about 80% reduction, about 60% reduction to about 75% reduction, about 60% reduction to about 70% reduction, about 60% reduction to about 65% reduction, about 65% reduction to about 99% reduction, about 65% reduction to about 95% reduction, about 65% reduction to about 90% reduction, about 65% reduction to about 85% reduction, about 65% reduction to about 80% reduction, about 65% reduction to about 75% reduction, about 65% reduction to about 70% reduction, about 70% reduction to about 99% reduction, about 70% reduction to about 95% reduction, about 70% reduction to about 90% reduction, about 70% reduction to about 85% reduction, about 70% reduction to about 80% reduction, about 70% reduction to about 75% reduction, about 75% reduction to about 99% reduction, about 75% reduction to about 95% reduction, about 75% reduction to about 90% reduction, about 75% reduction to about 85% reduction, about 75% reduction to about 80% reduction, about 80% reduction to about 99% reduction, about 80% reduction to about 95% reduction, about 80% reduction to about 90% reduction, about 80% reduction to about 85% reduction, about 85% reduction to about 99% reduction, about 85% reduction to about 95% reduction, about 85% reduction to about 90% reduction, about 90% reduction to about 99% reduction, about 90% reduction to about 95% reduction, or about 95% reduction to about 99% reduction) in the volume of one or more solid tumors in the subject (e.g., as compared to the volume of the one or more solid tumors prior to treatment or at the start of treatment). In some embodiments, the these methods can reduce (e.g., about 1% reduction to about 99% reduction, or any of the subranges of this range described herein) the risk of developing a metastasis or developing one or more additional metastasis in a subject (e.g., as compared to the risk of developing a metastasis or developing one or more additional metastasis in a subject prior to treatment or in a similar subject or a population of subjects administered a different treatment).

In some examples of these methods, the subject has been identified or diagnosed as having an aging-related disease or condition. Non-limiting examples of aging-related diseases and conditions include Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction. In some examples, these methods can result in a reduction in the number, severity, or frequency of one or more symptoms of the aging-related disease or condition in the subject (e.g., as compared to the number, severity, or frequency of the one or more symptoms of the aging-related disease or condition in the subject prior to treatment). In some examples, the methods can result in a decrease (e.g., about 1% decrease to about 99% decrease, an about 1% decrease to about 95% decrease, about 1% decrease to about 90% decrease, about 1% decrease to about 85% decrease, about 1% decrease to about 80% decrease, about 1% decrease to about 75% decrease, about 1% to about 70% decrease, about 1% decrease to about 65% decrease, about 1% decrease to about 60% decrease, about 1% decrease to about 55% decrease, about 1% decrease to about 50% decrease, about 1% decrease to about 45% decrease, about 1% decrease to about 40% decrease, about 1% decrease to about 35% decrease, about 1% decrease to about 30% decrease, about 1% decrease to about 25% decrease, about 1% decrease to about 20% decrease, about 1% decrease to about 15% decrease, about 1% decrease to about 10% decrease, about 1% decrease to about 5% decrease, about 5% decrease to about 99% decrease, an about 5% decrease to about 95% decrease, about 5% decrease to about 90% decrease, about 5% decrease to about 85% decrease, about 5% decrease to about 80% decrease, about 5% decrease to about 75% decrease, about 5% to about 70% decrease, about 5% decrease to about 65% decrease, about 5% decrease to about 60% decrease, about 5% decrease to about 55% decrease, about 5% decrease to about 50% decrease, about 5% decrease to about 45% decrease, about 5% decrease to about 40% decrease, about 5% decrease to about 35% decrease, about 5% decrease to about 30% decrease, about 5% decrease to about 25% decrease, about 5% decrease to about 20% decrease, about 5% decrease to about 15% decrease, about 5% decrease to about 10% decrease, about 10% decrease to about 99% decrease, an about 10% decrease to about 95% decrease, about 10% decrease to about 90% decrease, about 10% decrease to about 85% decrease, about 10% decrease to about 80% decrease, about 10% decrease to about 75% decrease, about 10% to about 70% decrease, about 10% decrease to about 65% decrease, about 10% decrease to about 60% decrease, about 10% decrease to about 55% decrease, about 10% decrease to about 50% decrease, about 10% decrease to about 45% decrease, about 10% decrease to about 40% decrease, about 10% decrease to about 35% decrease, about 10% decrease to about 30% decrease, about 10% decrease to about 25% decrease, about 10% decrease to about 20% decrease, about 10% decrease to about 15% decrease, about 15% decrease to about 99% decrease, an about 15% decrease to about 95% decrease, about 15% decrease to about 90% decrease, about 15% decrease to about 85% decrease, about 15% decrease to about 80% decrease, about 15% decrease to about 75% decrease, about 15% to about 70% decrease, about 15% decrease to about 65% decrease, about 15% decrease to about 60% decrease, about 15% decrease to about 55% decrease, about 15% decrease to about 50% decrease, about 15% decrease to about 45% decrease, about 15% decrease to about 40% decrease, about 15% decrease to about 35% decrease, about 15% decrease to about 30% decrease, about 15% decrease to about 25% decrease, about 15% decrease to about 20% decrease, about 20% decrease to about 99% decrease, an about 20% decrease to about 95% decrease, about 20% decrease to about 90% decrease, about 20% decrease to about 85% decrease, about 20% decrease to about 80% decrease, about 20% decrease to about 75% decrease, about 20% to about 70% decrease, about 20% decrease to about 65% decrease, about 20% decrease to about 60% decrease, about 20% decrease to about 55% decrease, about 20% decrease to about 50% decrease, about 20% decrease to about 45% decrease, about 20% decrease to about 40% decrease, about 20% decrease to about 35% decrease, about 20% decrease to about 30% decrease, about 20% decrease to about 25% decrease, about 25% decrease to about 99% decrease, an about 25% decrease to about 95% decrease, about 25% decrease to about 90% decrease, about 25% decrease to about 85% decrease, about 25% decrease to about 80% decrease, about 25% decrease to about 75% decrease, about 25% to about 70% decrease, about 25% decrease to about 65% decrease, about 25% decrease to about 60% decrease, about 25% decrease to about 55% decrease, about 25% decrease to about 50% decrease, about 25% decrease to about 45% decrease, about 25% decrease to about 40% decrease, about 25% decrease to about 35% decrease, about 25% decrease to about 30% decrease, about 30% decrease to about 99% decrease, an about 30% decrease to about 95% decrease, about 30% decrease to about 90% decrease, about 30% decrease to about 85% decrease, about 30% decrease to about 80% decrease, about 30% decrease to about 75% decrease, about 30% to about 70% decrease, about 30% decrease to about 65% decrease, about 30% decrease to about 60% decrease, about 30% decrease to about 55% decrease, about 30% decrease to about 50% decrease, about 30% decrease to about 45% decrease, about 30% decrease to about 40% decrease, about 30% decrease to about 35% decrease, about 35% decrease to about 99% decrease, an about 35% decrease to about 95% decrease, about 35% decrease to about 90% decrease, about 35% decrease to about 85% decrease, about 35% decrease to about 80% decrease, about 35% decrease to about 75% decrease, about 35% to about 70% decrease, about 35% decrease to about 65% decrease, about 35% decrease to about 60% decrease, about 35% decrease to about 55% decrease, about 35% decrease to about 50% decrease, about 35% decrease to about 45% decrease, about 35% decrease to about 40% decrease, about 40% decrease to about 99% decrease, an about 40% decrease to about 95% decrease, about 40% decrease to about 90% decrease, about 40% decrease to about 85% decrease, about 40% decrease to about 80% decrease, about 40% decrease to about 75% decrease, about 40% to about 70% decrease, about 40% decrease to about 65% decrease, about 40% decrease to about 60% decrease, about 40% decrease to about 55% decrease, about 40% decrease to about 50% decrease, about 40% decrease to about 45% decrease, about 45% decrease to about 99% decrease, an about 45% decrease to about 95% decrease, about 45% decrease to about 90% decrease, about 45% decrease to about 85% decrease, about 45% decrease to about 80% decrease, about 45% decrease to about 75% decrease, about 45% to about 70% decrease, about 45% decrease to about 65% decrease, about 45% decrease to about 60% decrease, about 45% decrease to about 55% decrease, about 45% decrease to about 50% decrease, about 50% decrease to about 99% decrease, an about 50% decrease to about 95% decrease, about 50% decrease to about 90% decrease, about 50% decrease to about 85% decrease, about 50% decrease to about 80% decrease, about 50% decrease to about 75% decrease, about 50% to about 70% decrease, about 50% decrease to about 65% decrease, about 50% decrease to about 60% decrease, about 50% decrease to about 55% decrease, about 55% decrease to about 99% decrease, an about 55% decrease to about 95% decrease, about 55% decrease to about 90% decrease, about 55% decrease to about 85% decrease, about 55% decrease to about 80% decrease, about 55% decrease to about 75% decrease, about 55% to about 70% decrease, about 55% decrease to about 65% decrease, about 55% decrease to about 60% decrease, about 60% decrease to about 99% decrease, an about 60% decrease to about 95% decrease, about 60% decrease to about 90% decrease, about 60% decrease to about 85% decrease, about 60% decrease to about 80% decrease, about 60% decrease to about 75% decrease, about 60% to about 70% decrease, about 60% decrease to about 65% decrease, about 65% decrease to about 99% decrease, an about 65% decrease to about 95% decrease, about 65% decrease to about 90% decrease, about 65% decrease to about 85% decrease, about 65% decrease to about 80% decrease, about 65% decrease to about 75% decrease, about 65% to about 70% decrease, about 70% decrease to about 99% decrease, an about 70% decrease to about 95% decrease, about 70% decrease to about 90% decrease, about 70% decrease to about 85% decrease, about 70% decrease to about 80% decrease, about 70% decrease to about 75% decrease, about 75% decrease to about 99% decrease, an about 75% decrease to about 95% decrease, about 75% decrease to about 90% decrease, about 75% decrease to about 85% decrease, about 75% decrease to about 80% decrease, about 80% decrease to about 99% decrease, an about 80% decrease to about 95% decrease, about 80% decrease to about 90% decrease, about 80% decrease to about 85% decrease, about 85% decrease to about 99% decrease, an about 85% decrease to about 95% decrease, about 85% decrease to about 90% decrease, about 90% decrease to about 99% decrease, an about 90% decrease to about 95% decrease, or about 95% decrease to about 99% decrease) in the number of senescent cells in the subject (e.g., a decrease in the number of senescent cells in one or more specific tissues involved and/or implicated in the aging-related disease or disorder in the subject), e.g., as compared to the number of senescent cells in the subject prior to treatment.

In some examples of these methods, the subject has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus. In some embodiments, these methods can result in a decrease in the infectious titer (e.g., viral titer) in a subject (e.g., as compared to the infectious titer in the subject prior to treatment). In some embodiments, these methods can result in a reduction in the number, severity, or frequency of one or more symptoms of the infectious disease (e.g., viral infection) in the subject (e.g., as compared to the number, severity, or frequency of the one or more symptoms of the infectious disease in the subject prior to treatment).

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine or primate animals) may be employed.

### Additional Therapeutic Agents

Some embodiments of any of the methods described herein can further include administering to a subject (e.g., any of the subjects described herein) a therapeutically effective amount of one or more additional therapeutic agents. The one or more additional therapeutic agents can be administered to the subject at substantially the same time as a single-chain chimeric polypeptide (e.g., any of the single-chain chimeric polypeptides described herein) or an immune cell (e.g., administered as a single formulation or two or more formulations to the subject). In some embodiments, one or more additional therapeutic agents can be administered to the subject prior to administration of a single-chain chimeric polypeptide (e.g., any of the single-chain chimeric polypeptides described herein) or an immune cell. In some embodiments, one or more additional therapeutic agents can be administered to the subject after administration of a single-chain chimeric polypeptide (e.g., any of the single-chain chimeric polypeptides described herein) or an immune cell to the subject.

Non-limiting examples of additional therapeutic agents include: anti-cancer drugs, activating receptor agonists, immune checkpoint inhibitors, agents for blocking HLA-specific inhibitory receptors, Glucogen Synthase Kinase (GSK) 3 inhibitors, and antibodies.

Non-limiting examples of anticancer drugs include antimetabolic drugs (e.g., 5-fluorouracil (5-FU), 6-mercaptopurine (6-MP), capecitabine, cytarabine, floxuridine, fludarabine, gemcitabine, hydroxycarbamide, methotrexate, 6-thioguanine, cladribine, nelarabine, pentostatin, or pemetrexed), plant alkaloids (e.g., vinblastine, vincristine, vindesine, camptothecin, 9-methoxycamptothecin, coronaridine, taxol, naucleaorals, diprenylated indole alkaloid, montamine, schischkiniin, protoberberine, berberine, sanguinarine, chelerythrine, chelidonine, liriodenine, clivorine, β-carboline, antofine, tylophorine, cryptolepine, neocryptolepine, corynoline, sampangine, carbazole, crinamine, montanine, ellipticine, paclitaxel, docetaxel, etoposide, tenisopide, irinotecan, topotecan, or acridone alkaloids), proteasome inhibitors (e.g., lactacystin, disulfiram, epigallocatechin-3-gallate, marizomib (salinosporamide A), oprozomib (ONX-0912), delanzomib (CEP-18770), epoxomicin, MG132, beta-hydroxy beta-methylbutyrate, bortezomib, carfilzomib, or ixazomib), antitumor antibiotics (e.g., doxorubicin, daunorubicin, epirubicin, mitoxantrone, idarubicin, actinomycin, plicamycin, mitomycin, or bleomycin), histone deacetylase inhibitors (e.g., vorinostat, panobinostat, belinostat, givinostat, abexinostat, depsipeptide, entinostat, phenyl butyrate, valproic acid, trichostatin A, dacinostat, mocetinostat, pracinostat, nicotinamide, cambinol, tenovin 1, tenovin 6, sirtinol, ricolinostat, tefinostat, kevetrin, quisinostat, resminostat, tacedinaline, chidamide, or selisistat), tyrosine kinase inhibitors (e.g., axitinib, dasatinib, encorafinib, erlotinib, imatinib, nilotinib, pazopanib, and sunitinib), and chemotherapeutic agents (e.g., all-trans retinoic acid, azacitidine, azathioprine, doxifluridine, epothilone, hydroxyurea, imatinib, teniposide, tioguanine, valrubicin, vemurafenib, and lenalidomide). Additional examples of chemotherapeutic agents include alkylating agents, e.g., mechlorethamine, cyclophosphamide, chlorambucil, melphalan, ifosfamide, thiotepa, hexamethylmelamine, busulfan, altretamine, procarbazine, dacarbazine, temozolomide, carmustine, lumustine, streptozocin, carboplatin, cisplatin, and oxaliplatin.

Non-limiting examples of activating receptor agonists include any agonists for activating receptors which activate and enhance the cytotoxicity of NK cells, including anti-CD16 antibodies (e.g., anti-CD16/CD30 bispecific monoclonal antibody (BiMAb)) and Fc-based fusion proteins. Non-limiting examples of checkpoint inhibitors include anti-PD-1 antibodies (e.g., MEDI0680), anti-PD-L1 antibodies (e.g., BCD-135, BGB-A333, CBT-502, CK-301, CS1001, FAZ053, KN035, MDX-1105, MSB2311, SHR-1316, anti-PD-L1/CTLA-4 bispecific antibody KN046, anti-PD-L1/TGFβRII fusion protein M7824, anti-PD-L1/TIM-3 bispecific antibody LY3415244, atezolizumab, or avelumab), anti-TIM3 antibodies (e.g., TSR-022, Sym023, or MBG453) and anti-CTLA-4 antibodies (e.g., AGEN1884, MK-1308, or an anti-CTLA-4/OX40 bispecific antibody ATOR-1015). Non-limiting examples of agents for blocking HLA-specific inhibitory receptors include monalizumab (e.g., an anti-HLA-E NKG2A inhibitory receptor monoclonal antibody). Non-limiting examples of GSK3 inhibitor include tideglusib or CHIR99021. Non-limiting examples of antibodies that can be used as additional therapeutic agents include anti-CD26 antibodies (e.g., YS110), anti-CD36 antibodies, and any other antibody or antibody construct that can bind to and activate an Fc receptor (e.g., CD16) on a NK cell. In some embodiments, an additional therapeutic agent can be insulin or metformin.

1. A single-chain chimeric polypeptide comprising:
(i) a first target-binding domain;
(ii) a soluble T cell receptor β-chain constant region (TRβC) domain; and
(iii) a second target-binding domain.

2. The single-chain chimeric polypeptide of claim 1, wherein the first target-binding domain and the soluble TRβC domain directly abut each other.

3. The single-chain chimeric polypeptide of claim 1, wherein the single-chain chimeric polypeptide further comprises a linker sequence between the first target-binding domain and the soluble TRβC domain.

4. The single-chain chimeric polypeptide of claim 1, wherein the soluble TRβC domain and the second target-binding domain directly abut each other.

5. The single-chain chimeric polypeptide of claim 1, wherein the single-chain chimeric polypeptide further comprises a linker sequence between the soluble TRβC domain and the second target-binding domain.

6.-9. (canceled)

10. The single-chain chimeric polypeptide of claim 1, wherein one or both of the first target-binding domain and the second target-binding domain is an antigen-binding domain.

11. The single-chain chimeric polypeptide of claim 10, wherein one or both of the first target-binding domain and the second target-binding domain bind to a target selected from the group consisting of: tissue factor (CD142), CD16a, CD28, CD3, CD33, CD20, CD19, CD22, CD123, IL-1R, IL-1, VEGF, IL-6R, IL-4, IL-10, PDL-1, TIGIT, PD-1, LAC-3, TIM3, CTLA4, MICA, MICB, IL-6, IL-8, TNFα, CD26, CD26a, CD36, ULBP2, CD30, CD40, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein, HLA-DR, DLL4, TYRO3, AXL, MER, CD122, CD155, PDGFDD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for ICOS, a receptor for IL-1, a receptor for IL-2, a receptor for IL-3, a receptor for IL-4, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

12. The single-chain chimeric polypeptide of claim 1, wherein one or both of the first target-binding domain and the second target-binding domain is a soluble interleukin, a soluble cytokine protein, or a soluble cell surface protein.

13. The single-chain chimeric polypeptide of claim 12, wherein the soluble interleukin, soluble cytokine protein, or soluble cell surface protein is selected from the group consisting of: IL-1, IL-2, IL-3, IL-4, IL-7, IL-8, IL-10, IL-12, IL-15, IL-17, IL-18, IL-21, PDGF-D, SCF, FLT3L, MICA, MICB, and a ULP16-binding protein.

14. (canceled)

15. The single-chain chimeric polypeptide of claim 1, wherein one or both of the first target-binding domain and the second target-binding domain is a soluble interleukin receptor, a soluble cytokine receptor, or a soluble cell surface receptor.

16. The single-chain chimeric polypeptide of claim 15, wherein the soluble interleukin receptor, the soluble cytokine receptor, or the soluble cell surface receptor is a soluble TGF-β receptor II (TGF-βRII), a soluble TGF-βRIII, a soluble NKG2D, a soluble NKp30, a soluble NKp44, a soluble NKp46, a soluble DNAM-1, a scMHCI, a scMHCII, a soluble CD155, or a soluble CD28.

17. (canceled)

18. The single-chain chimeric polypeptide of claim 1, wherein:
the first target-binding domain is IL-7 and the second target-binding domain is a soluble TGF-βRII;
the first target-binding domain is IL-15 and the second target-binding domain is a soluble TGF-βRII;
the first target-binding domain is a soluble TGF-βRII and the second target-binding domain is a soluble TGF-βRII; or
the first target-binding domain is IL-7 and the second target-binding domain is a soluble IL-21.

19.-26. (canceled)

27. The single-chain chimeric polypeptide of claim 1, wherein the single-chain chimeric polypeptide further comprises one or more additional target-binding domains at its N- and/or C-terminus.

28. The single-chain chimeric polypeptide of claim 1, wherein the soluble TRβC domain is a soluble human TRβC domain.

29. The single-chain chimeric polypeptide of claim 1, wherein the soluble TRβC domain does not bind to an MHC protein with or without a loaded antigen and/or the soluble TRβC domain does not stimulate an immune response in a mammal.

30.-33. (canceled)

34. A composition comprising the single-chain chimeric polypeptide of claim 1.

35. (canceled)

36. A kit comprising at least one dose of the composition of claim 34.

37. A method of stimulating an immune cell, the method comprising:
contacting an immune cell with an effective amount of the single-chain chimeric polypeptide of claim 1.

38. A method of inducing or increasing proliferation of an immune cell, the method comprising:
contacting an immune cell with an effective amount of the single-chain chimeric polypeptide of claim 1.

39. A method of inducing differentiation of an immune cell into a memory or memory-like immune cell, the method comprising:
contacting an immune cell with an effective amount of the single-chain chimeric polypeptide of claim 1.

40.-44. (canceled)

45. A method of killing a cancer cell, an infected cell, or a senescent cell in a subject in need thereof, the method comprising administering to the subject a therapeutically effective amount of the single-chain chimeric polypeptide of claim 1.

46. A method of treating a subject in need thereof, the method comprising administering to the subject a therapeutically effective amount of the single-chain chimeric polypeptide of claim 1.

47.-50. (canceled)

51. Nucleic acid encoding the single-chain chimeric polypeptide of claim 1.

52. (canceled)

53. A cell comprising the nucleic acid of claim 51.

54. A method of producing a single-chain chimeric polypeptide, the method comprising:
culturing the cell of claim 53 in a culture medium under conditions sufficient to result in the production of the single-chain chimeric polypeptide; and
recovering the single-chain chimeric polypeptide from the cell and/or the culture medium.

55. (canceled)


SEQUENCE LISTING (137 sequences)

SEQ ID NO: 1 - protein, 130 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 2 - DNA, 390 bp, Homo sapiens
  source 1..390: mol_type = genomic DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 3 - protein, 130 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 5 - protein, 133 aa, Homo sapiens [sequence omitted]

SEQ ID NO: 6 - DNA, 399 bp, Homo sapiens
  source 1..399: mol_type = genomic DNA
caaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 7 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - DNA, 408 bp, synthetic construct
  source 1..408: mol_type = other DNA
atcccgccacacgtgcaaaagagcgtgaacaacgacatgatcgttacggacaacaatggggccgtgaagttccctcagttatgcaaattctgtgacgtgagattcagcacgtgcgacaatcagaagagctgcatgtccaattgctcaatcacaagcatctgcgagaaaccccaagaagtttgcgtggccgtgtggagaaagaatgacgagaacatcaccctggaaaccgtgtgccatgatcctaaactcccttaccatgactttatcttggaggatgctgctagcccaaagtgcatcatgaaagagaagaagaaaccgggggaaacttttttcatgtgttcatgcagctctgacgagtgtaatgataacattatcttcagcgaagagtataacactagcaatcctgat

SEQ ID NO: 9 - protein, 277 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - DNA, 831 bp, synthetic construct
  source 1..831: mol_type = other DNA
atcccgccacacgtgcaaaagagcgtgaacaacgacatgatcgttacggacaacaatggggccgtgaagttccctcagttatgcaaattctgtgacgtgagattcagcacgtgcgacaatcagaagagctgcatgtccaattgctcaatcacaagcatctgcgagaaaccccaagaagtttgcgtggccgtgtggagaaagaatgacgagaacatcaccctggaaaccgtgtgccatgatcctaaactcccttaccatgactttatcttggaggatgctgctagcccaaagtgcatcatgaaagagaagaagaaaccgggggaaacttttttcatgtgttcatgcagctctgacgagtgtaatgataacattatcttcagcgaagagtataacactagcaatcctgatggaggtggcggatctatccccccccatgtgcaaaaatcagtgaacaatgacatgatcgtgaccgataataacggggcggtaaagtttcctcaactgtgtaaattttgcgacgtcagattcagcacatgcgacaatcaaaagtcctgcatgagcaactgcagcatcacctctatctgtgagaaaccacaagaagtatgtgtggcggtatggcggaagaacgatgaaaatatcacgctggagaccgtttgccatgatcctaagctgccatatcacgatttcatcctggaggacgccgctagccctaagtgtatcatgaaggagaagaagaagcccggtgagacctttttcatgtgctcatgcagctccgacgagtgcaacgacaacatcatcttcagcgaggagtacaatacaagcaatcctgac

SEQ ID NO: 11 - DNA, 399 bp, synthetic construct
  source 1..399: mol_type = other DNA
cagggccaggacaggcacatgatccggatgaggcagctcatcgacatcgtcgaccagctgaagaactacgtgaacgacctggtgcccgagtttctgcctgcccccgaggacgtggagaccaactgcgagtggtccgccttctcctgctttcagaaggcccagctgaagtccgccaacaccggcaacaacgagcggatcatcaacgtgagcatcaagaagctgaagcggaagcctccctccacaaacgccggcaggaggcagaagcacaggctgacctgccccagctgtgactcctacgagaagaagccccccaaggagttcctggagaggttcaagtccctgctgcagaagatgatccatcagcacctgtcctccaggacccacggctccgaggactcc

SEQ ID NO: 12 - protein, 133 aa, Homo sapiens [sequence omitted]

SEQ ID NO: 13 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 14 - protein, 79 aa, Homo sapiens
EGAVLPRSAKELRCQCIKTYSKPFHPKFIKELRVIESGPHCANTEIIVKLSDGRELCLDPKENWVQRVVEKFLKRAENS

SEQ ID NO: 15 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 16 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 17 - DNA, 918 bp, Homo sapiens
  source 1..918: mol_type = genomic DNA
atttgggaactgaagaaggacgtctacgtggtcgaactggactggtatcccgatgctcccggcgaaatggtggtgctcacttgtgacacccccgaagaagacggcatcacttggaccctcgatcagagcagcgaggtgctgggctccggaaagaccctcacaatccaagttaaggagttcggagacgctggccaatacacatgccacaagggaggcgaggtgctcagccattccttattattattacacaagaaggaagacggaatctggtccaccgacattttaaaagatcagaaggagcccaagaataagacctttttaaggtgtgaggccaaaaactacagcggtcgtttcacttgttggtggctgaccaccatttccaccgatttaaccttctccgtgaaaagcagccggggaagctccgaccctcaaggtgtgacatgtggagccgctaccctcagcgctgagagggttcgtggcgataacaaggaatacgagtacagcgtggagtgccaagaagatagcgcttgtcccgctgccgaagaatctttacccattgaggtgatggtggacgccgtgcacaaactcaagtacgagaactacacctcctccttctttatccgggacatcattaagcccgatcctcctaagaatttacagctgaagcctctcaaaaatagccggcaagttgaggtctcttgggaatatcccgacacttggagcacaccccacagctacttctctttaaccttttgtgtgcaagttcaaggtaaaagcaagcgggagaagaaagaccgggtgtttaccgacaaaaccagcgccaccgtcatctgtcggaagaacgcctccatcagcgtgagggctcaagatcgttattactccagcagctggtccgagtgggccagcgtgccttgttcc

SEQ ID NO: 18 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - DNA, 591 bp, Homo sapiens
  source 1..591: mol_type = genomic DNA
cgtaacctccccgtggctacccccgatcccggaatgttcccttgtttacaccacagccagaatttactgagggccgtgagcaacatgctgcagaaagctaggcagactttagaattttacccttgcaccagcgaggagatcgaccatgaagatatcaccaaggacaagacatccaccgtggaggcttgtttacctctggagctgacaaagaacgagtcttgtctcaactctcgtgaaaccagcttcatcacaaatggctcttgtttagcttcccggaagacctcctttatgatggctttatgcctcagctccatctacgaggatttaaagatgtaccaagtggagttcaagaccatgaacgccaagctgctcatggaccctaaacggcagatctttttagaccagaacatgctggctgtgattgatgagctgatgcaagctttaaacttcaactccgagaccgtccctcagaagtcctccctcgaggagcccgatttttacaagacaaagatcaaactgtgcattttactccacgcctttaggatccgggccgtgaccattgaccgggtcatgagctatttaaacgccagc

SEQ ID NO: 20 - protein, 518 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - DNA, 1554 bp, Homo sapiens
  source 1..1554: mol_type = genomic DNA
atttgggaactgaagaaggacgtctacgtggtcgaactggactggtatcccgatgctcccggcgaaatggtggtgctcacttgtgacacccccgaagaagacggcatcacttggaccctcgatcagagcagcgaggtgctgggctccggaaagaccctcacaatccaagttaaggagttcggagacgctggccaatacacatgccacaagggaggcgaggtgctcagccattccttattattattacacaagaaggaagacggaatctggtccaccgacattttaaaagatcagaaggagcccaagaataagacctttttaaggtgtgaggccaaaaactacagcggtcgtttcacttgttggtggctgaccaccatttccaccgatttaaccttctccgtgaaaagcagccggggaagctccgaccctcaaggtgtgacatgtggagccgctaccctcagcgctgagagggttcgtggcgataacaaggaatacgagtacagcgtggagtgccaagaagatagcgcttgtcccgctgccgaagaatctttacccattgaggtgatggtggacgccgtgcacaaactcaagtacgagaactacacctcctccttctttatccgggacatcattaagcccgatcctcctaagaatttacagctgaagcctctcaaaaatagccggcaagttgaggtctcttgggaatatcccgacacttggagcacaccccacagctacttctctttaaccttttgtgtgcaagttcaaggtaaaagcaagcgggagaagaaagaccgggtgtttaccgacaaaaccagcgccaccgtcatctgtcggaagaacgcctccatcagcgtgagggctcaagatcgttattactccagcagctggtccgagtgggccagcgtgccttgttccggcggtggaggatccggaggaggtggctccggcggcggaggatctcgtaacctccccgtggctacccccgatcccggaatgttcccttgtttacaccacagccagaatttactgagggccgtgagcaacatgctgcagaaagctaggcagactttagaattttacccttgcaccagcgaggagatcgaccatgaagatatcaccaaggacaagacatccaccgtggaggcttgtttacctctggagctgacaaagaacgagtcttgtctcaactctcgtgaaaccagcttcatcacaaatggctcttgtttagcttcccggaagacctcctttatgatggctttatgcctcagctccatctacgaggatttaaagatgtaccaagtggagttcaagaccatgaacgccaagctgctcatggaccctaaacggcagatctttttagaccagaacatgctggctgtgattgatgagctgatgcaagctttaaacttcaactccgagaccgtccctcagaagtcctccctcgaggagcccgatttttacaagacaaagatcaaactgtgcattttactccacgcctttaggatccgggccgtgaccattgaccgggtcatgagctatttaaacgccagc

SEQ ID NO: 22 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 24 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 25 - DNA, 471 bp, Homo sapiens
  source 1..471: mol_type = genomic DNA
tacttcggcaaactggaatccaagctgagcgtgatccggaatttaaacgaccaagttctgtttatcgatcaaggtaaccggcctctgttcgaggacatgaccgactccgattgccgggacaatgccccccggaccatcttcattatctccatgtacaaggacagccagccccggggcatggctgtgacaattagcgtgaagtgtgagaaaatcagcactttatcttgtgagaacaagatcatctcctttaaggaaatgaacccccccgataacatcaaggacaccaagtccgatatcatcttcttccagcggtccgtgcccggtcacgataacaagatgcagttcgaatcctcctcctacgagggctactttttagcttgtgaaaaggagagggatttattcaagctgatcctcaagaaggaggacgagctgggcgatcgttccatcatgttcaccgtccaaaacgaggat

SEQ ID NO: 26 - protein, 352 aa, Homo sapiens
RDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 27 - protein, 248 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 28 - protein, 209 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 29 - protein, 360 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 30 - protein, 361 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDFPYVSAAMPCFVIIIILCVPCCKKKTSAAEGPELVSLQVLDQHPVGTGDHRDAAQLGFQPLMSATGSTGSTEGA

SEQ ID NO: 31 - protein, 190 aa, Homo sapiens
WVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 32 - protein, 191 aa, Homo sapiens
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMS

SEQ ID NO: 33 - protein, 188 aa, Homo sapiens
DAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPG

SEQ ID NO: 34 - protein, 233 aa, Homo sapiens
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 35 - protein, 193 aa, Homo sapiens
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSG

SEQ ID NO: 36 - protein, 193 aa, Homo sapiens
RRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSG

SEQ ID NO: 37 - DNA, 408 bp, synthetic construct
  source 1..408: mol_type = other DNA
atccccccccatgtgcaaaaatcagtgaacaatgacatgatcgtgaccgataataacggggcggtaaagtttcctcaactgtgtaaattttgcgacgtcagattcagcacatgcgacaatcaaaagtcctgcatgagcaactgcagcatcacctctatctgtgagaaaccacaagaagtatgtgtggcggtatggcggaagaacgatgaaaatatcacgctggagaccgtttgccatgatcctaagctgccatatcacgatttcatcctggaggacgccgctagccctaagtgtatcatgaaggagaagaagaagcccggtgagacctttttcatgtgctcatgcagctccgacgagtgcaacgacaacatcatcttcagcgaggagtacaatacaagcaatcctgac

SEQ ID NO: 38 - DNA, 408 bp, Homo sapiens
  source 1..408: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcacgatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgat

SEQ ID NO: 39 - protein, 287 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 40 - DNA, 861 bp, synthetic construct
  source 1..861: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcacgatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgcacaatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 41 - protein, 119 aa, synthetic construct [sequence omitted]

SEQ ID NO: 42 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 43 - DNA, 357 bp, synthetic construct
  source 1..357: mol_type = other DNA
caagttcagctccagcaaagcggcgccgaactcgctcggcccggcgcttccgtgaagatgtcttgtaaggcctccggctataccttcacccggtacacaatgcactgggtcaagcaacggcccggtcaaggtttagagtggattggctatatcaacccctcccggggctataccaactacaaccagaagttcaaggacaaagccaccctcaccaccgacaagtccagcagcaccgcttacatgcagctgagctctttaacatccgaggattccgccgtgtactactgcgctcggtactacgacgatcattactgcctcgattactggggccaaggtaccaccttaacagtctcctcc

SEQ ID NO: 44 - DNA, 321 bp, synthetic construct
  source 1..321: mol_type = other DNA
cagatcgtgctgacccagtcccccgctattatgagcgctagccccggtgaaaaggtgactatgacatgcagcgccagctcttccgtgagctacatgaactggtatcagcagaagtccggcaccagccctaaaaggtggatctacgacaccagcaagctggccagcggcgtccccgctcactttcggggctccggctccggaacaagctactctctgaccatcagcggcatggaagccgaggatgccgctacctattactgtcagcagtggagctccaaccccttcacctttggatccggcaccaagctcgagattaatcgt

SEQ ID NO: 45 - protein, 241 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 46 - DNA, 723 bp, synthetic construct
  source 1..723: mol_type = other DNA
cagatcgtgctgacccagtcccccgctattatgagcgctagccccggtgaaaaggtgactatgacatgcagcgccagctcttccgtgagctacatgaactggtatcagcagaagtccggcaccagccctaaaaggtggatctacgacaccagcaagctggccagcggcgtccccgctcactttcggggctccggctccggaacaagctactctctgaccatcagcggcatggaagccgaggatgccgctacctattactgtcagcagtggagctccaaccccttcacctttggatccggcaccaagctcgagattaatcgtggaggcggaggtagcggaggaggcggatccggcggtggaggtagccaagttcagctccagcaaagcggcgccgaactcgctcggcccggcgcttccgtgaagatgtcttgtaaggcctccggctataccttcacccggtacacaatgcactgggtcaagcaacggcccggtcaaggtttagagtggattggctatatcaacccctcccggggctataccaactacaaccagaagttcaaggacaaagccaccctcaccaccgacaagtccagcagcaccgcttacatgcagctgagctctttaacatccgaggattccgccgtgtactactgcgctcggtactacgacgatcattactgcctcgattactggggccaaggtaccaccttaacagtctcctcc

SEQ ID NO: 47 - protein, 107 aa, synthetic construct
DIEMTQSPAIMSASLGERVTMTCTASSSVSSSYFHWYQQKPGSSPKLCIYSTSNLASGVPPRFSGSGSTSYSLTISSMEAEDAATYFCHQYHRSPTFGGGTKLETKR

SEQ ID NO: 48 - protein, 114 aa, synthetic construct
VQLQQSGPELVKPGASVKMSCKASGYTFTSYVIQWVKQKPGQGLEWIGSINPYNDYTKYNEKFKGKATLTSDKSSITAYMEFSSLTSEDSALYYCARWGDGNYWGRGTTLTVSS

SEQ ID NO: 49 - DNA, 321 bp, synthetic construct
  source 1..321: mol_type = other DNA
gacatcgagatgacacagtcccccgctatcatgagcgcctctttaggagaacgtgtgaccatgacttgtacagcttcctccagcgtgagcagctcctatttccactggtaccagcagaaacccggctcctcccctaaactgtgtatctactccacaagcaatttagctagcggcgtgcctcctcgttttagcggctccggcagcacctcttactctttaaccattagctctatggaggccgaagatgccgccacatacttttgccatcagtaccaccggtcccctacctttggcggaggcacaaagctggagaccaagcgg

SEQ ID NO: 50 - DNA, 342 bp, synthetic construct
  source 1..342: mol_type = other DNA
gtgcagctgcagcagtccggacccgaactggtcaagcccggtgcctccgtgaaaatgtcttgtaaggcttctggctacacctttacctcctacgtcatccaatgggtgaagcagaagcccggtcaaggtctcgagtggatcggcagcatcaatccctacaacgattacaccaagtataacgaaaagtttaagggcaaggccactctgacaagcgacaagagctccattaccgcctacatggagttttcctctttaacttctgaggactccgctttatactattgcgctcgttggggcgatggcaattattggggccggggaactactttaacagtgagctcc

SEQ ID NO: 51 - protein, 236 aa, synthetic construct
VQLQQSGPELVKPGASVKMSCKASGYTFTSYVIQWVKQKPGQGLEWIGSINPYNDYTKYNEKFKGKATLTSDKSSITAYMEFSSLTSEDSALYYCARWGDGNYWGRGTTLTVSSGGGGSGGGGSGGGGSDIEMTQSPAIMSASLGERVTMTCTASSSVSSSYFHWYQQKPGSSPKLCIYSTSNLASGVPPRFSGSGSTSYSLTISSMEAEDAATYFCHQYHRSPTFGGGTKLETKR

SEQ ID NO: 52 - DNA, 708 bp, synthetic construct
  source 1..708: mol_type = other DNA
gtgcagctgcagcagtccggacccgaactggtcaagcccggtgcctccgtgaaaatgtcttgtaaggcttctggctacacctttacctcctacgtcatccaatgggtgaagcagaagcccggtcaaggtctcgagtggatcggcagcatcaatccctacaacgattacaccaagtataacgaaaagtttaagggcaaggccactctgacaagcgacaagagctccattaccgcctacatggagttttcctctttaacttctgaggactccgctttatactattgcgctcgttggggcgatggcaattattggggccggggaactactttaacagtgagctccggcggcggcggaagcggaggtggaggatctggcggtggaggcagcgacatcgagatgacacagtcccccgctatcatgagcgcctctttaggagaacgtgtgaccatgacttgtacagcttcctccagcgtgagcagctcctatttccactggtaccagcagaaacccggctcctcccctaaactgtgtatctactccacaagcaatttagctagcggcgtgcctcctcgttttagcggctccggcagcacctcttactctttaaccattagctctatggaggccgaagatgccgccacatacttttgccatcagtaccaccggtcccctacctttggcggaggcacaaagctggagaccaagcgg

SEQ ID NO: 53 - protein, 15 aa, synthetic construct
GGGGSGGGGSGGGGS

SEQ ID NO: 54 - DNA, 45 bp, synthetic construct
  source 1..45: mol_type = other DNA
ggcggtggaggatccggaggaggtggctccggcggcggaggatct

SEQ ID NO: 55 - protein, 8 aa, synthetic construct
GGGSGGGS

SEQ ID NO: 56 - protein, 4 aa, synthetic construct
GGGS

SEQ ID NO: 57 - protein, 18 aa, synthetic construct
MKWVTFISLLFLFSSAYS

SEQ ID NO: 58 - DNA, 54 bp, synthetic construct
  source 1..54: mol_type = other DNA
atgaaatgggtgacctttatttctttactgttcctctttagcagcgcctactcc

SEQ ID NO: 59 - DNA, 54 bp, synthetic construct
  source 1..54: mol_type = other DNA
atgaagtgggtcacatttatctctttactgttcctcttctccagcgcctacagc

SEQ ID NO: 60 - protein, 16 aa, synthetic construct
MKCLLYLAFLFLGVNC

SEQ ID NO: 61 - protein, 58 aa, synthetic construct
MGQIVTMFEALPHIIDEVINIVIIVLIIITSIKAVYNFATCGILALVSFLFLAGRSCG

SEQ ID NO: 62 - protein, 97 aa, synthetic construct
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 63 - protein, 30 aa, synthetic construct
MDSKGSSQKGSRLLLLLVVSNLLLCQGVVS

SEQ ID NO: 64 - DNA, 456 bp, synthetic construct
  source 1..456: mol_type = other DNA
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 65 - protein, 15 aa, synthetic construct
GLNDIFEAQKIEWHE

SEQ ID NO: 66 - protein, 26 aa, synthetic construct
KRRWKKNFIAVSAANRFKKISSSGAL

SEQ ID NO: 67 - protein, 6 aa, synthetic construct
EEEEEE

SEQ ID NO: 68 - protein, 13 aa, synthetic construct
GAPVPYPDPLEPR

SEQ ID NO: 69 - protein, 8 aa, synthetic construct
DYKDDDDK

SEQ ID NO: 70 - protein, 9 aa, synthetic construct
YPYDVPDYA

SEQ ID NO: 71 - protein, 5 aa, synthetic construct
HHHHH

SEQ ID NO: 72 - protein, 6 aa, synthetic construct
HHHHHH

SEQ ID NO: 73 - protein, 7 aa, synthetic construct
HHHHHHH

SEQ ID NO: 74 - protein, 8 aa, synthetic construct
HHHHHHHH

SEQ ID NO: 75 - protein, 9 aa, synthetic construct
HHHHHHHHH

SEQ ID NO: 76 - protein, 10 aa, synthetic construct
HHHHHHHHHH

SEQ ID NO: 77 - protein, 10 aa, synthetic construct
EQKLISEEDL

SEQ ID NO: 78 - protein, 18 aa, synthetic construct
TKENPRSNQEESYDDNES

SEQ ID NO: 79 - protein, 15 aa, synthetic construct
KETAAAKFERQHMDS

SEQ ID NO: 80 - protein, 38 aa, synthetic construct
MDEKTTGWRGGHVVEGLAGELEQLRARLEHHPQGQREP

SEQ ID NO: 81 - protein, 13 aa, synthetic construct
SLAELLNAGLGGS

SEQ ID NO: 82 - protein, 8 aa, synthetic construct
TQDPSRVG

SEQ ID NO: 83 - protein, 12 aa, synthetic construct
PDRVRAVSHWSS

SEQ ID NO: 84 - protein, 8 aa, synthetic construct
WSHPQFEK

SEQ ID NO: 85 - protein, 6 aa, synthetic construct
CCPGCC

SEQ ID NO: 86 - protein, 10 aa, synthetic construct
EVHTNQDPLD

SEQ ID NO: 87 - protein, 14 aa, synthetic construct
GKPIPNPLLGLDST

SEQ ID NO: 88 - protein, 11 aa, synthetic construct
YTDIEMNRLGK

SEQ ID NO: 89 - protein, 8 aa, synthetic construct
DLYDDDDK

SEQ ID NO: 90 - protein, 120 aa, synthetic construct
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 91 - protein, 153 aa, synthetic construct [sequence omitted]

SEQ ID NO: 92 - DNA, 831 bp, synthetic construct
  source 1..831: mol_type = other DNA
attcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgac

SEQ ID NO: 93 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 94 - DNA, 732 bp, synthetic construct
  source 1..732: mol_type = other DNA
gatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctcc

SEQ ID NO: 95 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 96 - DNA, 729 bp, synthetic construct
  source 1..729: mol_type = other DNA
gacatcaagctgcagcagagcggcgccgagctggctagacctggggctagcgtcaagatgagctgcaagacaagcggctacactttcacaagatacaccatgcactgggtgaagcagagacctggccaaggcctggaatggatcggttatatcaaccctagcagaggctacaccaactacaatcagaagttcaaggacaaagccaccctgacaactgataagagcagcagcaccgcctatatgcaactgtcaagcctcacctcagaggacagcgccgtgtactactgcgctagatactacgacgaccactactgcctggactattggggccaaggcactactttgactgttagctcggtcgagggaggctccggcgggagtggtggttctggcggcagcggcggagttgacgacattcagctgacacaaagtcctgccataatgagcgctagccctggcgagaaggtgaccatgacctgcagagctagcagcagcgtgagctacatgaactggtatcagcagaagagcggcacaagccctaaaagatggatctacgacacaagcaaggtggctagcggcgtgccttaccggtttagcggctctggtagcggcacaagctatagcctcacgattagctcaatggaggccgaggacgccgccacgtattattgccaacagtggagcagcaaccctctgaccttcggcgccgggaccaagctggaacttaag

SEQ ID NO: 97 - protein, 238 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 98 - protein, 229 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 99 - protein, 473 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 100 - DNA, 1419 bp, synthetic construct
  source 1..1419: mol_type = other DNA
gatattcagatgacacagtcacctgcctcactgtcagcgtctgtgggggaccgcgtgacaattacgtgcctggctagccaaaccatagatacctggctggcgtggtacttgcagaaacctggaaagtcgccacagctgctgatctatgcggcaactaacctggcggacggcgtaccctcgcgtttttccgggtctggcagcggtaccgatttctccttcactattagcagccttcagccggaggactttgctacatattattgtcagcaagtgtactcatcccctttcaccttcggccaaggcaccaaactagaaataaaaggcgggggcggttcacaaattcagctggtgcaaagcggcggcgaagtgaagaagcctggagctagcgtgcgtgtgagctgtaaggcatctggctactccttcactgattacaacgtgtattgggttagacagtcccctggtaaagggcttgagtggattggctatatagacccttataacggcattaccatatacgatcaaaacttcaaaggtaaggccaccctgaccgttgacaagtcaacaagcaccgcctacatggagctgagcagcctaagaagcgaggacaccgctgtctatttctgtgctagagacgtaaccacagccctggatttctggggccaaggcacaacggtgacagtcagctcgggtggcggcggttcgggtgggggaggaagcgggggggggggatccgacattcaaatgacgcaaagccctgctagcctttccgctagcgtgggagaccgcgttacgatcacatgtctggcctctcagaccatcgacacttggctggcctggtacctacaaaaacctggcaaaagcccacaactactgatctatgctgctacgaatctggccgacggcgtaccgtcccgcttcagcggctccggttctgggacagacttctctttcaccatttcatctctgcagcctgaagatttcgccacatattattgtcaacaagtgtatagcagcccgtttacctttgggcaaggcacgaagttagaaatcaagggcggaggcggatcccaaattcagctcgtgcagagtggcggggaagtcaagaaacctggcgctagcgtgagagtgtcttgtaaggcaagtggctacagctttaccgactataatgtgtattgggtgagacaatcacccggcaaggggctggagtggatcggctacatcgacccgtataacggcattacgatctacgaccaaaactttaaaggcaaggccaccctaacggtcgacaagtcgacctccaccgcttatatggagcttagctcattgagaagcgaggataccgcggtgtacttctgcgctagagacgtgacaaccgccctggacttctgggggcaaggcacaaccgtcaccgtgtcgtcc

SEQ ID NO: 101 - protein, 117 aa, synthetic construct
QIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 102 - protein, 107 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIK

SEQ ID NO: 103 - protein, 239 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 104 - DNA, 717 bp, synthetic construct
  source 1..717: mol_type = other DNA
gacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttcc

SEQ ID NO: 105 - protein, 493 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGS

SEQ ID NO: 106 - DNA, 1479 bp, synthetic construct
  source 1..1479: mol_type = other DNA
gacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttccggcggtgggggctcggatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctccggcggtgggggctcg

SEQ ID NO: 107 - protein, 236 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 108 - protein, 487 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 109 - DNA, 1461 bp, synthetic construct
  source 1..1461: mol_type = other DNA
caagtgcagctggtgcagagcggcgtggaggtgaagaagccgggcgctagcgtgaaggtgagctgcaaggcctccggatataccttcacaaactactacatgtattgggttagacaagcccctggtcaaggcctggagtggatgggcggtataaacccttccaatggaggtactaatttcaacgaaaaattcaagaaccgtgtgaccctgaccaccgacagtagcaccaccactgcatacatggagctgaagagtctgcagttcgacgataccgcggtctactactgtgcccgacgagactatagatttgacatgggcttcgactactggggacaaggcactacggtgaccgtgagcagcggtggcggcggctccgaaattgtgctgacacagtcgcctgcaaccctgtccttgagccctggcgagcgggcaactctgagctgccgagctagcaaaggcgtgagtacaagcggctatagctacctacattggtatcagcaaaagcctggccaagccccgcgactgctgatctacctggctagctacctggagagcggcgttcctgctagattcagcggatctggctctggtactgacttcaccttaactatcagtagccttgaacccgaggacttcgctgtgtactattgtcagcacagcagagacctccctctgacctttggaggtggtaccaaggtcgaaatcaaaggaggcggagggtcgggcggtggtgggtctggaggtggaggaagccaagttcaactcgtgcagagcggtgtggaagttaaaaagcccggcgctagcgtaaaggtgagctgtaaggctagcggctacaccttcaccaattactacatgtactgggttaggcaagcccctggacaaggcttagaatggatgggtgggataaacccttcgaatggtggaaccaactttaacgagaagttcaaaaacagagtgacgttgaccaccgactctagcaccactaccgcgtacatggaactgaagagcctgcagttcgatgatacagcggtgtattattgcgcccgaagagactaccgattcgacatggggtttgactactggggccaaggcaccaccgtcaccgtcagcagcggcggaggcggtagtgagattgttctgacccaaagccctgccaccctctcgttaagcccgggggagagagcaaccctgagctgtagagctagcaagggcgtgagcacctcgggctacagttacctgcactggtaccaacagaagcctggccaagcccctcgattattgatctacttagctagctacttagaaagcggcgtgcccgctagattcagcggcagcgggagcggcaccgactttacactgacgataagtagcttagagcctgaggactttgcagtgtattactgccaacattcccgggacctgccgcttaccttcgggggcggcaccaaagtggagattaaa

SEQ ID NO: 110 - protein, 491 aa, synthetic construct
QVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELK

SEQ ID NO: 111 - protein, 246 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 112 - protein, 248 aa, synthetic construct
RTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 113 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - protein, 163 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESV

SEQ ID NO: 123 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 124 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 240 aa, synthetic construct
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGHGGGGSGGGGSGGGGSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 126 - DNA, 720 bp, synthetic construct
  source 1..720: mol_type = other DNA
agcgagctgacccaagaccctgccgtgagcgtggccctgggacagaccgtgagaatcacctgccaaggcgacagcctgagaagctactacgctagctggtatcagcagaagcctggccaagcccctgtgctggtgatctacggcaagaacaacagacctagcggcatccctgacagattcagcggcagcagcagcggcaacaccgctagcctgaccatcaccggcgcccaagccgaggacgaggccgactactactgcaacagcagagacagcagcggcaaccacgtggtcttcggcggaggaaccaaactcaccgtcggccacggcggaggcggaagcggcggcggaggctccggcggaggcggcagcgaagtgcagctggtggagagcggaggaggcgtcgtcagacctggcggcagcctgagactgagctgcgccgctagcggcttcaccttcgacgactacggcatgagctgggtgagacaagcccctggcaagggcctggagtgggtgagcggcatcaactggaacggcggcagcaccggctacgccgacagcgtgaagggcagattcaccatcagcagagacaacgccaagaacagcctgtacctgcagatgaacagcctgagagccgaggacaccgccgtgtactactgcgctagaggcagaagcctgctgttcgactactggggccaaggcaccctggtgaccgtgagcaga

SEQ ID NO: 127 - protein, 255 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 128 - DNA, 765 bp, synthetic construct
  source 1..765: mol_type = other DNA
tcttatgtaagaccgctctccgtcgcattgggggagaccgcgcgcattagttgtggacgacaagcacttggatctcgagcagttcagtggtaccagcaccgacccggacaagctcctattctgctcatttataataaccaagacagaccatcaggcatccccgagcgattctctgggaccccggatatcaatttcggaaccagggcgacacttactatttcaggtgttgaggccggggacgaggcagactattactgccacatgtgggacagccgatctggattttcttggtcatttggaggcgcaacacgcttgaccgttctcggagggggtggctcaggaggcggcggatcaggagggggaggttcacaagtgcaacttcaggaatccggacctggactcgtgaagccttcagaaaccctttcagtaacgtgcagcgtgagcggagatagcatgaacaactattactggacttggataagacaatctccgggcaaggggctcgagtggatagggtatatctcagatagagaatccgccacttataatcctagccttaacagccgggtagtcataagcagagacacaagcaaaaatcaactcagtttgaaattgaattcagtaacacctgccgacaccgcagtgtactattgcgcgacagcccggcgagggcagaggatttatggcgtcgttagctttggagaatttttctactattactccatggatgtgtggggcaaggggaccacggttactgtgtcttca

SEQ ID NO: 129 - protein, 17 aa, synthetic construct
MGVKVLFALICIAVAEA

SEQ ID NO: 130 - protein, 17 aa, synthetic construct
MERTLVCLVVIFLGTVA

SEQ ID NO: 131 - protein, 19 aa, synthetic construct
MGWSCIILFLVATATGVHS

SEQ ID NO: 132 - protein, 129 aa, synthetic construct
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 133 - DNA, 387 bp, synthetic construct
  source 1..387: mol_type = other DNA
cataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcc

SEQ ID NO: 134 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 450 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 213 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC